SEQUENCE LISTING (117 sequences)

SEQ ID NO: 1 - DNA, 31 bp, artificial sequence
  Primer Tef-F
gaattcactagtggggttcaaatgcaaacaa

SEQ ID NO: 2 - DNA, 25 bp, artificial sequence
  Primer Tef-R
ggatcctggtgcgaactttgtagtt

SEQ ID NO: 3 - DNA, 24 bp, artificial sequence
  Primer nia-F
ctcgagattatccaagggaatgac

SEQ ID NO: 4 - DNA, 25 bp, artificial sequence
  Primer nia-R
tctagaaagtattttcggtacgatt

SEQ ID NO: 5 - DNA, 30 bp, artificial sequence
  Primer hph-F
ggatcctacacctcagcaatgtcgcctgaa

SEQ ID NO: 6 - DNA, 33 bp, artificial sequence
  Primer hph-R
ctcgagctattcctttgccctcggacgagtgct

SEQ ID NO: 7 - DNA, 2221 bp, artificial sequence
  Hygromycin B resistance gene (hph) expression parts in plasmid pHUda966 .
gaattcactagtggggttcaaatgcaaacaagtacaacacgcagcaaacgaagcagcccaccactgcgttgatgcccagtttgactgtccgaaatccaccggaaaggtggaaacatactatgtaacaatcagagggaagaaaaaatttttatcgacgaggcaggatagtgactgatggtggggtcatggtcgggtctccgagcgaaagagaaccaaggaaacaagatcaacgaggttggtgtacccaaaaggccgcagcaacaagagtcatcgcccaaaagtcaacagtctggaagagactccgccgtgcagattctgcgtcggtcccgcacatgcgtggtgggggcattacccctccatgtccaatgataagggcggcggtcgagggcttaagcccgcccactaattcgccttctcgcttgcccctccatataaggattccccctccttcccctcccacaacttttttccttctttctctcttcgtccgcatcagtacgtatatctttcccccatacctcctttcctactcttcttccattcattcaactcttctccttactgacatctgttttgctcagtacctctacgcgatcagccgtagtatctgagcaagcttctctacagaatctttctagtatcttacaaagaactacaaagttcgcaccaggatcctacacctcagcaatgtcgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagctcgagattatccaagggaatgacttaatgagtatgtaagacatgggtcataacggcgttcgaaacatatacagggttatgtttgggaatagcacacgaataataacgttaataggtaccaaagtccttgatacattagcacggtagaaaaagaataatacaacgagctgggaatattctttaatataaaactccaagaagagctggtgcggtggagcttgttttcgactctcagtaatatttcctcatatccaagcgcgctaggaggtggtcgaatacacatgtaggcgcttctctggatgcaaaagtcgtgccggacctgccgaaagactttgaagatgcgttcacgccatctaagttgcgtagataattcacaaaaagggatgtttgtttccggaatgtagcaaagagctgataggcaatagcctcactttcgtggcgcacgccgctcgttccatccatcctcgacaatggagcaaatgtcaaaatcgtaccgaaaatactttctaga

SEQ ID NO: 8 - protein, 340 aa, artificial sequence
  Synthetic Construct
MSPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 9 - DNA, 49 bp, artificial sequence
  FRT-F site
ttgaagttcctattccgagttcctattctctagaaagtataggaacttc

SEQ ID NO: 10 - DNA, 50 bp, artificial sequence
  FRT-F3 site
ttgaagttcctattccgagttcctattcttcaaatagtataggaacttca

SEQ ID NO: 11 - DNA, 77 bp, artificial sequence
  Primer 3NA1-F
actagtttgaagttcctattccgagttcctattcttcaaatagtataggaacttcaactagagtatatgatggtact

SEQ ID NO: 12 - DNA, 32 bp, artificial sequence
  Primer 3NA1-R
gaattcgcattctcctagttactgatgacttt

SEQ ID NO: 13 - DNA, 28 bp, artificial sequence
  Primer 5NA1-F
gcggccgcgtttaaacctatctgttccc

SEQ ID NO: 14 - DNA, 82 bp, artificial sequence
  Primer 5NA1-R
actagtgctagcgaagttcctatactttctagagaataggaactcggaataggaacttcaagatgaattcgcggcctacatg

SEQ ID NO: 15 - DNA, 5890 bp, artificial sequence
  NA1-encoding part and flanking regions of pHUda981.
acgaggtcctaaactatctgttccctccccccccttttatcttcttgtagtccggccttctagagaaaccatctgcgctgttctgctcgccagggaggtatgaccacgtcagcctaaagcgtccagcgaataaaatccatctgttcatccttcgattcgtcatgctttcctttagttcgtaagcaaggttcttgtgatcagtctgtacacgtatgcccggagatccttccaaaaggggaaaccatttctctagtgcgtagatcactgccaaaagttctcgttcggtgacggtgtagttgcgttcaggtggggtcaggcgccgggaaataatcgcgcaagttaggccgccttgcataagttgggcaccgattgcaaatgatgacgcgtccgttctcaaagtgcactttctggtggggtcgaagtaggctgtgtcgagcatccgttgctccaatcgtttcacattctcaaatgccaagtcttggcgccaagtccattttccgtcttgctttgtggcgtcgtaaagcggggtcgcgtggtgggccaacatcggtatgtaatcacggaaaaagttaaccacgcccaaaaacttccgaagttctgtcttattcctcggtttcggccagttgcgtatcgttccatcggagataaccgggctgcatctgttgtaactgtatcgatggccgcaataaacaacttctcgtacttttcgctggcatttcctttctttcaaagccaagccgttctgcctcaggcgtgtctcaatgccttgacaaatcctgtcatgttcctgttcgttgtcggagaaaaccaaaatatcgtccaagtgtatcgtaacattgttaccaagaaattcccacagtacattttcgatgtaaatctgccactctgctggggccgtgccgattccgaatggtaataccgtgtactggtatgttcccatgtgacatctaaacgtcgtcaaaggtctgtcttctttccgtattgtcatcttgtaatacgcttcctcaatgtcgtatttcgaaaagaaacgggctttctttatccaatccctgtggtaagattgatcgtcaggagattatctgcaggaaacatcatggtggggtaaccaaggttgtgtctgtataatatatacatgtaagatacatgagcttcggtgatataatacagaagtaccatacagtaccgcgttatgaaaacacattaatccggatcctttcctataatagactagcgtgcttggcattagggttcgaaaaacaatcgaagagtataaggggatgacagcagtaacgactccaactgtacgcctccgggtagtagaccgagcagccgagccagctcagcgcctaaaacgccttatacaattaagcagttaaagaagttagaatctacgcttaaaaagctacttaaaaatcgatctcgcagtcccgattcgcctatcaaaaccagtttaaatcaactgattaaaggtgccgaacgagctataaatgatataacaatattaaagcattaattagagcaatatcaggccgcgcacgaaaggcaacttaaaaagcgaaagcgctctactaaacagattacttttgaaaaaggcacatcagtatttaaagcccgaatccttattaagcgccgaaatcaggcagataaagccatacaggcagatagacctctacctattaaatcggcttctaggcgcgctccatctaaatgttctggctgtggtgtacaggggcataaaattacgcactacccgaatcgatagaactactcatttttatatagaagtcagaattcatggtgttttgatcattttaaatttttatatggcgggtggtgggcaactcgcttgcgcgggcaactcgcttaccgattacgttagggctgatatttacgtaaaaatcgtcaagggatgcaagaccaaagtagtaaaaccccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttaaagggattaatttccactcaaccacaaatcacagtcgtccccggtattgtcctgcagaatgcaatttaaactcttctgcgaatcgcttggattccccgcccctggccgtagagcttaaagtatgtcccttgtcgatgcgatgtatcacaacatataaatactagcaagggatgccatgcttggaggatagcaaccgacaacatcacatcaagctctcccttctctgaacaataaaccccacagaaggcatttatgatggtcgcgtggtggtctctatttctgtacggccttcaggtcgcggcacctgctttggctgcaacgcctgcggactggcgatcgcaatccatttatttccttctcacggatcgatttgcaaggacggatgggtcgacgactgcgacttgtaatactgcggatcaggtgtgttgttacctactagctttcagaaagaggaatgtaaactgacttgatatagaaatactgtggtggaacatggcagggcatcatcgacaaggtaaattgcccctttatcaaaaaaaaagaaggaaaagcagaagaaaaataaaataaaaagaactctagtcctaaccatcacatagttggactatatccagggaatgggcttcacagccatctggatcacccccgttacagcccagctgccccagaccaccgcatatggagatgcctaccatggctactggcagcaggatatgtaagtcgatttctttaaatatctacctgtcatcttttacatcaatatgaactaacttgatggttttagatactctctgaacgaaaactacggcactgcagatgacttgaaggcgctctcttcggcccttcatgagagggggatgtatcttatggtcgatgtggttgctaaccatatggttcgtggtcctttgcaactgacttcgcggatatggttcatttcagtactgacaatgagtaatatcagggctatgatggagcgggtagctcagtcgattacagtgtgtttaaaccgttcagttcccaagactacttccacccgttctgtttcattcaaaactatgaagatcagactcaggttgaggattgctggctaggagataacactgtctccttgcctgatctcgataccaccaaggatgtggtcaagaatgaatggtacgactgggtgggatcattggtatcgaactactccagtaagatatttctccctcattctacaacttggctgatcgatgatacttacgaaatcagttgacggcctccgtatcgacacagtaaaacacgtccagaaggacttctggcccgggtacaacaaagccgcaggcgtgtactgtatcggcgaggtgctcgacggtgatccggcctacacttgtccctaccagaacgtcatggacggcgtactgaactatcccatgtatggttcctccaaccatgagccttcttgcaagtctcatctcctaacgaaacggctaaaaccagttactatccactcctcaacgccttcaagtcaacctccggcagcatggacgacctctacaacatgatcaacaccgtcaaatccgactgtccagactcaacactcctgggcacattcgtcgagaaccacgacaacccacggttcgcttcgtaagtcttcccttttattttccgttcccaatttccacacagaaccccacctaacaagagcaaagttacaccaacgacatagccctcgccaagaacgtcgcagcattcatcatcctcaacgacggaatccccatcatctacgccggccaagaacagcactacgccggcggaaacgaccccgcgaaccgcgaagcaacctggctctcgggctacccgaccgacagcgagctgtacaagttaattgcctccgcgaacgcaatccggaactatgccattagcaaagatacaggattcgtgacctacaaggtaagcacaacctctaagcataccctaatggcctatcttcagagtatctgacacaagagactaatcactggcaatacagaactggcccatctacaaagacgacacaacgatcgccatgcgcaagggcacagatgggtcgcagatcgtgactatcttgtccaacaagggtgcttcgggtgattcgtataccctctccttgagtggtgcgggttacacagccggccagcaattgacggaggtcattggctgcacgaccgtgacggttggttcggatggaaatgtgcctgttcctatggcaggtgggctacctagggtattgtatccgactgagaagttggcaggtagcaagatctgtagtagctcgtgaagggtggagagtatatgatggtactgctattcaatctggcattggacagtgagtttgagtttgatgtacagttggagtcgttactgctgtcatccccttatactcttcgattgtttttcgaaccctaatgccaagcacgctagtctattataggaaaggatccggattaatgtgttttcataacgcggtactgtatggtacttctgtattatatcaccgaagctcatgtatcttacatgtatatattatacagacacaaccttggttacagttggagtcattactgctgtcaccccccccaatactctttgatcgtatttcgaaccctaatgccaagtgcgctagtctacatatggaaggtaaccgtaacattaatattccggaaattttgatcgtactgtattgaacagtaaggttatagaaatagtgtattgagtttgtatcagtaatctacggtagctggaagcttctacactgcaaacgcgtcaaacatgacaaagcatgtgccttgcatctcccgcaaactgttaacattccttttgtttgtactgagccacggttcgatcctttttgaccttaccaggttaaccaagacgggtagggctacaatactgtacctgtcttagttcattgtccatgaacctgatcattttactggttttgttagctgcacctcttccctcacggacactcttgctgggacacccatatggtgtaggctaacatatgatgatcccaacactaggcttctcagtggcatctactgccttgaggggatgacgtttagtccttactacgatgacgatgcctctcagcttcagccacctgatccgtggatacaaactccatcgtatgcccccacccctggtgacttcggtagagatcgtacgccatccgtattttgcccatcacctgaacatgtacttgatgaaccttatactcccttgcatcaatcccagcccaatcatctgggtttcttccaggagcccgaagaaaggactacgaggcaacatagaggaaagccttgtattcattatactattgagtggaaggtaactctgaacaaccgaactgtgtcaaaggacactgaacaggacttggctgtagcacccagttcacactgggcgaagataacacaggatgctgaaaatgttatgcgtcgaaaaatacgtcacaaccaacgtgtgagatcagatgatactacagtcagagtatctgtaaacgaacgtggacaatctgatctgaacaaacgttttgacggcactaatattgattggaaacctatagagaaacagctcttaatgtggggaaatctgtttcatattggcaagaagctcaaactttttatatccataaactatatagaggacagtggccctcctctttcacggaatacagataagagaggaaagtcatcagtaactaggagaatgcttacaga

SEQ ID NO: 16 - protein, 499 aa, artificial sequence
  Synthetic Construct
MMVAWWSLFLYGLQVAAPALAATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 17 - DNA, 20 bp, artificial sequence
  Forward primer
aatccggatcctttcctata

SEQ ID NO: 18 - DNA, 20 bp, artificial sequence
  Reverse primer
gatggagcgcgcctagaagc

SEQ ID NO: 19 - DNA, 24 bp, artificial sequence
  Primer amdS-F
ggatccaccatgcctcaatcctgg

SEQ ID NO: 20 - DNA, 30 bp, artificial sequence
  Primer amdS-R
ctcgagctatggagtcaccacatttcccag

SEQ ID NO: 21 - DNA, 3091 bp, artificial sequence
  The Aspergillus nidulans acetoamidase gene (amdS) expression parts in pHUda976.
gaattcactagtggggttcaaatgcaaacaagtacaacacgcagcaaacgaagcagcccaccactgcgttgatgcccagtttgactgtccgaaatccaccggaaaggtggaaacatactatgtaacaatcagagggaagaaaaaatttttatcgacgaggcaggatagtgactgatggtggggtcatggtcgggtctccgagcgaaagagaaccaaggaaacaagatcaacgaggttggtgtacccaaaaggccgcagcaacaagagtcatcgcccaaaagtcaacagtctggaagagactccgccgtgcagattctgcgtcggtcccgcacatgcgtggtgggggcattacccctccatgtccaatgataagggcggcggtcgagggcttaagcccgcccactaattcgccttctcgcttgcccctccatataaggattccccctccttcccctcccacaacttttttccttctttctctcttcgtccgcatcagtacgtatatctttcccccatacctcctttcctactcttcttccattcattcaactcttctccttactgacatctgttttgctcagtacctctacgcgatcagccgtagtatctgagcaagcttctctacagaatctttctagtatcttacaaagaactacaaagttcgcaccaggatccacagaatgcctcaatcctgggaagaactggccgctgataagcgcgcccgcctcgcaaaaaccatccctgatgaatggaaagtccagacgctgcctgcggaagacagcgttattgatttcccaaagaaatcgggtatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctcgagattatccaagggaatgacttaatgagtatgtaagacatgggtcataacggcgttcgaaacatatacagggttatgtttgggaatagcacacgaataataacgttaataggtaccaaagtccttgatacattagcacggtagaaaaagaataatacaacgagctgggaatattctttaatataaaactccaagaagagctggtgcggtggagcttgttttcgactctcagtaatatttcctcatatccaagcgcgctaggaggtggtcgaatacacatgtaggcgcttctctggatgcaaaagtcgtgccggacctgccgaaagactttgaagatgcgttcacgccatctaagttgcgtagataattcacaaaaagggatgtttgtttccggaatgtagcaaagagctgataggcaatagcctcactttcgtggcgcacgccgctcgttccatccatcctcgacaatggagcaaatgtcaaaatcgtaccgaaaatactttctaga

SEQ ID NO: 22 - protein, 548 aa, artificial sequence
  Synthetic Construct
MPQSWEELAADKRARLAKTIPDEWKVQTLPAEDSVIDFPKKSGILSEAELKITEASAADLVSKLAAGELTSVEVTLAFCKRAAIAQQLTNCAHEFFPDAALAQARELDEYYAKHKRPVGPLHGLPISLKDQLRVKGYETSMGYISWLNKYDEGDSVLTTMLRKAGAVFYVKTSVPQTLMVCETVNNIIGRTVNPRNKNWSCGGSSGGEGAIVGIRGGVIGVGTDIGGSIRVPAAFNFLYGLRPSHGRLPYAKMANSMEGQETVHSVVGPITHSVEDLRLFTKSVLGQEPWKYDSKVIPMPWRQSESDIIASKIKNGGLNIGYYNFDGNVLPHPPILRGVETTVAALAKAGHTVTPWTPYKHDFGHDLISHIYAADGSADVMRDISASGEPAIPNIKDLLNPNIKAVNMNELWDTHLQKWNYQMEYLEKWREAEEKAGKELDAIIAPITPTAAVRHDQFRYYGYASVINLLDFTSVVVPVTFADKNIDKKNESFKAVSELDALVQEEYDPEAYHGAPVAVQVIGRRLSEERTLAIAEEVGKLLGNVVTP

SEQ ID NO: 23 - DNA, 85 bp, artificial sequence
  Primer 3SP-F
actagtttgaagttcctattccgagttcctattcttcaaatagtataggaacttcaactagagaatgcaatcataacagaaagta

SEQ ID NO: 24 - DNA, 32 bp, artificial sequence
  Primer 3SP-R
gaattcttaattaaatcacggcaagggtttac

SEQ ID NO: 25 - DNA, 26 bp, artificial sequence
  Primer 5SP-F
ccgcggcaacaggcagaatatcttcc

SEQ ID NO: 26 - DNA, 77 bp, artificial sequence
  Primer 5SP-R
actagtgaagttcctatactttctagagaataggaactcggaataggaacttcaaacgggatcttggacgcattcca

SEQ ID NO: 27 - DNA, 6950 bp, Artificial sequence
  Acid stable amylase from Aspergillus niger with flanking sequence from  plasmid pHUDa1019.
cggcaacaggcagaatatcttccgaattcaatcgactgcgcgatgcaagttggctagcaacggcgtacaccttgggattatgcgctgctcaaccgatggtcagctatcaaacaaaatttgggaagatcgggctatactgacggtgacattatagtacggcaagctgagtgacatctacggtcgcaagccactgcttctttgggcatatgttttctttggcgtgggatgcattatcaggtagatactccctttttcttatacgctggtttgctggttcgtgctgacagctgtttccctagcggtattggtcgagacatggcgactgtcatattggggcgtgcaatcagcggaattgggggtgctggaacaatggcgatgggctctatcattatcacaggtaggctagcagcttatcaggttgaaagaactgtcactgaacataggcagatattgttcctcgtcgagatgttgcccattggcgggcgtacatcaatatcgcgatgactctgggtcgtagcgcaggaggcccaatcggcggatggctaaccgatacaatcggatggagatggtatgctttgcgcctttgtgaccgcttctctcactaaattgtggccaaggtcgtttattatccaaggccccttagccgctgtggcagctctgttggtgatatggaagctcaaactcgccaatccagtcactgagaagagcatccgccgtgtcgactttctcggaacattcctcctggccgtcggtattgttacaatcaccgttatcatggaccaagcagggcagtccttcgcatgggcatcattgtcaacagcaatccttgcaactctcagtctatcagcattcgtcgccttcgtccttgttgaactctacgtagcccctgaaccgattttcgaacttcgcatgttgcggaagccgaatgtgacgcccagttacctgatcggatcgctgcagatcaccgcccaagttggaatgatgttctccgtgccgttatattttcaggtgacatcgaaagcctctgccaccgtagctggagggcatctggttcctgcagtgatcggaaacacgcttggcggcttaatcgcgggagcctttatccgtcgcaccggccaattcaaggtcctcttgatccttgccggtctcgttgcgtccgtcgcctatctactcctcatccttcgctggaacggtcatactggattctgggagtccttgtacattattcccggtggtatgggtactggtttctgctctgcagctgcttttgtcagtatgacggcgtttttgatgccgcaggaagtggccatggcaacaggaggttacttcctattattcagcttcgccatgacggccggtgtcactgtcactaacagtctgctggggacggttttcaagcgccagatggaacagcacctgacgggtccaggagccaagaaggttggtatccccgcaccttttctgcgtcacttactaacgagtatatgaagatcatcgagcgcgcgctgtccgacaccagctatatcaacggtttgcagggtcatgtccgggatgtagtggtaaaaggatatgtgactggtctccgctacacttactgtaagtcgtttgaatcatgcatccaccgtccaccttattaacttggtgccagtattttccctcattctttcgctccttggatcggtcctcgcttggactgtacgaaaacaccaactatgaggaaccagcacggcagctgatagtatccgaaagctgcaaattgcttcatcgaggctggcattcgatagaagaaagaactatagacaactagtcttacaatatgacaattctctttgattaataaatgaaaataacacttgtgtcagcctaatagccgagtggcgggcatctctggcggcctcccgagcagcgtggaatgcgtccaagatcccgtccgcgggtcgtcctccggtcggaatgatgactggagcagcagacgatatcctgacctgaatgcatgtgatattcacattccagggagaattgtcggctatttagaaccctctcggcttaaaagccctattagactatgggtgcgctcaagccactagccaggaattcccgctgaacgctccatcaccttgcagctgaagtgcaacatgggacgggctttaacttttcgtagatataagtttaatctatcctctccacacccatagggtcgtatggcgtcaaccagggcactctgcaggatttcatctcgcttcgccaagcgaggcgccctaacgggcagcctgcagcttaccctgttaaccccggctcaccaccccccgagcaatccgtcgcgtcctccacgagtcataacaaggttcgggcgttgtttcttacccccactatcaggcgtattcagttaacagtcagtagtcccgtgtcggagatttgttgttctgcaacaattaaaggggaccggggttaaatcctggcccccgaactgatcggagtttcggccaatgagagatgttatatacgcccgttcctggctgatggattaattgccggctccatttggcatccatcaagcatcatacgggattagaagggtagttcgtgggttgatctgccgtgcaaggtgctcaaggctctggagtcatgctgaacgcaaatatttaagaatcgtcgtcagggacagcgttctctggatagtcaagctgtgcttgggacgctgttctgtcgctttgtcaaaacataatttgcagcgatgagattatcgacttcgagtctcttcctttccgtgtctctgctggggaagctggccctcgggctgtcggctgcagaatggcgcactcagtcgatttacttcctattgacggatcggttcggtaggacggacaattcgacgacagctacatgcgatacgggtgaccaagtacgttggtattgcaggacttccatcattcatctactgacttgaatagatctattgtggtggcagttggcaaggaatcatcaaccatgtttgtgatcacttcatactatccgctgtgcgcgtgtctgactttatttgctgcagctggattatatccagggcatgggattcacggccatctggatctcgcctatcactgaacagctgccccaggatactgctgatggtgaagcttaccatggatattggcagcagaagatgtatgcgctcctccttcccatatcgtaggcttactctcaggcggcgactgacttgacagatacgacgtgaactccaacttcggcactgcagatgacctcaagtccctctcagatgcgcttcatgcccgcggaatgtacctcatggtggacgtcgtccctaaccacatggtaagtgctgcttcagcatccttatcagtgaactccaagtgccaacgctaactgtaccagggctacgccggcaacggcaacgatgtagactacagcgtcttcgaccccttcgattcctcctcctacttccacccatactgcctgatcacagattgggacaacttgaccatggtccaagattgttgggagggtgacaccatcgtatctctgccagacctaaacaccaccgaaactgccgtgagaacaatctggtatgactgggtagccgacctggtatccaattattcaggtgcgaattccaacccaatttaaaataaccatatactaagtgaaatcaccagtcgacggactccgcatcgacagtgtcctcgaagtcgaaccagacttcttcccgggctaccaggaagcagcaggtgtctactgcgtcggcgaagtcgacaacggcaaccctgccctcgactgcccataccagaaggtcctggacggcgtcctcaactatccgatgtacatccccctatacattgttcattagatcttcgctaactccaaccagctactggcaactcctctacgccttcgaatcctccagcggcagcatcagcaacctctacaacatgatcaaatccgtcgcaagcgactgctccgatccgacactactcggcaacttcatcgaaaaccacgacaatccccgtttcgcctcgtatgtcccaccccctcccctccctacaatcacactcactaatacatctaacagctacacctccgactactcgcaagccaaaaacgtcctcagctacatcttcctctccgacggcatccccatcgtctacgccggcgaagaacagcactactccggcggcaaggtgccctacaaccgcgaagcgacctggctttcaggctacgacacctccgcagagctgtacacctggatagccaccacgaacgcgatccgcaaactagccatctcagctgactcggcctacattacctacgcggttcgtccttccctcccaccctttaccccccaccctacaaacatcccacatactaacaacatttcaataatgaaatagaatgatgcattctacactgacagcaacaccatcgcaatgcgcaaaggcacctcagggagccaagtcatcaccgtcctctccaacaaaggctcctcaggaagcagctacaccctgaccctcagcggaagcggctacacatccggcacgaagctgatcgaagcgtacacatgcacatccgtgaccgtggactcgagcggcgatattcccgtgccgatggcgtcgggattaccgagagttcttctgcccgcgtccgtcgtcgatagctcttcgctctgtggcgggagcggaagattatacgtcgagtaattccggagtggtcggttactgtgacgttgccggtggggacaacctttgagtataagtttattaaggtggagtcggatgggactgttacttgggagagtgatccgaatcgggagtatacggtgcccgagtgtgggagtggggagacggtggttgatacttggaggtagatggtttggtcttattgttttattaagtgtgatgagggtggtttggaatgtatgtagtttgggctttggtagtgttgggttgggttgggttaatgattttgttattgtattgtttttggtggttgtgaccatggatttgaaatgagattttgtaggggctacggaagtgtattgtggacatgtatgtgagttaattcatctgggtatgtacaaagttggttagccagtgggcttgaagaaaagtctcctgggtctctggtttgagtacccatgttaagagcaagcataaaaacatgaaatattgggaatacaaagggtatttaaaactcgtgagcattagctcctgggtagaatgcaatcataacagaaagtacagccagcgctgtgtcataaagaagtccagttgggaaacgaaagactagaatcaaactaaaagtaatccggccgatatggcttcacgtgcgaagtctcgccttgaggggacattgtccttgcaggtgattgaccattgcgttcatatggcgcgatgtttggtagtgtgggtgtagccggtgacctcacggaaggactgaaggccacatacccttctgagggcctcttttcttcgtggccggagctctcgaatgggttctcgacaggtacactcgtttggatgtggtcatttgaaggtctgcgttcggtcattgttcgcgcaggcgagctgactgagggattgaaagctgcatagccatcattggcatgcgttaattcgccaaagcttagcggcgaaacaggcctgacctctaacccatgcatctgctctgcactcgattgttcgtggtgtccttgcgaagaaagagaagccttggactcggatgactttctggacgaggtggtaggatcatcatgattgtaatgagactgtagcacatcatgcgaatcattcgacacacggtgtctgcccgagctgacgtcagcatcggtatgcatttcgatatgatcctcatggttctcagcatgtccctcgagaggggactcatttccagcggcaggattataagcaacataattgtcatgtggttgcgacctttcgtgagactccgagtttgatctcactgtggactcatgggcgatatgcggctcatcatgatcttcgaatggagaaaaatggttgaagtcggaggacacgggtgatttagcagcagggttgaatgcaacacagccggtctcgcgctcttcatgtgagctatatgagtcatgtggcctgtcatggtccagaggctccggatgctcatggctagattcatcgtgtgccgaaatcgcgtcactagcaaagggcgaggttgacacattggctgcaggactgaacgccacataaccactctcaggctcttcatgggagttataggagctgtgtggcatatcatagtcctgaggttcacgatgctcatggctggattcatcgtgtgccgaaatagcgtgactagcaaaaggcgagggcgaagcattggttgcaggactgaacgccacatagccgtccccattggccgaattgactggtgacaacgtcctacccatggcgtcggcgggggcagcggtttggtgagagcgaagaccatgagaaatagctgggctgaacgatcgcagttggtattcgttttcttgagctggatagggggctgcgtcaggctggctgaaaggtgagaatgttcgggttgctgctctatcaccagggaaggcagacgctggagtcaaagaacgagtgtttggatcaattgccggactgtatgaacggaaaggagtgctagatggagggccataaggatcgtaatgaggctgatatgtttcatatggcctgtagccttcgctaggacctcgtggttcggggaccgttggcccatacccaggagctggtgtataattggaacgcgacacgggtgtttgattgcgcagaatttgcggtgccggcgaggcgtgatcaatctggctgtaacctgggcctggggtgtagtttgagacaggtgtttgtgttcgtggcatttgtggcgctggcgacgctctgtcagtcggcccatatccaggcgccgaaggtgtg

SEQ ID NO: 28 - protein, 505 aa, Artificial sequence
  Synthetic Construct
MRLSTSSLFLSVSLLGKLALGLSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRLYVE

SEQ ID NO: 29 - DNA, 24 bp, artificial sequence
  Forward primer
cgtacaccttgggattatgcgctg

SEQ ID NO: 30 - DNA, 24 bp, artificial sequence
  Reverse primer
cacaaaggcgcaaagcataccatc

SEQ ID NO: 31 - DNA, 29 bp, artificial sequence
  Primer pyr-F
ttaattaaactaaatgacgtttgtgaaca

SEQ ID NO: 32 - DNA, 42 bp, artificial sequence
  Primer pyr-R
ctaccgccaggtgtcagtcaccctcaaagtccaactcttttc

SEQ ID NO: 33 - DNA, 38 bp, artificial sequence
  Primer Tamg-F
agagttggactttgagggtgactgacacctggcggtag

SEQ ID NO: 34 - DNA, 83 bp, artificial sequence
  Primer Tamg-R
gcatgcactagctagttgaagttcctatactatttgaagaataggaactcggaataggaacttcaacctagaggagagagttg

SEQ ID NO: 35 - DNA, 2143 bp, artificial sequence
  The A.nidulans pyrG gene with flanking sequences in pHUda794
attaattaacctagtactaaatgacgtttgtgaacagcccaaagcctacaaattcaactgcgcacaacgcgcccacggcaacttcctcgagaacgcgccgcagacaatgctctctatcctggtggcaggcgtcaagtacccagaggcagcagcgggcttaggagcggcctgggttgttctccgcaccctctacatgctgggctatatttatagcgacaagccgaacggcaccggcaggtacaatggttcgctgtacttgcttgcgcaagcgggtctttggggattgagcgcatttggtgttgcaaaggatttgatgtaaatgtagtcgacatcttagcacagaggggagagttgataaaatgtggtctgtttgaatgatagtcgggttcgtgacctatattcgtgatagtggagataggtctgcgcctatcttatcgggccggagcaaaaattccaccgcagcggggtgagttttcgttatacagccatcccacttccagcttcaaattgtcagtttaatccagcccaattcaatcattggagaaccgccatcatgtcttcgaagtcccacctcccctacgcaattcgcgcaaccaaccatcccaaccctttaacatctaaactcttctccatcgccgaggagaagaaaaccaacgtcaccgtctccgcagacgttactacttccgccgagctcctcgatcttgctgaccgcctaggcccctatatcgcagttctgaaaacccacatcgacatcctcaccgatctcaccccgtcgaccctttcctcgctccaatccctcgcgacaaagcacaacttcctcatctttgaggaccgcaagttcatcgacatcggcaacaccgtgcaaaagcagtaccacggtggcgctctccgcatctccgaatgggcacacatcatcaactgcgccatcctgccgggcgaagggatcgtcgaggccctcgcacagacaaccaagtctcctgactttaaagacgcgaatcaacgaggtctcctgattcttgccgagatgacgagtaagggatctcttgcgacaggggagtacacggcacgctcggttgagtacgcgcggaagtataaggggtttgtgatgggattcgtgagtacaagggcgttgagtgaggtgctgcccgaacagaaagaggagagcgaggattttgtcgtctttacgactggggtgaatctgtcggataagggggataagctggggcagcagtatcagacacctgggtcggcggttgggcgaggtgcggactttatcattgcgggtaggggcatctataaggcggacgatccagtcgaggcggttcagaggtaccgggaggaaggctggaaagcttacgagaaaagagttggactttgagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggccgggtggtcattggccgcctgtgatgccatctgccactaaatccgatcattgatccaccgcccacgaggcgcgtctttgctttttgcgcggcgtccaggttcaactctctcctctaggttgaagttcctattccgagttcctattcttcaaatagtataggaacttcaactagctagtgcatgc

SEQ ID NO: 36 - protein, 274 aa, artificial sequence
  Synthetic Construct
MSSKSHLPYAIRATNHPNPLTSKLFSIAEEKKTNVTVSADVTTSAELLDLADRLGPYIAVLKTHIDILTDLTPSTLSSLQSLATKHNFLIFEDRKFIDIGNTVQKQYHGGALRISEWAHIINCAILPGEGIVEALAQTTKSPDFKDANQRGLLILAEMTSKGSLATGEYTARSVEYARKYKGFVMGFVSTRALSEVLPEQKEESEDFVVFTTGVNLSDKGDKLGQQYQTPGSAVGRGADFIIAGRGIYKADDPVEAVQRYREEGWKAYEKRVGL

SEQ ID NO: 37 - DNA, 33 bp, artificial sequence
  Primer xln-F
gcatgcttaattaatggaagtgcgttgatcatt

SEQ ID NO: 38 - DNA, 19 bp, artificial sequence
  Primer xln-R
ggatcccctgtcagttggg

SEQ ID NO: 39 - DNA, 2477 bp, artificial sequence
  The synthetic version of FLP (sFLP) expression parts in pHUda996
gcatgcttaattaatggaagtgcgttgatcattattccccgaaaatgtagtacccagtaagtggtctagcggtggctatggtaggacatctatgcctaagctggagttctcattgaacgtgtaccggccgattgccctaaactctgattgagagccggaaacctcatctacctgatgctcaggggccatccaatagcttccgatagcattacagacagatggactcgtcttggcccacgggtctagaacagtcgccggaactgcctctatttgaaacggagctgaaccatgatacttaagcgtgccaagcggcgccgtttcccactggaacaaggagcaatagaattctgcagagattcttcattcaggctattcagcaattcggtttgtggagcggatcggggtccactgggtttagtctggggtttttctttgcccgcatgggctctagcacatgcacagcttgcagttgctgctacgctatctgggaaaacgaatggctattcaggagtttataaccaaaagagccggaaacaggctgattgccctctcacggggagacgttgtacttctgatccagaggctattaaccggacactacctataaaggaggtagcattcctttctgtccggctcccagattccaacaacccaactgacaggggatccaccatgccccagttcgatatcctctgcaagaccccccccaaggtcctcgtccgccagttcgtcgagcgcttcgagcgcccctccggcgagaagatcgccctctgcgccgccgagctcacctacctctgctggatgatcacccataacggcaccgccatcaagcgcgccaccttcatgtcctacaacaccatcatctccaactccctctccttcgatatcgtcaacaagtccctccagttcaagtacaagacccagaaggccaccatcctggaggcctccctcaagaagctcatccccgcctgggagttcaccatcatcccctactacggccagaagcatcagtccgatatcaccgatatcgtctcctccctccagctccagttcgagtcctccgaggaggccgataagggcaactcccattccaagaagatgctcaaggccctcctctccgagggcgagtccatctgggagatcaccgagaagatcctcaactccttcgagtacacctcccgcttcaccaagaccaagaccctctaccagttcctcttcctcgccaccttcatcaactgcggccgcttctccgatatcaagaacgtcgatcccaagtccttcaagctcgtccagaacaagtacctcggcgtcatcatccagtgcctcgtcaccgagaccaagacctccgtctcccgccatatctacttcttctccgcccgcggccgcatcgatcccctcgtctacctcgatgagttcctccgcaactccgagcccgtcctcaagcgcgtcaaccgcaccggcaactcctcctccaacaagcaggagtaccagctcctcaaggataacctcgtccgctcctacaacaaggccctcaagaagaacgccccctactccatcttcgccatcaagaacggccccaagtcccatatcggccgccatctcatgacctccttcctctccatgaagggcctcaccgagctcaccaacgtcgtcggcaactggtccgataagcgcgcctccgccgtcgcccgcaccacctacacccatcagatcaccgccatccccgatcattacttcgcactagtctcccgctactacgcctacgatcccatctccaaggagatgatcgccctcaaggatgagaccaaccccatcgaggagtggcagcatatcgagcagctcaagggctccgccgagggctccatccgctaccccgcctggaacggcatcatctcccaggaggtcctcgattacctctcctcctacatcaaccgccgcatctgagtcgagattatccaagggaatgacttaatgagtatgtaagacatgggtcataacggcgttcgaaacatatacagggttatgtttgggaatagcacacgaataataacgttaataggtaccaaagtccttgatacattagcacggtagaaaaagaataatacaacgagctgggaatattctttaatataaaactccaagaagagctggtgcggtggagcttgttttcgactctcagtaatatttcctcatatccaagcgcgctaggaggtggtcgaatacacatgtaggcgcttctctggatgcaaaagtcgtgccggacctgccgaaagactttgaagatgcgttcacgccatctaagttgcgtagataattcacaaaaagggatgtttgtttccggaatgtagcaaagagctgataggcaatagcctcactttcgtggcgcacgccgctcgttccatccatcctcgacaatggagcaaatgtcaaaatcgtaccgaaaatactttgctagcgaagttcctatactttctaga

SEQ ID NO: 40 - protein, 423 aa, artificial sequence
  Synthetic Construct
MPQFDILCKTPPKVLVRQFVERFERPSGEKIALCAAELTYLCWMITHNGTAIKRATFMSYNTIISNSLSFDIVNKSLQFKYKTQKATILEASLKKLIPAWEFTIIPYYGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYSIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 41 - DNA, 81 bp, artificial sequence
  Primer Pna-F
gaattcatcttgaagttcctattccgagttcctattctctagaaagtataggaacttcgctagccgagagcagcttgaaga

SEQ ID NO: 42 - DNA, 29 bp, artificial sequence
  Primer Pna-R
ggatcccccagttgtgtatatagaggatt

SEQ ID NO: 43 - DNA, 24 bp, artificial sequence
  Forward primer
tcgagtgcggccgacgcgtacgtc

SEQ ID NO: 44 - DNA, 20 bp, artificial sequence
  Reverse primer
cagagagtgttggtcacgta

SEQ ID NO: 45 - DNA, 33 bp, artificial sequence
  Primer 3ku-F
actagttctagaagccgtgggtatttttatgaa

SEQ ID NO: 46 - DNA, 34 bp, artificial sequence
  Primer 3ku-R
gaattcgtttaaacttggcggctgccaagcttcc

SEQ ID NO: 47 - DNA, 28 bp, artificial sequence
  Primer 5ku-F
gcggccgctcattcagagagctacccgt

SEQ ID NO: 48 - DNA, 30 bp, artificial sequence
  Primer 5ku-R
actagttaattaagaggaccgcatctttga

SEQ ID NO: 49 - DNA, 6133 bp, artificial sequence
  The A.niger ku70 gene and flanking sequences of pHUda801
cacagctcattcagagagctacccgtagtagaacaggaatactgggggtattgcgaaaacgcgaccgcacgaccgcccttcccattgccaaaaccatcttccagcaattgtgtgtacatttgttccgtcagcgggttggcgtagcggaaggcaacgtacggcttgtgaggcgcagtctccgggttgatcttgtccagcagcttgcacatttcctcgcattggtattccgaccattttcttatgggtgagcctccaccgatgtccgcatactgtttttgaatcttgggtgtgcgtcgtttcgaaataagaggcccgaggtaatgctggaacttgccaagaggaatcaaatcgccgtcggccttgaatagaagtagaatgttagaaacggagcaaccaaaatgacagcttgccatagtcggagacgtacaaagagccggctgaggaaatcttctacttcgtctgtcgtcgagggccctcccatgttcaggaagaccatggctgtagggcccttagagcctgttgcatcctgggtaaccggaggcactgttgtcgccagcccacatctttgttcttgcttgtatccgaacagggtgcgagaagccggtcgcagcaattgccggggaagggtaaacgggcggcggagagccatgacaggtaattgtactgaattcggttgacctagtcaatgggggtataagaaaagaccgttcgtatcgcgcaagcagatgaactattcaagcccgcattcaatacttaaaagatagacgagtggcaagaacaggtagtgggtgtatgcaacagcgcaaggccttctggaagctgaaaagtccagaacggcttgatgacggagcaccgagaccacgaccaactccgactcccgacagccaatgaccggccagctagcgtcatcaattaccgggcggacatcacatgatgttcgtgtctccccgcgtctttctgcccaccggtttgatcgcgtccctcgcgaccggatccagtgacgatatagatctcccctcggctgcaggcagcagaggccaaacaggcagacacaacagccccacttgttcctggttacgattcaagttgtcttaacctttatacttccctctttcaatttcgataatatcttgattgctttaaacgattccacaacattctactatggcggacggtaacccacatcgggaagatgaggcggccgaggaagaagaggagattgatgagactgtacgcaaatttacccatgaacttggactggaactctggaactgacaataagatcagagctacaaaccagtcaaagatgcggtcctcttcgcaatcgatgtcagcgattccatgttgacgccgcgcccctcggcagatcctaagaaacacacccaagaatcacccaccacggcagcgctcaaatgcgcctatcacttcatgcaacaacgaatcatatcaaatccacaagacatgatgggtgttttgctgttcgggacccaggcgtccaagttctttgaagaagatgaagacagtcggggagacctgtcctaccccaactgctacctcttcactgatctggatgttccttcggctcatgaggtcaaagaacttcgagcactggtagatgatgaaggagactcaagggaggttctatctccagcgaaagagcaggtctctatggcaaacgtcctattttgcgccaaccagatattcacatccagagcgccaaatttcctctcccggcgtttgttcatcataaccgacaatgacaacccccatggtgatgataaaaccctgcggtcagcggcgactgtacgtgctaaggatctttacgatcttggtgtcacaattgagctgtttccgatctcacgccctgagcatgagttcaagaacagcaagttctatgacgtaagctatcatactctatagcaaagtggcaggggtcgatactcactacagatacaaaggatattatctacaagtcattgcccagcgatccagaggcgcctgcatatctacaatctgattcaaaagcggcgactgcgaccggggacgggatttcactcctcaacacgcttctgtccagtattaattcgagaacggttccgcgtcgcactcatttttcgaacatgcctttagaacttggcccagacttcagaatttcggtatcgggctatatactcttacgaaggcaagcgcccgctagaaactccttcatctggctgaacggcgagaagcctgtggtcgcgaaaggagtgacttcccactccgcagatgatactggccggactgtcgagaaatgggagatcagaaaggcatataagttcggtggcgaccaagtaaccttttcgcctgatgagcagaaggcgcttagggatttcggtgagccagtaatccgggttattgggttcaagcctatcactgcgcttccattctgggcaaacgtcaagcacccatattttatctatccatccgaggaagactatgtaggctcctcgcgagtattttccgcattgcatcagactcttttgcgttccaagaagatggcactcgtctggttcattgcgcgcaagggtgctggccccgttctcgccgctatgatcgcaggcgaagaaaagcttgatgagaatggcgtacaaaaataccctcctggcatgtggattcttcccctccccttcgcagacgatatccggcagaaccccgaaacaacgttgaatgtcgccccggagtcattgattgatcagatgcgcgtggtcgtccagcaactgcagctgccgaagggagtgtacgagcctctcaaataccccaatccatgtaagtcactgctgtcttgcattgctcgtatacgatgaacgagaagttgacagcccgtgatcagcccttcaatggcattaccgcatcctacaagctctcgcattagacgaagatctccctgaaaaaccagaagacaaaaccattccgaaataccgccaaatcgacaaggtaaaaccactacacccaagaaacaaccctccacgcattcaacctactgacaattgcaccgcagcgcgccggtgactacgtattatcctgggccgacgaactcgaaaagcaatacgccaaaacctcagcagcggcccctcgcccaaccagcaccctcgtgaaacgaggatcaaaagaccgagcaagcgaaaccgaggactccaagccatcgaaaaagatcaaggttgaggaagactctggaagcctagaggaggaagtccgcaggcatcacaagaagggaacgctatccaaggtaagccaccacaggctttctacacgtcctcgtgatggcaaatatgacatcgtattaaccggcggttttctagcttacggtcgctatcctcaaggacttcttgacttccaatggacgctcaaatgccggtaagaaggcggatcttattgagcgggtagaggagttcttggagcagtgacatggcgggattgttggattcgctagtgcgcttctgttggtggatgtcgttatgtggtgtcttatctcgggttaggcgttcgtgacctgaggacatgagcttgtaattaatgatgggttggatgtcgcggtattcgttcttcagcgaaacgtaatggacacgtattttaggcgatgtacagttataaaaatcgaattcgctgggctagccggacatgtcaaaacgaagagtattaggagagacatcaggtccaagtgctatttttcaaaccagtcgcttaagaccaccgaggcctttatctccagaaaatataccggttcagcaggtgcgcgtatcccgaattcaaattaatattggaacgatcgtaaataaccgcccagattcgccgtaaaacgatagtagtcaggctttgccgccgacagaaggggacgagtatgtcaactgagtcaacttgaaccgagcagcccctctaaacaacgccacgctgtttgtaatatccctttagaaacgtgttgtcgctggcaattatccacaaaaaatgagtctaaacgggcgaaaaaagtcaccaaaatgggagaatatgtggaaagaagaaagaaagagagaccaaagcaagagagcgccgaaaggaagctatcgtaatatatacaagtagaagccgtgggtatttttatgaaagcagaaacgttaacggtatgcgtacaatgatcaacattgtccataaacttgacagtagcagacttcttcgtcgggacagctgagagtagcgaagtgttagtatttaggacgcattcagcaggtagacgggggaggtgtgcaaaggcaacatactatattgattctttgccgaatatgacatgccagagaaattccatgacacggccactactggcgtcatccttgtcggtatcgattatccactggcggatcttgatgtagtcctctcgtggtcgttggtggacctgctcccgggacacggcgaattgcgcacagcacgccgcgccaatctgtttcggcatttgcaggaacttctggtatttagcttcgtcgtattcatcttgcatcgtaaggcccccggtggagttcaaaggcggggtgctggtgccctcaaatatctctgcaaagacttcttccgtcacgtgctggttggtgcgatggccctgcttgcaccccgggttccagttgcagcggaggttgacatagccattgtcttggacgaagttgagacgcagggatttcatcgcaatgacattgtcgtgtaagggcgcatctacatgccaggccatcaggaaacccgagcgatgggaatgaaggaaagcaatagtggatggtagggtgtcgtagtgatcaattaggtaggtgagataagccatggactcgtgacccttgttcagcggggttgtgagtgttgtgccgtcagctgcgacctttttggagggattcacgatgtatatggcacgttgccagctgtggagggaagctattagtgcgccgaaacattgggttgggaaggggaggacaaaaaaaactcactctggtagctcctgctcgacccattcagtgtgctcttcctgtagcctggccatcacaatcactttatcccctggtgtgacaggacgagagccatttaatgtgttcatcggcgggcgcaaatccagccaattgatcagatatgcgcccgcgcgatactgatcgcaaaggtcctcgaggtggatcttcaagagatataagggcaagatgagtgctagactagcacatagtgctatgcgagcgccccatctcatgatgaatggctaaaggacggtagcttctgtgcacggtacgggactgtttccagaagaattggtgaaaacgccaacgaggaccaatatcgaaaggccgttatcgatgtaatgcaggttaaaatctgttcctctcttctgcaggtgacgaaatcataggactatgaaggtggatgttgtcacaacacgggttggtgtaaggaatgacttgagtcgggaaagaccgagaaagatgaaagacggagaggatagtttcgagcattaaaaggagggtcaatcctattgaggaagaatccaaaagaaggaaaagaggaaaaatcgaaaaaggagggtaggtcttggaggggctatatgtgaaccatattgcgagaaacagaggacgagagtaggaagagagaagacggacgagaggggtcaaggcgaggtaatcaggaaaggagactggaggaccgttgattaacctattcgtctgcctaccaaggcggtaggtgcagtagtaggagaagtagtcaaggcaccaggtacttttagtgactttggactaaaatagtcactaagacgagtaagttagtggtggaagggaaggtccatcgaatgcttccaattcaagcccccatctgcctccttctgtttcgcccgctctcaggcacttcgaatctttcaatccgtctgtctgtctttttgggtggcgcggcatgggggcagtggggcgtaaccggaccccccactggctggccacgcgttaaaattgttgctatcgtttggctccccaaagatgaagactaagggggaatggaagaggctgtcataggctgttgagagacggaggtcccctcagacaatcgcaaaccttaagggaaccactttcttccttagcgggccttagcggggaagcttggcagccgccaa

SEQ ID NO: 50 - protein, 653 aa, artificial sequence
  Synthetic Construct
MADGNPHREDEAAEEEEEIDETSYKPVKDAVLFAIDVSDSMLTPRPSADPKKHTQESPTTAALKCAYHFMQQRIISNPQDMMGVLLFGTQASKFFEEDEDSRGDLSYPNCYLFTDLDVPSAHEVKELRALVDDEGDSREVLSPAKEQVSMANVLFCANQIFTSRAPNFLSRRLFIITDNDNPHGDDKTLRSAATVRAKDLYDLGVTIELFPISRPEHEFKNSKFYDDIIYKSLPSDPEAPAYLQSDSKAATATGDGISLLNTLLSSINSRTVPRRTHFSNMPLELGPDFRISVSGYILLRRQAPARNSFIWLNGEKPVVAKGVTSHSADDTGRTVEKWEIRKAYKFGGDQVTFSPDEQKALRDFGEPVIRVIGFKPITALPFWANVKHPYFIYPSEEDYVGSSRVFSALHQTLLRSKKMALVWFIARKGAGPVLAAMIAGEEKLDENGVQKYPPGMWILPLPFADDIRQNPETTLNVAPESLIDQMRVVVQQLQLPKGVYEPLKYPNPSLQWHYRILQALALDEDLPEKPEDKTIPKYRQIDKRAGDYVLSWADELEKQYAKTSAAAPRPTSTLVKRGSKDRASETEDSKPSKKIKVEEDSGSLEEEVRRHHKKGTLSKLTVAILKDFLTSNGRSNAGKKADLIERVEEFLEQ

SEQ ID NO: 51 - DNA, 21 bp, artificial sequence
  Forward primer
acggtatgcgtacaatgatca

SEQ ID NO: 52 - DNA, 21 bp, artificial sequence
  Reverse primer
atttgagggcaccagcacccc

SEQ ID NO: 53 - DNA, 24 bp, artificial sequence
  Forward primer
tcgagtgcggccgacgcgtacgtc

SEQ ID NO: 54 - DNA, 20 bp, artificial sequence
  Reverse primer
cagagagtgttggtcacgta

SEQ ID NO: 55 - DNA, 30 bp, artificial sequence
  Primer 3fcy-F
tctagaattgaaagctagttctggtcgcat

SEQ ID NO: 56 - DNA, 32 bp, artificial sequence
  Primer 3fcy-R
gtttaaactccttgcttcgcatacatgcccac

SEQ ID NO: 57 - DNA, 30 bp, artificial sequence
  Primer 5fcy-F
gcggccgccgccgccgaagaactgagcaaa

SEQ ID NO: 58 - DNA, 29 bp, artificial sequence
  Primer 5fcy-R
actagtatatcttcttatcgcagagattg

SEQ ID NO: 59 - DNA, 5230 bp, artificial sequence
  The A.niger fcy1 gene and flanking sequences in pHUda1043
ccgccgccgaagaactgagcaaagaggtcctcggcgcccatgccaccgccagcaccgccatgctcaagaccctcctcaccgagctggtcgtagaggctacgcttctggggatcggagagggtctcgtaagcggcagacaattccttgaacttctcagcggcttcggggttgtttgtgttcttgtctgtaaagatatcgcgttagtaaagacccctagatctttcgtgaaaagcaccgctttcgcgattcaagttgacttaccagggtggtacttcagggcacccttcttgtaggcagtcttgagttgggcctcagaggccgtcgggggaacctaaccgcctcaccgttagtttctgtgacgtgcaaaaccagccaaacttggcgaaaacccagaacatacccccaggatgtcgtagaacttagtttccttgaccattgtgatctgtgtctagaagagagaaaaaaatcgaaaggcgaaagttgggcgacggggagaagccgagggaaaatatagaagaaacaagaacttttcggagggacgagacggggcaatccgatcctagaaaaccttacaccggggtatggaacaggcgaaacaaagagggctcgaaaccaaggagtgtagagaaatccttgaaaaagagggaggagtttgaggagacgaggggagaggagtctcgaaggcgtgaggggggacaagtaagaggtggaaggaagaaggaagagttggagagagagagggtccgtccgggtgataatcaaagccaggagagcgagagagagaagagagaaagcggcgacagggcggcggctgagacaagtgagagggtatcgtatgtgtaatctgattaccaggaccaggcaccaatcgacctttgatttgccgcacgagcgcagtgaagactcgccaagagtttcaagatgggcgtatcaaataccggagatacgatggggcgaaactccgggagtatagaaatgctggagaagatgagcgaccgcgcttcaactggctggaactggaattatatagaaaaaggtggatagtggttctgaaaagatcagatcttaacatgaaaggagagatcgtcgtatgcttttgaacaaattggcatgtccacgatgacaacgtctcaggctgaatggagttgttctctttgcttcgacaagccccgtcacccgcagccttgatggcccgagcgtggcttccgactgcttcgatgcgattccgtgtccttccaccgctttcgcaccctttccatcgctgacaatcgccctggtaattaccggttcgtgaccacggagagcgttggttgcatcccatttctggatgtctgaccaaatgtatcaccgggacttttctatcttgttcgatactacagtagggaggggtggtcctaagtaagctagttctttcatgcctcggtaggatgcggcaaggtctacctgggtattacggtccaatcatacaccattcacggggatcctcgtcttcatactactactactaagtactactacttactgcggtgtattgtgtagcatccctccatccaccatcactactcatcatcttacatgtataaaatacctacccagtatattacacccggaaactccaagcacaaaaaaagaaaaagaaaataaggaaactgtaaaattaaagtttgatgtagccgcccggatctgcctttgtctcccaagtcagattcttttcttcctggcacacagcgccatttgcctcaggcacttggaattgtggcgggcggtggttgattgccggcttatcgataaggagaggcgattagctcgatgcggaaggaggggaagaaaaaagcctgtcgggattccccacctggagattcgtcgcgatccccaattgccggctggcttcggttcaactggctcatgcctggtgtactctactgttcttctgctgctgcagaggcaattaatggttcaattccggagtaccagacagaaattggcttctgtggttcttgccatgatcgtggatcagaggttcagagaacaatctctgcgataagaagatatactagtaaatagtgcgtggcatggggtctgcgcgagatgaaccccgagtttatcagccactgccagtttgatcttgtaaattgtgaaactgtgaattaatggttacaagtgataaggacgttaccatcgggctctatgcatctagatcggatgtctcatatacaatcagctcaatttgtattcagttatagttgtatacaaggcatgaaatattaagcatctttcttacgcttatgcatgtcgatccccaagcacaccaaagaagcactttatgcataccataacccaagaaagtctatcacatgcacacattatccatgaaaatactattcaatacgaatgtaacaacgtcctctatcagtctcaatgacagcagctatcttgttatcatggagctccgcacgtccagcccgatgcgggtcagtccggcagttaaccacacagagtttgctccgtcttgatgctaccccatctttctatctctctcccaattacccctccaatcgctctatatttcatatctcaatacagcatacaacaagcacataccatcatggagaccgatcccggattcatcgctgctgtggaagaagccaagcaaggcgctgctgagggtggtgtgcccattggagcttgtttggtctccaaggatggcaagattctaggccgcggccacaatatgcgcgtccagaagggtagtcccgtgttgcatgttcgttgatcccatcccttgccttctgagggtcgtctggggttctaattctaatctctaccgtcataggctgagatgtccgcgctcgagaactccggtcgtctgcccgcttcggcctacgaaggcgctactatgtacacgaccctgtcgccatgcgacatgtgcaccggtgcctgcatcctctacaaggttaagcgcgttgttgtgggcgagaacaagagcttcatgggtggcgaggactatcttaagagccgtgggaaggaggttgtggttttggataatgcagagtgtaagcagctgatggagaagttcatgaaggagaagccggagctttggtaggtttcccatgcatctcactggactggtctagtcttttgttggaatgtacgctgactgtacgatgtctttgcaggaatgaggacatttccgtctgagcttttgaattcgtgaaggtgtcaactatattgctggctaggctctcatgtacataataaagaattgaaagctagttctggtcgcattgagcacccaatttagaccgtcagacggtggatctcttcgaagaagaacttgagatcatccgggttgacgaaaggagtcacacctgtgatacattagcatttattgaataacccagctgtggcagtgctcaccgtgacccaagttggcaatccagccttgcttgcccttctcgaatccccgaaccatagtctccacagcctccgtgatagcctcgcgtcctccatagagaacaccagggtcagcattaccctggatcgtcacacgaccattggcaatccgcctagcctcagcggggtcgtgcagccagtccaagccaacaacattgtagcccgactcgcagagatcctcaagaccaaaccacgcacccttcgcgaagactgtcatcggaacaggctccagacccatctccttcaacttcttcggcagattcgccgaaatgtgacgcaggtagggaagagagaatgacttgaaagcggccggagacagctcacccgcccaggaatcgaagacctgtaccagctgagcaccagcagcaacctgaagcgccaggtattcaacacagatctcggcgatcttctgcaggagagcctgcgactccttggggtacttgtagatccacttcttcgactggacgaacagtttcgtgccgcctccctctaccatgtagcacagcagagtccacggggcaccgcaaaagccgatcaacggcacacgaccctgcagcttgtggcgggtgagggtaatggccttgtagacgtagtcgagctccgacttgacatccacatccttctgcatcactttctcgtactgtccatcatcgggcgactggagcggctcggggaagtggggtcccttcttgtcgaccatctcaacctgcattcccatggcctggggaatgaccagaatatcggagaagatgatcgcagcatcgatgagtccggcgtagcggtcgatgggctggatcgtcaacgtcgatgcgacttcggggtcgcggcagcattcgaaaaagtcgcggccggctttggcttcatggtattcggggagataacggccagctatataagaagcacaatgtggtcagttataagagagacatacttggatccgggactcgtaagagtgcgaagagagtagtttgtagagagaggcgtaccttgccgcataacccatatcggaggacgctggactttctcgcctgcaatgatatcagtcccatctctttgaatataatggtttaaaatcagaattacccctagcagccctcagcaagaggtcgttcttcaatggctcgaattgatgctgcattttgatgtgggatgtgtgagtgatggaggggaccttgcggaggaggggccttcgacatgcaagtcctgccaccgttcgcggcctcgggccggaactcgactggtcgtccgtggctcaggtaagcttcaagccgttcgcaagtctggaacatctgcttactctacttcgattaagatggcataatttacgcagctcgagaataactatgaggcaatgcgatgttgattttattgacatgtatgtgctattaagtaccgagaatattcctccctcgcgtcccgacagcgacgaccaatacaatgccccacaaatccttcgcaacaaacaacagcctcaagtactacgctcttctagtcgctacttgaacacaaaccccaggacaagcctctgaggtaatgaacagcgcgacggccttacgtccggggctacattgaatctgtgaacctgaaagctgttagctatcagaccattgaaagtaatgcgtgacaaatggcaagttgaatggaatggtgtcagcaaaaaattagagtgtttgttgttgctgtttttgttcgtcaaagcaagtagctctgggttaaaaatgtatcatcatatcaccggggagtgggcatgtatgcgaagcaaggaaaaccaaatgat

SEQ ID NO: 60 - protein, 168 aa, artificial sequence
  Synthetic Construct
METDPGFIAAVEEAKQGAAEGGVPIGACLVSKDGKILGRGHNMRVQKGSPVLHAEMSALENSGRLPASAYEGATMYTTLSPCDMCTGACILYKVKRVVVGENKSFMGGEDYLKSRGKEVVVLDNAECKQLMEKFMKEKPELCSLDWSSLLLECTLTVRCLCRNEDISV

SEQ ID NO: 61 - DNA, 26 bp, artificial sequence
  Forward primer
gaaagctagttctggtcgcattgagc

SEQ ID NO: 62 - DNA, 24 bp, artificial sequence
  Reverse primer
gaagttgaaggagatgggtctgga

SEQ ID NO: 63 - DNA, 85 bp, artificial sequence
  Primer 3na2-F
tctagattgaagttcctattccgagttcctattcttcaaatagtataggaacttcatgtctccatgtttcttgagcggaagtact

SEQ ID NO: 64 - DNA, 30 bp, artificial sequence
  Primer 3na2-R
gtttaaacgaagactgatattatggcggaa

SEQ ID NO: 65 - DNA, 30 bp, artificial sequence
  Primer 5na2-F
gcggccgcaagagtcaaaagatagcagagc

SEQ ID NO: 66 - DNA, 79 bp, artificial sequence
  Primer 5na2-R
actagtgctagcgaagttcctatacttgaataggaactcggaataggaacttcaagatgaattcgcggccggccgcatg

SEQ ID NO: 67 - DNA, 30 bp, artificial sequence
  Primer fcy-F
gctagcgcgaggctatcacggaggctgtgg

SEQ ID NO: 68 - DNA, 30 bp, artificial sequence
  Primer fcy-R
gctagcttctgtggttcttgccatgatcgt

SEQ ID NO: 69 - DNA, 12700 bp, artificial sequence
  The A.niger NA2 gene with flanking sequences in pHUda1078
gctcagcgatacttcccgggaaaatcaagagtcaaaagatagcagagcttgaagaacgacttcggcagtttgctcttaaacgcgagggatcgaaaacattactgtacaacacaaagaaagaccttattagactccgcgctgagaaagacagtgtcaaaggagaaaaagaacgcctcctgaaggaaagagctacagaggagacatggtggtcttacatctcgtccttaatgataggaaacacggtggaattcaaccagcggagacaacgacgagagcgtgagataactgactcgattgggaaacaacggacgaaggaatggaatattgatcttaaactggcggaggttcaatatcttgaaagaatgctcgattctatctcttctgctgagattgaaatcaaagttgagataacgaaaatagaagagcgctggcgcgaaaggctatcattgcaggaaatggaaagggtattggcgaaatggaaaaatcaaagataattagcgaagggaactcgagtagcaacacggcatagatctacgaaggcagaaactatagccatcagtcatatattcaaaaaattgtggtagagtatagcgaagtgtgctaagtggtgccaactgaagaataatcagtggcaggaggaactttggtggatttgggacgaaatacacacgtggtaagaaatgtccttgtatgagaggatacaagcgacggaagccgcgctgagtcaccccagtgcatagttacgttttaatacagaagctggtaacagatgtccggaggaatagtcgtaaaaaagcttagcctaatcccgattagggcttctcaaacataggaagagtataaacatttgcgccatttttgcaacctagtgtaaacgaatggaatcaaaaaacacatgtgttaagccatccaccaagtcgtaaactcattatatggcccaagttcgttgaaccgtctgctttcacatcaacctcctctatgtctcgaagaatattctctatgtattcacctgcaccgctaagtttcattataagtgcgatagcacgatgaacatcaaggagccgccgtgagggcgtatcaattacacgagtgggactaagggttaaagtccgcgtgactgggaagagtggatcacgtaaaaatggacttcgctctgttgaatcaattttgtactgatatggcacgcctgtgggttcgaaataaatctgaaattcaccgaacatacgatgatagtcgagcgttaaagtaagggcgttaatggggctgtcaatcttcggaccatcaattagatggatgacaccagggtcaaacatatctaaaatccgaagcacattctttttcgagtcgctctatggaactatgttagcggagcgatgaagcatgttttagatcagacatactaggtctgcatctccagaggaaactgttgtaagacaatgtggtagaatatgggccacttccaggaactgaaagcggtcacttgattcgtttttcaattctattccttcatcgtccttgcaatcttccccatactgctcgaaacgttttctagcctcgctcttatcaaattttcgagaaattacgcaacgatagcgatcacgcacaaggcaactttttcgcaagatagatacacggtatggtgtgccggagggcgtagatgtttgtattgcggataatgatgcaggcgtcggttgcggagtcttgacagatgaagctcggactgtgatcatccaagttaaatagtgcttctcacagtagaatggtcaatagccaacatacgcggaaggaggaaattctcaatgatgtaatcagcgaattcttcgatcgcgctctttgctttgttcttctcgtctggactccaagacgtaaagttatcgaagaacgtcaagacaatcgtaatatcagaatcaatcaaatcagcaggctgtgaacatagattttcgtatatcgacgagaaaaagaacgttaaaaatgtatccttagctaccacatgctcatatgtggccttaataagtgccgctggcttgtaaccttttcgtgcactcctttcggggccataacactgaattaagacctgaagaaggttggctgccgattgactttggtggggtggtaatgagaaaggttttgagaagttcaggactttttctaaagatgactggtgtcgatgcaaaggatgtgaaggcatcatcgataagcccaatccattgtgtgaggtgtgcagaggaagccaaccaaggatgtttctacaacgcgcctcaggtcacgtggttgggagatcgtcgggttcttgtcaagtcgagaactgttaaaaagttagttgctcatgtacccgctagtcccacttaagactgtatcgttatcggtttatataataaatcttggatgactgtaacaatatatatatatattccagtagttaattgggctagtgacgggttaagctatggaacaatacgatcgattcaacgcgctttggtctcagtcatgtctgtattggttggcccaatctctaatgtcgctaacgaaccggcgtgcgacaccaattatcaccccttgcttgacgaagaagtctgggtcttccttcgaaacctgttgaaggtctaatccattttccaacgccacatcacgtgcttttttaatatggtctctataaatctcacggccaacacgcgacaagtgccaattggcatattcctccactgcatcatctaagaacccaggaatatcaacagagtcaatacagttgggacttgagcttgactgctcagccgtaacagatctgtctgttaggctttgcgatgactgcgcggggaggacattgatattgattggtggacaaattgatccacttgcggaatgcttcgggttcttttgtttttcaagccgcagtgcctcctctgcatagagttgttcacggacatcgtcaggaatatcatcatgcgtatccaagatgcctccaccctcaacgtatttgacaagtctcctcagatggtgcgttctaagtttgtaatgcttctttccaacagggtccagccagcaatactgcccttcatggcggcagggtggcccaggacaacgcatcgtttgatacacttcccgccaatatggacgttgtccagaagcctgttcagcatcgatctgggcgtctcgttctgtaagcattctcctagttactgatgactttcctctcttatctgtattccgtgaaagaggagggccactgtcctctatatagtttatggatataaaaagtttgagcttcttgccaatatgaaacagatttccccacattaagagctgtttctctataggtttccaatcaatattagtgccgtcaaaacgtttgttcagatcagattgtccacgttcgtttacagatactctgactgtagtatcatctgatctcacacgttggttgtgacgtatttttcgacgcataacattttcagcatcctgtgttatcttcgcccagtgtgaactgggtgctacagccaagtcctgttcagtgtcctttgacacagttcggttgttcagagttaccttccactcaatagtataatgaatacaaggctttcctctatgttgcctcgtagtcctttcttcgggctcctggaagaaacccagatgattgggctgggattgatgcaagggagtataaggttcatcaagtacatgttcaggtgatgggcaaaatacggatggcgtacgatctctaccgaagtcaccaggggtgggggcatacgatggagtttgtatccacggatcaggtggctgaagctgagaggcatcgtcatcgtagtaaggactaaacgtcatcccctcaaggcagtagatgccactgagaagcctagtgttgggatcatcatatgttagcctacaccatatgggtgtcccagcaagagtgtccgtgagggaagaggtgcagctaacaaaaccagtaaaatgatcaggttcatggacaatgaactaagacaggtacagtattgtagccctacccgtcttggttaacctggtaaggtcaaaaaggatcgaaccgtggctcagtacaaacaaaaggaatgttaacagtttgcgggagatgcaaggcacatgctttgtcatgtttgacgcgtttgcagtgtagaagcttccagctaccgtagattactgatacaaactcaatacactatttctataaccttactgttcaatacagtacgatcaaaatttccggaatattaatgttacggttaccttccatatgtagactagcgcacttggcattagggttcgaaatacgatcaaagagtattggggggggtgacagcagtaatgactccaactgtaaatcggcttctaggcgcgctccatctaaatgttctggctgtggtgtacaggggcataaaattacgcactacccgaatcgatagaactactcatttttatatagaagtcagaattcatggtgttttgatcattttaaatttttatatggcgggtggtgggcaactcgcttgcgcgggcaactcgcttaccgattacgttagggctgatatttacgtaaaaatcgtcaagggatgcaagaccaaagtactaaaaccccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtattgtcctgcagaatgcaatttaaactcttctgcgaatcgcttggattccccgcccctggccgtagagcttaaagtatgtcccttgtcgatgcgatgtatcacaacatataaatactagcaagggatgccatgcttggaggatagcaaccgacaacatcacatcaagctctcccttctctgaacaataaaccccacagaaggcatttatgatggtcgcgtggtggtctctatttctgtacggccttcaggtcgcggcacctgctttggctgcaacgcctgcggactggcgatcgcaatccatttatttccttctcacggatcgatttgcaaggacggatgggtcgacgactgcgacttgtaatactgcggatcaggtgtgttgttacctactagctttcagaaagaggaatgtaaactgacttgatatagaaatactgtggtggaacatggcagggcatcatcgacaaggtaaattgcccctttatcaaaaaaaaaagaaggaaaagcagaagaaaaataaaataaaaagaactctagtcctaaccatcacatagttggactatatccagggaatgggcttcacagccatctggatcacccccgttacagcccagctgccccagaccaccgcatatggagatgcctaccatggctactggcagcaggatatgtaagtcgatttctttaaatatctacctgtcatcttttacatcaatatgaactaacttgatggttttagatactctctgaacgaaaactacggcactgcagatgacttgaaggcgctctcttcggcccttcatgagagggggatgtatcttatggtcgatgtggttgctaaccatatggttcgtggtcctttgcaactgacttcgcggatatggttcatttcagtactgacaatgagtaatatcagggctatgatggagcgggtagctcagtcgattacagtgtgtttaaaccgttcagttcccaagactacttccacccgttctgtttcattcaaaactatgaagatcagactcaggttgaggattgctggctaggagataacactgtctccttgcctgatctcgataccaccaaggatgtggtcaagaatgaatggtacgactgggtgggatcattggtatcgaactactccagtaagatatttctccctcattctacaacttggctgatcgatgatacttacgaaatcagttgacggcctccgtatcgacacagtaaaacacgtccagaaggacttctggcccgggtacaacaaagccgcaggcgtgtactgtatcggcgaggtgctcgacggtgatccggcctacacttgtccctaccagaacgtcatggacggcgtactgaactatcccatgtatggttcctccaaccatgagccttcttgcaagtctcatctcctaacgaaacggctaaaaccagttactatccactcctcaacgccttcaagtcaacctccggcagcatggacgacctctacaacatgatcaacaccgtcaaatccgactgtccagactcaacactcctgggcacattcgtcgagaaccacgacaacccacggttcgcttcgtaagtcttcccttttattttccgttcccaatttccacacagaaccccacctaacaagagcaaagttacaccaacgacatagccctcgccaagaacgtcgcagcattcatcatcctcaacgacggaatccccatcatctacgccggccaagaacagcactacgccggcggaaacgaccccgcgaaccgcgaagcaacctggctctcgggctacccgaccgacagcgagctgtacaagttaattgcctccgcgaacgcaatccggaactatgccattagcaaagatacaggattcgtgacctacaaggtaagcacaacctctaagcataccctaatggcctatcttcagagtatctgacacaagagactaatcactggcaatacagaactggcccatctacaaagacgacacaacgatcgccatgcgcaagggcacagatgggtcgcagatcgtgactatcttgtccaacaagggtgcttcgggtgattcgtataccctctccttgagtggtgcgggttacacagccggccagcaattgacggaggtcattggctgcacgaccgtgacggttggttcggatggaaatgtgcctgttcctatggcaggtgggctacctagggtattgtatccgactgagaagttggcaggtagcaagatctgtagtagctcgtgaagggtggagagtatatgatggtactgctattcaatctggcattggacagtgagtttgagtttgatgtacataaccaaggttgtgtctgtataatatatacatgtaagatacatgagcttcggtgatataatacagaagtaccatacagtaccgcgttatgaaaacacattaatccggatcctttcctataatagactagcgtgcttggcattagggttcgaaaaacaatcgaagagtataaggggatgacagcagtaacgactccaactgtagcccacatcttgagttcggcaactactgttggcacgtgaccctgtgccttgtggtagctccttaactttgtcatcattcgaagaattttcgtcccttcccaggtaccatccaaaagacaagcatccgtcgcttcactctgagatcagatgagagtaatattgttgactgcgtttgtgatgcgggtgatgtcctctgcgatcggccgcaagctgtttagtttgccccggatcttctgtgccgacggttgctccccgaattttcttagctagtgtaatcacgctattcagaaaggcttccaagaattaggccggtagttcggcgcgtttggtgtcgtcaagctccagcagtgctggggcctcggctatgatatggttagaatgctcggggtgggtcacggcaggacacccgacactgcaacgtctaccacatttgagcgttattggcagacttgcggcgagataacgaccgctagcttgtatcaaccaaatccaactgaaattattgctttgccatcccaacagtggatttcggaggagggaggggggaagatatacgatgaacggaagactggacaagatacgttacataaagcagtactacttgtttcaaactgtgtacacaccagggctctcgcttcagcggagagtgtcgaaagattcagtaaaacatcgccaggggtgatggaaaggggttaagctagacacagaaacatagaggaatcaagaatgagagaagacgttgtgaagctttgttcgacgtatttcgcagagcatatttctgagcagcggacacgatttgtaacgtagccgtagactcttgggactgaagcttcacgaagggcagaagaaagtgaagtgcagcgtctgaatcgatattctgcctatacagccgatagttttcccctgaatctatcaaatggccaagtgttcgcagcacttctgggcgccttccgcttaaacgtatgccctgaaggagcccagtgaacgagtaaaaatcgcgcaggcgataaaatttctgcggtcggtttagtatgaaccaaggcaagggaaggagataattaccagcgccaattgatccaactttagatacaaagccggttcagtagctgagcattcctctgctgctcggcaaatactgttccaccacctattcagagctgtcaaagggtcgccgctacccttcttcaccatttcgacggtgagctcctgaaagagggaaagagctgctgccgtaagctctgctgccagtgcctccagttcctccagctccgtgtggtagagtttgtcaagaaatgcagtttgagtattgaagtcttgcgaacagacaacttctggacttctgtagaaatttcggaagcgtacggccagagcttcaagttggcaatggataagggcgatcgggttgtcttttgagaggactgcttttatcggatccgtgataaatcgagcatcaatgttgtattccgtgtatcgccgattacaaacgcattcacacgcatgatacaccgctccggacagaaaggggtctgcggtaaatttctctaaagtctttgaggcgtcaggatatgcagtggatggataggaagcggaagggtgatacatttgtcaagcctagatacttctcaaactcgttcaagtgccttctgaggtagtaatacagaatcacgcttagcccatcatacttagactcaagcgccttgacaataggtgatgagcgatcccttgcttcttgcacccagtcctcgaattgaacaagaggaaagtacctttcactctcgtcaatcagttgctgcgcaattgtgttggcatcggaatcccatgacgaacgcggttgccacttccaacgaatggagtccagtgcttctggtatcttgggtacgtttgtaccggatgactcaaagcggaatccaatcaactgcgaatgaaattgtggcggctggctagatggtttttcgcccgtaagcaaaacaggttcgagttctacatcataggcagttgtggcaacgctcaacgaagatcgctcgcaaggtaatagaaagatcatggtgaaaagaaatctagcagaagattcgaactgaggtagtagacgagcattctagcaggaattgtggtacgtttatatggaatacttgatgccggcgccgcaataagtagcaaagggattgcagaaagttctataggggacaacacagtaaaaaggcggagattgcagaaaaatacagggagacagcagatttgaagatccaggccttgatctggactgggaggacaccgacctcggcagtggctgcatcttactggtggatatggttgggtgttcaaacaggtcacaccctctatcctatcaggcgagaggctacccatagtgcactgtccttcctgctttacggcacatccagcacccgattgaaataggggccgtcgaggtgtcctctcctccgacctgcgtcaaggaaacctactcctttttctgccctcgtcaagctgttcacttttccttgaaaatggtcaaacaaactcgacttcctcttctacttgcagaagcattgccttgtcgcctagaccgtattcaggccaaatttacggagcaatgcaaggatctgaaaccggacgcctttctccagcttacagttcttctcagccagattgaacacatagttggaattcattacacaccaggagtttcaacatcagctagcacaaaacctacttgcgtcgtctgcggccgatcatactcaagaatatcttccctgaactctcatatctcgctagcccatcagtatctgcggcggatcattgaagccagatcttgcaattcttgcgacaatgaattcgactccccaaggcaacttgtctaccatgagagatcgattcacaaggcagcgtatctgtccagagcagactttatctggccaggatttgaacaactgaactcaagagaaggtgggagtgaccgtgcattgacaagactggcgaatagatgctaatttatcgttccagcgcagaagtctttcctgagaaccctggcaggcgatgaggaagagccaaaagtttatgaatttgtgggggaagcagctgacacaaaacggacaactttagagccgggggaagcggtagaagggagacgttttgatgagcaatattctatcagcggtgacggacatttgcaatacgatttagggtattatggggatatagactcctggttactaccgtattcaccggcttgtaacggatccgtctgatgtctccatgtttcttgagcggaagtactatacatccctagtcaatcaaacggtcgttgttgcaaatatactatctcggccaaaattccggcctgtccttgaatgtaaggtattctccagtccttcatccatcccgcaacacagatgctgttttccgccatcgttagagacttcgtgagtagaatgtcagaatgacttacatatcggttccgcttagcaaaacgcttttccgtaagtgttcgtttggagtgaaatattcgaatatccaagtagaattgctttgaccaccgaggtgaaacacgttgatgagtttaagatcgggcaaaccgctggatggcacgaagaggctgttgcttttattcatttcgcgctttcgaaaacggcccggaagatatgtgccttcgtccacccggagaaactgcttaacggcctgaacaattagcccagaagctctataaactcttggtattctatacagacatcattatgctcgatttgcctctgctctattgtggcatcgaggaaagtttgattttgcatcacaatccagccgtccaccatgaactttcttcgcgtatcgataacttgtcccttgtcgcggacagaaacagaatcaactgagccatcgcgaaaaaggaagctgacgactgatgaactcatattgctcgctggttgctgtatcgttgaccccgccagtcgtgtgcggggcagattcatgaatgtcggaagggcggacagcacgtgagagtgggcgaagagggacactggagcgactttctcgagtcaggaaaggaacatccagttcttcgtagtggagcactcaccacttccagcttggcagctaatccatatctcattattatccgccataatgacaataaaattttgtctttgattggtttattcactctaggacgccaaagatgaacaatatatgagtggaaaggatgggaaatggaaggtagtcgtcgggtggaagcaatgtgaagactttgggaagttcaacggtcaggttttctccctcaatctcaattctgcggataaagcgagagaagtaattgcagtatgttttttctaagcgactacagataatactttgagcaaaggcctactataaatcgttacgtcgcaaaaatactgaatacgtttgccgattacgaagaggacagggcaggattaggcaacagcgcagaaagtacaagagagattgcagtattaaccaggcagaaaacggataatattctgagcaaaggcttaccataaatcggcacgtcgcagaaatactggatacgtttgcagattacgaagaggataaggcaggtgcagaaggttcaagagagattgcagttattaatcaagtggttatcctataatcaagttatctcattggatgagccgtacgttaccattaactagtgttcatgctaaatacatagttggctcacggccaagtaggtggttcatcgcatgccttctttggcagcaaaattagtctactcccactcccgtccctacttagtatcatagcaccacccttcaaggagaggaagtgtacattatcccgtaccacttactcaaattgtagggggacgctaccgcgacaccggccaggctgcctccacaccagcctccgcttacgccacatccttccgcctaccttaataggtaaggctcgctccctataaggtaaggcttgcttttctgagccagcacaataataccgcctacccgttttgaggcagtagtttattatctcaggcagtacaactggtgtctcaagcaagaataactctatattaggaaagcagtaatattacactggctataagaagtgggccttatcttatagggagtgaaccttacctaccaaggtaggcagaagcatgtggcatgagcggaggctggtgtcgcggtagcgcctcccaagttataaactcatctgtgtgatgcaatgcggaacaactctactagtacatgtttgccttagaaacaaagtaacaactgcaaccagcccgcaaccttccgccataatatcagtcttctaatcttccgacatgttacattaatgcccatgcgatacgta

SEQ ID NO: 70 - protein, 499 aa, artificial sequence; identical to SEQ ID NO: 16
  Synthetic Construct

SEQ ID NO: 71 - DNA, 1515 bp, artificial sequence
  The nucleotide sequence of A.niger fcy1 in pHUda1078 & 1067
ttctgtggttcttgccatgatcgtggatcagaggttcagagaacaatctctgcgataagaagatatactagtaaatagtgcgtggcatggggtctgcgcgagatgaaccccgagtttatcagccactgccagtttgatcttgtaaattgtgaaactgtgaattaatggttacaagtgataaggacgttaccatcgggctctatgcatctagatcggatgtctcatatacaatcagctcaatttgtattcagttatagttgtatacaaggcatgaaatattaagcatctttcttacgcttatgcatgtcgatccccaagcacaccaaagaagcactttatgcataccataacccaagaaagtctatcacatgcacacattatccatgaaaatactattcaatacgaatgtaacaacgtcctctatcagtctcaatgacagcagctatcttgttatcatggagctccgcacgtccagcccgatgcgggtcagtccggcagttaaccacacagagtttgctccgtcttgatgctaccccatctttctatctctctcccaattacccctccaatcgctctatatttcatatctcaatacagcatacaacaagcacataccatcatggagaccgatcccggattcatcgctgctgtggaagaagccaagcaaggcgctgctgagggtggtgtgcccattggagcttgtttggtctccaaggatggcaagattctaggccgcggccacaatatgcgcgtccagaagggtagtcccgtgttgcatgttcgttgatcccatcccttgccttctgagggtcgtctggggttctaattctaatctctaccgtcataggctgagatgtccgcgctcgagaactccggtcgtctgcccgcttcggcctacgaaggcgctactatgtacacgaccctgtcgccatgcgacatgtgcaccggtgcctgcatcctctacaaggttaagcgcgttgttgtgggcgagaacaagagcttcatgggtggcgaggactatcttaagagccgtgggaaggaggttgtggttttggataatgcagagtgtaagcagctgatggagaagttcatgaaggagaagccggagctttggtaggtttcccatgcatctcactggactggtctagtcttttgttggaatgtacgctgactgtacgatgtctttgcaggaatgaggacatttccgtctgagcttttgaattcgtgaaggtgtcaactatattgctggctaggctctcatgtacataataaagaattgaaagctagttctggtcgcattgagcacccaatttagaccgtcagacggtggatctcttcgaagaagaacttgagatcatccgggttgacgaaaggagtcacacctgtgatacattagcatttattgaataacccagctgtggcagtgctcaccgtgacccaagttggcaatccagccttgcttgcccttctcgaatccccgaaccatagtctccacagcctccgtgatagcctcgc

SEQ ID NO: 72 - protein, 168 aa, artificial sequence; identical to SEQ ID NO: 60
  Synthetic Construct

SEQ ID NO: 73 - DNA, 24 bp, artificial sequence
  Forward primer
tcgagtgcggccgacgcgtacgtc

SEQ ID NO: 74 - DNA, 20 bp, artificial sequence
  Reverse primer
cagagagtgttggtcacgta

SEQ ID NO: 75 - DNA, 52 bp, artificial sequence
  Primer bac-F
tctagagaataggaactcggaataggaacttcaagatgaattcgcggccgcg

SEQ ID NO: 76 - DNA, 77 bp, artificial sequence
  Primer bac-R
tctagattgaagttcctattccgagttcctattcttcaaatagtataggaacttcagcatgcaagcttggcctccgc

SEQ ID NO: 77 - DNA, 30 bp, artificial sequence
  Primer FLP-F
ttaattaatggaagtgcgttgatcattatt

SEQ ID NO: 78 - DNA, 30 bp, artificial sequence
  Primer FLP-R
ttaattaaactagtggagcgaaccaagtga

SEQ ID NO: 79 - DNA, 24 bp, artificial sequence
  Forward primer
tcgagtgcggccgacgcgtacgtc

SEQ ID NO: 80 - DNA, 20 bp, artificial sequence
  Reverse primer
cagagagtgttggtcacgta

SEQ ID NO: 81 - DNA, 27 bp, artificial sequence
  Primer 126-F
ggatccaccatgcggctctccacatcc

SEQ ID NO: 82 - DNA, 30 bp, artificial sequence
  Primer 126-R
cacgtgtgattacggacacaatccgttatt

SEQ ID NO: 83 - DNA, 1815 bp, artificial sequence
  The nucleotide sequence of the JA126 amylase gene
atgcggctctccacatcctccctcttcttgtccgtctccttgctcggaaagttggccttgggcgcgacgtcggacgattggaagggtaaggccatttaccagttgctcacggaccgattcggtcgcgcagatgactcgacctcgaactgttcgaacctctcgaactactgtggtggcacttacgagggcatcactaaacatctcgactacatctccggtatgggcttcgatgcaatttggatttcgccgatccctaagaactcggacggtggataccacggttactgggccacagacttctatcagctcaactcgaacttcggcgacgagtcgcagttgaaagcgctcatccaggcggcccatgagcgggacatgtatgtcatgctcgatgtggtggcaaaccacgccggcccgacttcgaacggatactcgggttacactttcggtgatgcctccctctaccatccgaaatgtaccatcgattacaacgatcagacatcgatcgaacagtgttgggtcgccgatgagttgcccgatatcgacaccgaaaactcggacaacgtcgcaatcctcaacgacatcgtctccggctgggtgggtaactactcgttcgatggtattcggatcgacaccgtcaagcacatccgcaaggacttctggacaggttacgccgaagccgcgggtgtgttcgcgaccggagaggtgttcaacggagaccccgcatacgtgggaccctatcagaaatacttgccttccctcatcaactatcccatgtactacgccctcaacgacgtcttcgtctcgaagtcgaagggtttctccaggatttccgagatgttgggctcgaaccgtaacgccttcgaagatacttccgtcctcaccacgttcgtggacaaccacgacaaccctcgattcttgaactcccagtccgacaaagccctcttcaagaacgcgctcacatacgtgttgctcggcgaaggaatccccatcgtctactatggatcggaacagggcttctcgggcggtgcagaccctgccaaccgagaagtcctctggactacgaactacgacacgtcgtcggatctctaccagttcatcaagaccgtcaactcggtgcgtatgaagtcgaacaaggcggtgtacatggacatttacgtgggcgataacgcgtatgcattcaagcatggagacgccttggtggtcctcaacaactacggctcgggttcgaccaaccaggtgtccttctcggtgtcgggaaagttcgactccggcgcctccctcatggatatcgtgtccaacatcacaactactgtctcctcggatggcacagtcactttcaacttgaaggatggcctcccggcgattttcacctccgcaactggcggcaccactacgacggctacccccactggctccggcagcgtgacctcgaccagcaagaccaccgcgactgccagcaagaccagcaccagtacgtcatcaacctcctgtaccactcccaccgccgtggctgtgactttcgatctgacagctaccaccacctacggcgagaacatctacctggtcggatcgatctctcagctgggtgactgggaaaccagcgacggcatagctctgagtgctgacaagtacacttccagcgacccgctctggtatgtcactgtgactctgccggctggtgagtcgtttgagtacaagtttatccgcattgagagcgatgactccgtggagtgggagagtgatcccaaccgagaatacaccgttcctcaggcgtgcggaacgtcgaccgcgacggtgactgacacctggcggtag

SEQ ID NO: 84 - protein, 604 aa, Artificial sequence
  The amino acid sequence of the JA126 amylase
MRLSTSSLFLSVSLLGKLALGATSDDWKSKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 85 - DNA, 29 bp, artificial sequence
  Forward primer
tcgaacttcggcgacgagtcgcagttgaa

SEQ ID NO: 86 - DNA, 30 bp, artificial sequence
  Reverse primer
cccaacatctcggaaatcctggagaaaccc

SEQ ID NO: 87 - DNA, 27 bp, artificial sequence
  Primer 172449
gacgaattccgatgaatgtgtgtcctg

SEQ ID NO: 88 - DNA, 60 bp, artificial sequence
  Primer 172450
gacgaattctctagaagatctctcgaggagctcaagcttctgtacagtgaccggtgactc

SEQ ID NO: 89 - DNA, 27 bp, artificial sequence
  Primer X4407C0
cagggatccgtctaggctgcaataggc

SEQ ID NO: 90 - DNA, 18 bp, artificial sequence
  Primer X4407C07
ggagaattcggtcacatc

SEQ ID NO: 91 - DNA, 26 bp, artificial sequence
  Primer X7164D09
gacactagtcgtcggcagcaccggtg

SEQ ID NO: 92 - DNA, 28 bp, artificial sequence
  Primer X7164D10
cagaagcttcagagtgaaatagacgcgg

SEQ ID NO: 93 - DNA, 79 bp, artificial sequence
  Primer T5483H12
gcacatatgatttaaatccctaatgttgaccctaatgttgaccctaatgttgagcggccgcgtttaaacgaattcgccc

SEQ ID NO: 94 - DNA, 71 bp, artificial sequence
  Primer T5483G10
cgtaagcttatttaaatccctaatgttgaccctaatgttgaccctaatgttgagaccggtgactctttctg

SEQ ID NO: 95 - DNA, 27 bp, artificial sequence
  Primer D5831F08
gacgaattcggcgtgggaaattcctgg

SEQ ID NO: 96 - DNA, 18 bp, artificial sequence
  Primer D5831F09
ccctacacctggggtacc

SEQ ID NO: 97 - DNA, 29 bp, artificial sequence
  Primer D5775F04
gacgcggccgcgctttgctaaaactttgg

SEQ ID NO: 98 - DNA, 30 bp, artificial sequence
  Primer D5775D07
gacaagcttatgctcgatggaaacgtgcac

SEQ ID NO: 99 - DNA, 30 bp, artificial sequence
  Primer D5775D08
gacaagcttacagtagttggactactttac

SEQ ID NO: 100 - DNA, 28 bp, artificial sequence
  Primer D5775F05
gacgcggccgcgacgagcaactgacggc

SEQ ID NO: 101 - DNA, 60 bp, artificial sequence
  Primer F3-1
gatccttgaagttcctattccgagttcctattcttcaaatagtataggaacttcactgca

SEQ ID NO: 102 - DNA, 50 bp, artificial sequence
  Primer F3-2
tgaagttcctatactatttgaagaataggaactcggaataggaacttcaa

SEQ ID NO: 103 - DNA, 55 bp, artificial sequence
  Primer F-1
gtaccttgaagttcctattccgagttcctattctctagaaagtataggaacttca

SEQ ID NO: 104 - DNA, 54 bp, artificial sequence
  Primer F-2
gtactgaagttcctatactttctagagaataggaagtcggaataggaacttcaa

SEQ ID NO: 105 - DNA, 2091 bp, Artificial sequence
  A Talaromyces emersonii AMG gene containing introns optimized for expression in Aspergillus.
ggatccaccatggcctcgctcgtcgcaggagccctctgtatcctcggcttgacacctgcagccttcgcacgagcacccgtcgcagcacgggcaaccggttcgttggattccttcctcgcaaccgaaactcctatcgccctccagggcgtgctcaacaacatcggacccaacggtgcggacgtcgcaggagcgtccgcaggcattgtcgtcgcctcgccctccaggtccgatcccaactgtaggttctttcccaccagaaattacttatttaaatcagccctctgacaggttgaagacttctattcgtggacgagggatgcagcgttgacagcgaaatacctcgtcgatgccttcattgccggaaacaaagacttggagcagacaatccagcagtacatctcggcacaggcgaaggtgcagaccatctcgaacccctccggtgacttgtcgacaggcggattgggcgaacccaaattcaacgtcaacgagaccgccttcacaggaccctggggtcgaccccagagggacggacctgccctcagggcaaccgcactcatcgcgtacgccaactacttgattgtaagcttctgctcgctgcccttctctctgctcgtatgctaagtagtcctgtcaggataacggagaggcgtccacagccgatgagatcatctggcctatcgtccagaacgacctctcctacatcacccagtactggaactcctccacgttcggtaggcaaatgaatattcccgacacagcgtggtactaatttgattcagatttgtgggaggaggtcgaaggctcgtccttcttcactacagccgtgcagcatcgagccttggtggaaggtaacgcgttggcgacgcgattgaaccacacatgttccaactgtgtgtcccaggcaccgcaggtcctctgtttcctccagtcctactggactggatcgtacgtcttggcgaacttcggtggctccggcaggtccggcaaggacgtgaactccatcctcggctccatccatacattcgatcctgccggaggatgtgatgactcgaccttccagccctgttccgcaagggccttggcaaaccataaggtcgtcaccgattcgttccgctcgatctacgcgatcaactccggcatcgccgaaggttcggcagtggcagtgggtcgataccccgaagacgtctatcagggtggcaacccctggtatctcgcaacagccgcagcggcagagcagctctacgacgcaatctatcagtggaagaagattggttcgatttccattaccgacgtgtccctcccgttcttccaggatatctacccgtcggcagccgtcggaacctataactcgggctccacaaccttcaacgacatcatttcggcagtccagacgtatggagatggctatttgtcgatcgtggtacgttttgccttagattctcaggtgtaaagaaaaaaatggaactaactcagttctaggaaaagtacacaccctccgatggatcgctcacggagcagttctcgcgcacggatggaacccccttgtccgcgtcggcattgacgtggtcgtatgcctcgttgttgactgcctcggcacgacggcagtccgtcgtccctgcctcgtggggagagtcgtcggcgtcgtcggtccctgcagtctgttccgcaacttcggccactggcccttattccactgcaaccaacactgtctggccttcgtcgggctccggatcgtcgacaaccacgtcgtcggcaccttgtaccacgcctacatccgtcgccgtcaccttcgacgagatcgtgtcgacctcgtacggtgaaactatctacctcgcaggatcgatccccgagctcggcaactggtcgaccgcgtccgccatccccctccgagccgacgcatacacaaactccaaccccttgtggtatgtcacggtgaacttgcctcctggcacctccttcgagtacaagttcttcaaaaaccagaccgatggtaccatcgtctgggaggacgaccccaaccgttcgtataccgtccctgcgtactgtggtcagactaccgccattctcgatgactcctggcagtgactcgag

SEQ ID NO: 106 - DNA, 20 bp, artificial sequence
  Primer oJaL132
cagatactggttccttacgg

SEQ ID NO: 107 - DNA, 40 bp, artificial sequence
  Primer oJaL133
cgtccacgcggggattatgcgtagaatgcagagatagctg

SEQ ID NO: 108 - DNA, 20 bp, artificial sequence
  Primer X1111C07
gcataatccccgcgtggacg

SEQ ID NO: 109 - DNA, 19 bp, artificial sequence
  Primer oJaL114
ccaacagccgactcaggag

SEQ ID NO: 110 - DNA, 40 bp, artificial sequence
  Primer oJaL134
cgataagctccttgacggggttgagcactgcttttggatc

SEQ ID NO: 111 - DNA, 20 bp, artificial sequence
  Primer oJaL135
gctcacccggcataagttgc

SEQ ID NO: 112 - DNA, 20 bp, artificial sequence
  Primer X1111C08
ccccgtcaaggagcttatcg

SEQ ID NO: 113 - DNA, 19 bp, artificial sequence
  Primer oJaL113
gagctgctggatttggctg

SEQ ID NO: 114 - DNA, 29 bp, artificial sequence
  Primer K6763E12
gacgcggccgccgcgtggaggtctaggac

SEQ ID NO: 115 - DNA, 27 bp, artificial sequence
  Primer K6763F01
gacaagcttacaaacccgtgacactcc

SEQ ID NO: 116 - DNA, 28 bp, artificial sequence
  Primer K6763F02
gacaagcttacgcatgtatgtatgtgtc

SEQ ID NO: 117 - DNA, 28 bp, artificial sequence
  Primer K6763F03
gacgtttaaacggatgggtttgccatac

(19) United States
(12) Patent Application Publication
(10) Pub. No.: US 2014/0113304 A1
(43) Pub. Date: Apr. 24, 2014

(54) Bi-Directional Cytosine Deaminase-Encoding Selection Marker

(71) Applicant: Novozymes A/S, Bagsvaerd (DK)

(72) Inventor: Hiroaki Udagawa, Ichikawa (JP)

(73) Assignee: Novozymes A/S, Bagsvaerd (DK)

(21) Appl. No.: 14/119,000

(22) PCT Filed: May 23, 2012
(86) PCT No.: PCT/EP2012/059604

§ 371 Date: Jan. 7, 2014

(30) Foreign Application Priority Data
May 23, 2011 (EP) 11167049.3

(51) Int. Cl. C12N 15/52; C12N 15/80; C12N 9/78

(52) U.S. Cl. CPC C12N 15/52; C12N 9/78; C12N 15/80
USPC 435/6.18; 536/23.2; 435/320.1; 435/444; 435/445; 435/442; 435/448; 435/471; 435/254.11; 435/254.3; 435/254.7; 435/254.8; 435/254.4; 435/254.5; 435/254.6

(57) ABSTRACT

The present invention relates to a bi-directional cytosine deaminase-encoding selection marker as well as methods for using said marker.

### REFERENCE TO SEQUENCE LISTING

This application contains a Sequence Listing in computer readable form. The computer readable form is incorporated herein by reference.

### FIELD OF THE INVENTION

The present invention relates to a bi-directional cytosine deaminase-encoding selection marker as well as methods for using said marker.

### BACKGROUND OF THE INVENTION

There is a constant need for new tools in molecular biology, even simple tools, such as novel selection markers are in demand, especially bi-directional markers suitable for use in filamentous fungal host cells.

Cytosine deaminase (EC 3.5.4.1) catalyzes the deamination of cytosine and 5-fluorocytosine (5FC) to form uracil and toxic 5-fluorouracil (5FU), respectively. When genetically modified cells comprising cytosine deaminase are combined with 5FC it is converted to toxic 5FU, so the cytosine deaminase-encoding gene is potentially a potent negative selection marker.

It has also been shown that inhibitors in the pyrimidine de novo synthesis pathway can be utilized to create a condition in which cells are dependent on the conversion of pyrimidine supplements to uracil by cytosine deaminase. Thus, only cells expressing the cytosine deaminase gene can be rescued in a positive selection medium (Wei and Huber, 1996, J Biol Chem 271(7): 3812).

### SUMMARY OF THE INVENTION

In a first aspect, the invention provides an isolated polynucleotide encoding a polypeptide having cytosine deaminase activity, said polypeptide selected from the group consisting of:

(a) a polypeptide having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO:60;

(b) a polypeptide encoded by a polynucleotide that hybridizes under medium stringency conditions, medium-high stringency conditions, high stringency conditions, or very high stringency conditions with (i) the polypeptide coding sequence of SEQ ID NO:59, (ii) the cDNA sequence thereof, or (iii) the full-length complement of (i) or (ii);

(c) a polypeptide encoded by a polynucleotide having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide coding sequence of SEQ ID NO:59 or the cDNA sequence thereof;

(d) a variant of the polypeptide of SEQ ID NO:60 comprising a substitution, deletion, and/or insertion at one or more positions; and

(e) a fragment of the polypeptide of (a), (b), (c) or (d) that has cytosine deaminase activity.

In a second aspect, the invention relates to a nucleic acid construct or expression vector comprising the polynucleotide of the first aspect operably linked to one or more control sequences that directs the production of the cytosine deaminase polypeptide in an expression host cell.

A third aspect of the invention is method of using the polynucleotide of the first aspect as a negative selection marker, comprising the steps of:

(a) providing a host cell comprising one or more cytosine deaminase-encoding polynucleotide of the first aspect;

(b) transforming the host cell with an integrative nucleic acid construct which, when site-specifically integrated in the host genome, inactivates at least one cytosine deaminase-encoding polynucleotide, so the resulting host cell produces less or no cytosine deaminase compared with the host cell of step (a);

(c) cultivating the transformed host cell in a selective medium comprising a sufficient amount 5-fluorocytosin, which is converted to an inhibitory concentration of toxic 5-fluorouracil by cytosine deaminase; and

(d) selecting a resulting host cell with reduced or no measurable cytosine deaminase activity which can grow in the selective medium.

In a fourth aspect, the invention relates to a method of using the polynucleotide of the first aspect, or the nucleic acid construct or expression vector of the second aspect, as a positive selection marker, comprising the steps of:

(a) providing a host cell without measurable cytosine deaminase activity;

(b) transforming a host cell with a nucleic acid construct comprising at least one expressible cytosine deaminase-encoding polynucleotide of the first aspect,

(c) cultivating the transformed host cell in a medium comprising a de novo pyrimidine synthesis inhibitor as well as inosine and cytosine under conditions conducive for the expression of the cytosine deaminase; and

(d) selecting a growing host cell, which comprises at least one cytosine deaminase-encoding polynucleotide.

One aspect of the invention relates to a a method of producing a mutant of a parent host cell, comprising inactivating a polynucleotide of the first aspect, which results in the mutant producing less of the encoded cytosine deaminase polypeptide than the parent cell.

Another aspect of the invention relates to a recombinant host cell comprising at least one chromosomally integrated polynucleotide according to the first aspect operably linked to one or more control sequences that direct the production of the encoded cytosine deaminase.

A final aspect of the invention relates to the use of a polynucleotide as defined in the first aspect or a nucleic acid construct or vector as defined in the second aspect as a selection marker in a microbiological transformation process, where a polynucleotide of interest is transformed into a suitable microbial host cell which is then cultivated under conditions of positive or negative selection pressure to select for the presence or absence of the selection marker.

### BRIEF DESCRIPTION OF DRAWINGS

FIG. 1 shows the basic scheme of the FRT/FLP system in the experiments exemplified with pyrG as bi-directional selective marker.

FIG. 2 shows a plasmid map of pHUda981 (Pgpd, HSV1 tk, TtrpC are described in WO07045248).

FIG. 3 shows a plasmid map of pHUda1019.

FIG. 4 shows a plasmid map of pHUda1000.

FIG. 5 shows the schematic NA1 (upper panel) and acid stable amylase loci (lower panel) after the pHUda1000 was introduced correctly in NN059183.

FIG. 6 shows a plasmid map of pHUda801.

FIG. 7 shows a plasmid map of pHUda1043.

FIG. 8 shows a plasmid map of pHUda1078.

FIG. 9 shows a plasmid map of pHUda1067.

FIG. 10 shows the schematic NA1 locus (upper), NA2 locus (middle) and acid stable amylase locus (lower) in NN059208.

FIG. 11 shows a plasmid map of pRika147.

FIG. 12 shows the schematic NA1 (upper), NA2 (middle) and acid stable amylase loci (lower) after the correct integrations of pRika147 in NN059208.

### DEFINITIONS

Cytosine deaminase: Cytosine deaminase (EC 3.5.4.1) catalyzes the deamination of cytosine and 5-fluorocytosine (5FC) to form uracil and toxic 5-fluorouracil (5FU), respectively. When genetically modified cells comprising cytosine deaminase are combined with 5FC it is converted to toxic 5FU, so the cytosine deaminase-encoding gene is potentially a potent negative selection marker.

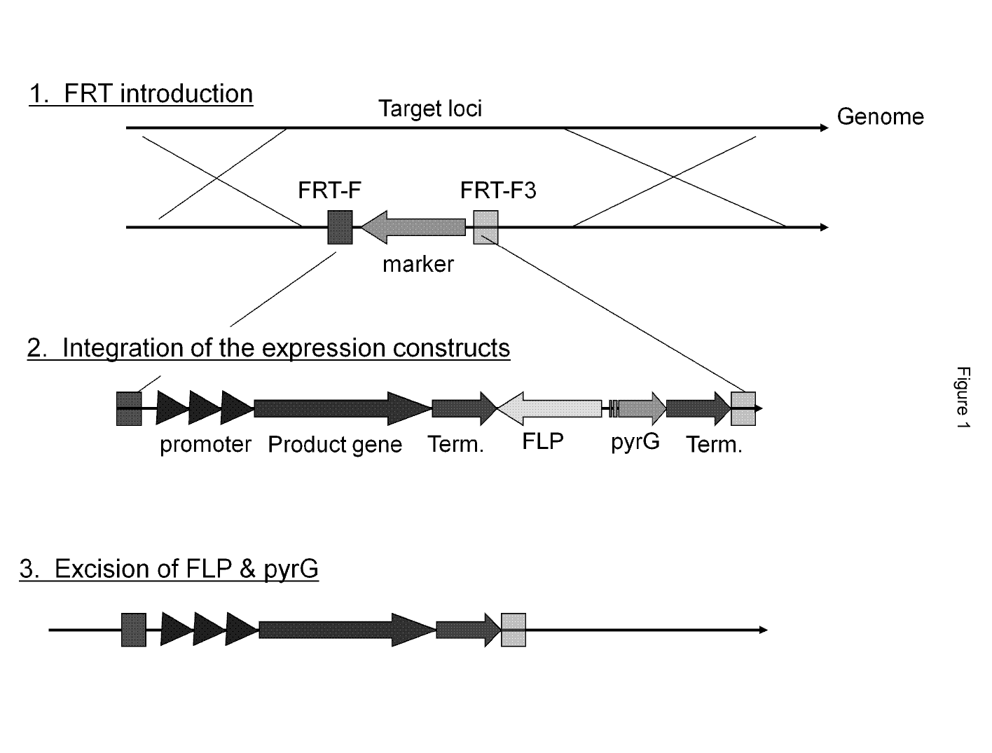

It has also been shown that an inhibitor in the pyrimidine de novo synthesis pathway can be utilized to create a condition in which cells are dependent on the conversion of pyrimidine supplements to uracil by cytosine deaminase. Thus, only cells expressing the cytosine deaminase gene can be rescued in a positive selection medium comprising an inhibitor of the pyrimidine de novo synthesis as well as inosine and cytosine (See FIG. 1 of Wei and Huber, 1996, J Biol Chem 271(7): 3812). The inhibitor is preferably N-(phosphonacetyl)-L-aspartate (PALA), which inhibits aspartate carbamyl transferase.

If necessary, cytosine deaminase activity may be quantitated by a genetic assay (Frederico L. A. et al, 1990, Biochemistry 29: 2532-2537).

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide or a catalytic domain having one or more (e.g., several) amino acids deleted from the amino and/or carboxyl terminus of a mature polypeptide or domain; wherein the fragment has cytosine deaminase activity.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated or purified: The term “isolated” or “purified” means a polypeptide or polynucleotide that is removed from at least one component with which it is naturally associated. For example, a polypeptide may be at least 1% pure, e.g., at least 5% pure, at least 10% pure, at least 20% pure, at least 40% pure, at least 60% pure, at least 80% pure, at least 90% pure, or at least 95% pure, as determined by SDS-PAGE, and a polynucleotide may be at least 1% pure, e.g., at least 5% pure, at least 10% pure, at least 20% pure, at least 40% pure, at least 60% pure, at least 80% pure, at least 90% pure, or at least 95% pure, as determined by agarose electrophoresis.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs the expression of the coding sequence.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides deleted from the 5′ and/or 3′ end of a mature polypeptide coding sequence; wherein the subsequence encodes a fragment having cytosine deaminase activity.

Variant: The term “variant” means a polypeptide having cytosine deaminase activity comprising an alteration, i.e., a substitution, insertion, and/or deletion of one or more (e.g., several) amino acid residues at one or more positions. A substitution means a replacement of the amino acid occupying a position with a different amino acid; a deletion means removal of the amino acid occupying a position; and an insertion means adding an amino acid adjacent to the amino acid occupying a position.

### DETAILED DESCRIPTION OF THE INVENTION

The first aspect of the invention relates to an isolated polynucleotide encoding a polypeptide having cytosine deaminase activity, said polypeptide selected from the group consisting of:

(a) a polypeptide having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO:60;

(b) a polypeptide encoded by a polynucleotide that hybridizes under medium stringency conditions, medium-high stringency conditions, high stringency conditions, or very high stringency conditions with (i) the polypeptide coding sequence of SEQ ID NO:59, (ii) the cDNA sequence thereof, or (iii) the full-length complement of (i) or (ii);

(c) a polypeptide encoded by a polynucleotide having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide coding sequence of SEQ ID NO:59 or the cDNA sequence thereof;

(d) a variant of the polypeptide of SEQ ID NO:60 comprising a substitution, deletion, and/or insertion at one or more positions; and

(e) a fragment of the polypeptide of (a), (b), (c) or (d) that has cytosine deaminase activity.

In an embodiment, the present invention relates to isolated polynucleotides encoding a cytosine deaminase polypeptide having a sequence identity to SEQ ID NO:60 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptides differ by no more than ten amino acids, e.g., nine amino acids, eight amino acids, seven amino acids, six amino acids, five amino acids, four amino acids, three amino acids, two amino acids, or one amino acid from SEQ ID NO:60.

The encoded cytosine deaminase polypeptide of the present invention preferably comprises or consists of the amino acid sequence of SEQ ID NO:60 or an allelic variant thereof; or is a fragment thereof having cytosine deaminase activity. In another aspect, the polypeptide comprises or consists of the polypeptide of SEQ ID NO:60.

In another embodiment, the present invention relates to isolated polynucleotides encoding a cytosine deaminase polypeptide that are encoded by a polynucleotide that hybridizes under very low stringency conditions, low stringency conditions, medium stringency conditions, medium-high stringency conditions, high stringency conditions, or very high stringency conditions with (i) the polypeptide coding sequence of SEQ ID NO:59, (ii) the cDNA sequence thereof, or (iii) the full-length complement of (i) or (ii) (Sambrook et al., 1989, Molecular Cloning, A Laboratory Manual, 2d edition, Cold Spring Harbor, N.Y.).

The polynucleotide of SEQ ID NO:59 or a subsequence thereof, as well as the polypeptide of SEQ ID NO:60 or a fragment thereof, may be used to design nucleic acid probes to identify and clone DNA encoding polypeptides having cytosine deaminase activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having cytosine deaminase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO:59 or a subsequence thereof, the carrier material is preferably used in a Southern blot.

For purposes of the present invention, hybridization indicates that the polynucleotide hybridizes to a labeled nucleic acid probe corresponding to (i) SEQ ID NO:59; (ii) the polypeptide coding sequence of SEQ ID NO:59; (iii) the cDNA sequence thereof; (iv) the full-length complement thereof; or (v) a subsequence thereof; under very low to very high stringency conditions. Molecules to which the nucleic acid probe hybridizes under these conditions can be detected using, for example, X-ray film.

For probes of at least 100 nucleotides in length, very low stringency conditions are defined as prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours optimally. The carrier material is finally washed three times each for 15 minutes using 2×SSC, 0.2% SDS at 45° C.

For probes of at least 100 nucleotides in length, low stringency conditions are defined as prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours optimally. The carrier material is finally washed three times each for 15 minutes using 2×SSC, 0.2% SDS at 50° C.

For probes of at least 100 nucleotides in length, medium stringency conditions are defined as prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours optimally. The carrier material is finally washed three times each for 15 minutes using 2×SSC, 0.2% SDS at 55° C.

For probes of at least 100 nucleotides in length, medium-high stringency conditions are defined as prehybridization and hybridization at 42° C. in 5×SSPE, 0.3% SDS, 200 micrograms/ml sheared and denatured salmon sperm DNA, and either 35% formamide, following standard Southern blotting procedures for 12 to 24 hours optimally. The carrier material is finally washed three times each for 15 minutes using 2×SSC, 0.2% SDS at 60° C.

For probes of at least 100 nucleotides in length, high stringency conditions are defined as prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours optimally. The carrier material is finally washed three times each for 15 minutes using 2×SSC, 0.2% SDS at 65° C.

For probes of at least 100 nucleotides in length, very high stringency conditions are defined as prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours optimally. The carrier material is finally washed three times each for 15 minutes using 2×SSC, 0.2% SDS at 70° C.

In another embodiment, the present invention relates to isolated to isolated polynucleotides encoding a cytosine deaminase, said polynucleotides having a sequence identity to the polypeptide coding sequence of SEQ ID NO:59 or the cDNA sequence thereof of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to isolated polynucleotides encoding a variant of the cytosine deaminase polypeptide of SEQ ID NO:60 comprising a substitution, deletion, and/or insertion at one or more (e.g., several) positions. Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for cytosine deaminase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

In an embodiment, the number of amino acid substitutions, deletions and/or insertions introduced into the polypeptide of SEQ ID NO:60 is not more than 10, e.g., 1, 2, 3, 4, 5, 6, 7, 8 or 9. The polypeptide may be a hybrid polypeptide in which a region of one polypeptide is fused at the N-terminus or the C-terminus of a region of another polypeptide.

In a final aspect, the invention relates to a recombinant host cell comprising at least one chromosomally integrated polynucleotide according to the first aspect operably linked to one or more control sequences that direct the production of the encoded cytosine deaminase.

### Sources of Polypeptides Having Cytosine Deaminase Activity

A polynucleotide encoding a polypeptide having cytosine deaminase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted.

The cytosine deaminase polypeptide may be a fungal polypeptide. For example, the polypeptide may be a yeast polypeptide such as a Candida, Kluyveromyces, Pichia, Saccharomyces, Schizosaccharomyces, or Yarrowia polypeptide; or a filamentous fungal polypeptide such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide.

In another aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide.

In another aspect, the polypeptide is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride polypeptide.

It will be understood that for the aforementioned species the invention encompasses both the perfect and imperfect states, and other taxonomic equivalents, e.g., anamorphs, regardless of the species name by which they are known.

Those skilled in the art will readily recognize the identity of appropriate equivalents. Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) using the above-mentioned probes. Techniques for isolating microorganisms from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are well known to those of ordinary skill in the art (see, e.g., Sambrook et al., 1989, supra).

### Nucleic Acid Constructs

In a second aspect, the present invention also relates to nucleic acid constructs or expression vectors comprising a polynucleotide of the first aspect operably linked to one or more control sequences that direct the expression of the cytosine deaminase in a suitable expression host cell.

A polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter sequence, a polynucleotide that is recognized by a host cell for expression of a polynucleotide encoding a polypeptide of the present invention. The promoter sequence contains transcriptional control sequences that mediate the expression of the polypeptide. The promoter may be any polynucleotide that shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus gene encoding a neutral alpha-amylase in which the untranslated leader has been replaced by an untranslated leader from an Aspergillus gene encoding a triose phosphate isomerase; non-limiting examples include modified promoters from an Aspergillus niger gene encoding neutral alpha-amylase in which the untranslated leader has been replaced by an untranslated leader from an Aspergillus nidulans or Aspergillus oryzae gene encoding a triose phosphate isomerase); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, which is recognized by a host cell to terminate transcription. The terminator sequence is operably linked to the 3′-terminus of the polynucleotide encoding the polypeptide. Any terminator that is functional in the host cell of choice may be used in the present invention.

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, when transcribed is a nontranslated region of an mRNA that is important for translation by the host cell. The leader sequence is operably linked to the 5′-terminus of the polynucleotide encoding the polypeptide. Any leader sequence that is functional in the host cell of choice may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell of choice may be used.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Mol. Cellular. Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell of choice may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide.

Where both signal peptide and propeptide sequences are present at the N-terminus of a polypeptide, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked with the regulatory sequence.

In a preferred embodiment of the second aspect, the nucleic acid construct further comprises a polynucleotide encoding a protein of interest, preferably the protein of interest is an enzyme, preferably a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

In the sixth aspect, the present invention also relates to recombinant host cells, comprising at least one chromosomally integrated polynucleotide as defined in the first aspect of the present invention operably linked to one or more control sequences that direct the production of the encoded cytosine deaminase.

Such recombinant host cells are suitable for transformation with an integrative nucleic acid construct comprising a polynucleotide of interest flanked by regions of homology to either the cytosine deaminase encoding gene, or regions up- and downstream of that gene, respectively, in the host cell genome, which direct chromosomal integration by site-specific double homologous recombination, whereby the polynucleotide of interest is integrated into the genome of the host cell while the cytosine deaminase encoding gene is partially or fully excised and thereby inactivated. The successful inactivation of the residing cytosine deaminase encoding gene is selectable in a medium comprising medium comprising 5-fluorocytosin, which is converted to toxic 5-fluorouracil by cytosine deaminase. So, in such a transformation method, the cytosine deaminase encoding gene functions as a negative selection marker, as outlined in the method of the third aspect of the invention.

The fifth aspect of the invention relates to a method of producing a mutant of a parent host cell, comprising inactivating a polynucleotide of the first aspect, which results in the mutant producing less of the encoded cytosine deaminase polypeptide than the parent cell, or even no measurable cytosine deaminase activity whatsoever. A host cell with no measurable cytosine deaminase activity is suitable for a transformation method, where the host cell is transformed with a nucleic acid construct comprising at least one expressible cytosine deaminase-encoding polynucleotide of the first aspect, which is then used as a positive selection marker in a growth medium comprising a de novo pyrimidine synthesis inhibitor under conditions conducive for the expression of the cytosine deaminase, as defined in the fourth aspect of the invention. Preferably, the de novo pyrimidine synthesis inhibitor is N-(phosphonacetyl)-L-aspartate (PALA), which inhibits aspartate carbamyl transferase.

The term “host cell” encompasses any progeny of a parent cell that is not identical to the parent cell due to mutations that occur during replication. The choice of a host cell will to a large extent depend upon the gene encoding the polypeptide and its source.

The host cell may be a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Removal or Reduction of Cytosine Deaminase Activity

In the fifth aspect, the present invention also relates to methods of producing a mutant of a parent cell, which comprises inactivating, disrupting or deleting a polynucleotide of the first aspect, or a portion thereof, encoding a cytosine deaminase, which results in the mutant cell producing less or none of the encoded cytosine deaminase compared with the parent cell, when cultivated under the same conditions.

The mutant cell may be constructed by reducing or eliminating expression of the polynucleotide using methods well known in the art, for example, insertions, disruptions, replacements, or deletions. In a preferred aspect, the polynucleotide is inactivated. The polynucleotide to be modified or inactivated may be, for example, the coding region or a part thereof essential for activity, or a regulatory element required for expression of the coding region. An example of such a regulatory or control sequence may be a promoter sequence or a functional part thereof, i.e., a part that is sufficient for affecting expression of the polynucleotide. Other control sequences for possible modification include, but are not limited to, a leader, polyadenylation sequence, propeptide sequence, signal peptide sequence, transcription terminator, and transcriptional activator.

Modification or inactivation of the polynucleotide may be performed by subjecting the parent cell to mutagenesis and selecting for mutant cells in which expression of the polynucleotide has been reduced or eliminated. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues.

When such agents are used, the mutagenesis is typically performed by incubating the parent cell to be mutagenized in the presence of the mutagenizing agent of choice under suitable conditions, and screening and/or selecting for mutant cells exhibiting reduced or no expression of the gene.

Modification or inactivation of the polynucleotide may be accomplished by insertion, substitution, or deletion of one or more nucleotides in the gene or a regulatory element required for transcription or translation thereof. For example, nucleotides may be inserted or removed so as to result in the introduction of a stop codon, the removal of the start codon, or a change in the open reading frame. Such modification or inactivation may be accomplished by site-directed mutagenesis or PCR generated mutagenesis in accordance with methods known in the art. Although, in principle, the modification may be performed in vivo, i.e., directly on the cell expressing the polynucleotide to be modified, it is preferred that the modification be performed in vitro as exemplified below.

An example of a convenient way to eliminate or reduce expression of a polynucleotide is based on techniques of gene replacement, gene deletion, or gene disruption. For example, in the gene disruption method, a nucleic acid sequence corresponding to the endogenous polynucleotide is mutagenized in vitro to produce a defective nucleic acid sequence that is then transformed into the parent cell to produce a defective gene. By homologous recombination, the defective nucleic acid sequence replaces the endogenous polynucleotide. It may be desirable that the defective polynucleotide also encodes a marker that may be used for selection of transformants in which the polynucleotide has been modified or destroyed. In an aspect, the polynucleotide is disrupted with a selectable marker such as those described herein.

The present invention also relates to methods of inhibiting the expression of a polypeptide having cytosine deaminase activity in a cell, comprising administering to the cell or expressing in the cell a double-stranded RNA (dsRNA) molecule, wherein the dsRNA comprises a subsequence of a polynucleotide of the present invention. In a preferred aspect, the dsRNA is about 15, 16, 17, 18, 19, 20, 21, 22, 23, 24, 25 or more duplex nucleotides in length.

The dsRNA is preferably a small interfering RNA (siRNA) or a micro RNA (miRNA). In a preferred aspect, the dsRNA is small interfering RNA for inhibiting transcription. In another preferred aspect, the dsRNA is micro RNA for inhibiting translation.

The present invention also relates to such double-stranded RNA (dsRNA) molecules, comprising a portion of the polypeptide coding sequence of SEQ ID NO:59 for inhibiting expression of the polypeptide in a cell. While the present invention is not limited by any particular mechanism of action, the dsRNA can enter a cell and cause the degradation of a single-stranded RNA (ssRNA) of similar or identical sequences, including endogenous mRNAs. When a cell is exposed to dsRNA, mRNA from the homologous gene is selectively degraded by a process called RNA interference (RNAi).

The dsRNAs of the present invention can be used in gene-silencing. In one aspect, the invention provides methods to selectively degrade RNA using a dsRNAi of the present invention. The process may be practiced in vitro, ex vivo or in vivo. In one aspect, the dsRNA molecules can be used to generate a loss-of-function mutation in a cell, an organ or an animal. Methods for making and using dsRNA molecules to selectively degrade RNA are well known in the art; see, for example, U.S. Pat. Nos. 6,489,127; 6,506,559; 6,511,824; and 6,515,109.

The present invention further relates to a mutant cell of a parent cell that comprises a disruption or deletion of a polynucleotide encoding the cytosine deaminase polypeptide or a control sequence thereof or a silenced gene encoding the polypeptide, which results in the mutant cell producing less of the cytosine deaminase or no cytosine deaminase compared to the parent cell.

The cytosine deaminase-deficient mutant cells are particularly useful as host cells for transformation with genes encoding native and heterologous proteins of interest. Therefore, the present invention further relates to methods of producing a native or heterologous polypeptide, comprising: (a) cultivating the mutant cell under conditions conducive for production of the polypeptide; and (b) recovering the polypeptide. The term “heterologous polypeptides” means polypeptides that are not native to the host cell, e.g., a variant of a native protein. The host cell may comprise more than one copy of a polynucleotide encoding the native or heterologous polypeptide.

The methods used for cultivation and purification of the product of interest may be performed by methods known in the art.

### EXAMPLES

Molecular cloning techniques are described in Sambrook, J., Fritsch, E. F., Maniatis, T. (1989) Molecular cloning: a laboratory manual (2nd edn.) Cold Spring Harbor Laboratory, Cold Spring Harbor, N.Y.

### Enzymes

Enzymes for DNA manipulations (e.g. restriction endonucleases, ligases etc.) are obtainable from New England Biolabs, Inc. and were used according to the manufacturer's instructions.

### Media and Reagents

Chemicals used for buffers and substrates were commercial products of analytical grade.

Cove: 342.3 g/L Sucrose, 20 ml/L COVE salt solution, 10 mM Acetamide, 30 g/L noble agar.

Cove top agar: 342.3 g/L Sucrose, 20 ml/L COVE salt solution, 10 mM Acetamide, 10 g/L low melt agarose

Cove-2: 30 g/L Sucrose, 20 ml/L COVE salt solution, 10 mM Acetamide, 30 g/L noble agar.

Cove-N(tf) plates are composed of 342.3 g sucrose, 20 ml Cove salt solution, 3 g NaNO3, and 30 g noble agar and water to 1 litre.

Cove-N plates are composed of 30 g sucrose, 20 ml Cove salt solution, 3 g NaNO3, and 30 g noble agar and water to 1 litre.

COVE salt solution is composed of 26 g KCl, 26 g MgSO4.7H2O, 76 g KH2PO4 and 50 ml Cove trace metals and water to 1 litre.

Trace metal solution for COVE is composed of 0.04 g NaB4O7.10H2O, 0.4 g CuSO4.5H2O, 1.2 g FeSO4.7H2O, 1.0 g MnSO4.H2O, 0.8 g Neutral amylase II MoO2.2H2O, and 10.0 g ZnSO4.7H2O and water to 1 litre.

Cove-N top agarose is composed of 342.3 g Sucrose, 20 ml COVE salt solution, 3 g NaNO3, and 10 g low melt agarose and water to 1 litre.

amyloglycosidase trace metal solution is composed of 6.8 g ZnCl2.7H2O, 2.5 g CuSO4.5H2O, 0.24 g NiCl2.6H2O, 13.9 g FeSO4.7H2O, 13.5 g MnSO4.H2O and 3 g citric acid, water to 1 litre.

YPG is composed of 4 g yeast extract, 1 g of KH2PO4, 0.5 g MgSO4.7H2O and 15 g Glucose (pH 6.0) and water to 1 litre.

STC buffer is composed of 0.8 M sorbitol, 25 mM Tris (pH 8), and 25 mM CaCl2 and water to 1 litre.

STPC buffer is composed of 40% PEG4000 in STC buffer.

MLC is composed of 40 g Glucose, 50 g Soybean powder, 4 g/Citric acid (pH 5.0) and water to 1 litre.

MSS is composed of 70 g Sucrose, 100 g Soybean powder (pH 6.0), and water to 1 litre.

MU-1 is composed 260 g Maltodextrin, 3 g MgSO4.7H2O, 5 g KH2PO4, 6 g of K2SO4, amyloglycosidase trace metal solution 0.5 ml and urea 2 g (pH 4.5) and water to 1 litre.

KCl plates are composed of 0.6M KCl, 20 ml of Cove salt solution, 3 g of NaNO3, and 30 g of noble agar and water to 1 litre.

5-fluorocytosine stock solution: 1000 mg 5-fluorocytosine dissolved in 1 ml 0.91 NaCl solution.

### Purchased Material (E. Coli, Plasmid and Kits)

E. coli DH5-alpha (Toyobo) is used for plasmid construction and amplification. The commercial plasmids/vectors TOPO cloning kit (Invitrogen) and pBluescript II SK- (Stratagene #212206) are used for cloning of PCR fragments. Amplified plasmids are recovered with Qiagen® Plasmid Kit (Qiagen). Ligation is done with DNA ligation kit (Takara) or T4 DNA ligase (Boehringer Mannheim). Polymerase Chain Reaction (PCR) is carried out with Expand TM PCR system (Boehringer Mannheim). QIAquick™ Gel Extraction Kit (Qiagen) is used for the purification of PCR fragments and extraction of DNA fragment from agarose gel.

### Strains

Aspergillus oryzae BECh-2 is described in Danish patent application PA 1999 01726. Aspergillus nidulans strain NRRL 1092 was used as a donor strain.

The expression host strain Aspergillus niger NN059095 was isolated by Novozymes and is a derivative of Aspergillus niger NN049184 which was isolated from soil. NN059095 was genetically modified to disrupt expression of amyloglycosidase activities.

Aspergillus oryzae ToC1512 is described in WO2005/070962, example 11.

### Plasmids

The expression plasmid pHUda440 and the nucleotide sequences of amyloglucosidase from Trametes cingulata are described in patent application WO2006/069289.

Plasmid pJaL574 and the nucleotide sequences of herpes simplex virus (HSV) thymidine kinase gene (TK), A. nidulans glyceraldehyde-3-phosphate dehydrogenase promoter (Pgpd) and A. nidulans tryptophane synthase terminator (TtrpC) are described in example 9 in WO07045248.

The expression cassette plasmid pJaL790 and the nucleotide sequences of neutral amylase II promoter (Pna2) is described in patent publication WO2005070962.

The JA126 amylase expression vector is described in patent application 10729.000-US.

Plasmid pDV8 is described in patent WO 2001/068864, example 8.

Plasmid pJaL504 is described in example 10.

Plasmid pJaL504-delta-BgIII is described in example 10.

Plasmid pJaL554 is described in patent WO2000/050567A1, example 1.

Plasmid pJaL574 is described in example 10.

Plasmid pJaL835 is described in example 10.

Plasmid pJaL955 is described in example 10.

Plasmid pJaL1022 is described in example 10.

Plasmid pJaL1025 is described in example 10.

Plasmid pJaL1027 is described in example 10.

Plasmid pJaL1029 is described in example 10.

Plasmid pJaL1120 is described in example 10.

Plasmid pJaL1123 is described in example 10.

Plasmid pJaL1183 is described in example 10.

Plasmid pJaL1194 is described in example 10.

Plasmid pJaL1202 is described in example 10.

Plasmid pToC65 is described in patent WO 91/17243

Plasmid pUC19: The construction is described in Vieira et al, 1982, Gene 19:259-268.

Plasmid pCR®4Blunt TOPO® from Invitrogen

### Transformation of Aspergillus

Transformation of Aspergillus species can be achieved using the general methods for yeast transformation. The preferred procedure for the invention is described below.

Aspergillus niger host strain was inoculated into 100 ml YPG medium supplemented with 10 mM uridine and incubated for 16 hrs at 32° C. at 80 rpm. Pellets were collected and washed with 0.6 M KCl, and resuspended in 20 ml 0.6 M KCl containing a commercial 6-glucanase product (GLUCANEX™, Novozymes A/S, Bagsværd, Denmark) at a final concentration of 20 mg per ml. The suspension was incubated at 32° C. with shaking (80 rpm) until protoplasts were formed, and then washed twice with STC buffer. The protoplasts were counted with a hematometer and resuspended and adjusted in an 8:2:0.1 solution of STC:STPC:DMSO to a final concentration of 2.5×107 protoplasts/ml. Approximately 4 μg of plasmid DNA was added to 100 μl of the protoplast suspension, mixed gently, and incubated on ice for 30 minutes. One ml of SPTC was added and the protoplast suspension was incubated for 20 minutes at 37° C. After the addition of 10 ml of 50° C. Cove or Cove-N top agarose, the reaction was poured onto Cove or Cove-N (tf) agar plates and the plates were incubated at 32° C. for 5 days.

### PCR Amplification

| | |
|---|---|
| 5×PCR buffer (incl. MgCl2) | 20 μl |
| 2.5 mM dNTP mix | 10 μl |
| Forward primer (100 μM) | 1 μl |
| Reverse primer (100 μM) | 1 μl |
| Expand High Fidelity polymerase (Roche) | 1 μl |
| Template DNA (50-100 ng/μl) | 1 μl |
| Distilled water to | 100 μl |

### PCR Conditions

| | | | |
|---|---|---|---|
| 94 C. | 2 min | | 1 cycle |
| 92 C. | 1 min | | |
| 55 C. | 1 min | {close oversize brace} | 30 cycles |
| 72 C. | 1-2 min | | |
| 72 C. | 7 min | | 1 cycle |

### SF Cultivation for Glucoamylase Production

Spores of the selected transformants were inoculated in 100 ml MLC media and cultivated at 30° C. for 2 days. 10 ml of MLC was inoculated to 100 ml of MU-1 medium and cultivated at 30° C. for 7 days. The supernatant was obtained by centrifugation.

### Southern Hybridization

Mycelia of the selected transformants were harvested from overnight culture in 100 ml YPG medium, rinsed with distilled water, dried and frozen at −80° C. Ground mycelia were incubated with Proteinase K and RNaseA at 65° C. for 1 hrs. Genome DNA was recovered by phenol/CHCl3 extraction twice followed by EtOH precipitation and resuspended in distilled water.

Non-radioactive probes were synthesized using a PCR DIG probe synthesis kit (Roche Applied Science, Indianapolis Ind.) followed by manufacture's instruction. DIG labeled probes were gel purified using a QIAquick™ Gel Extraction Kit (QIAGEN Inc., Valencia, Calif.) according to the manufacturer's instructions.

Five micrograms of genome DNA was digested with appropriate restriction enzymes completely for 16 hours (40 μl total volumes, 4 U enzyme/μl DNA) and run on a 0.8% agarose gel. The DNA was fragmented in the gel by treating with 0.2 M HCl, denatured (0.5 M NaOH, 1.5 M NaCl) and neutralized (1 M Tris, pH7.5; 1.5 M NaCl) for subsequent transfer in 20×SSC to Hybond N+ membrane (Amersham). The DNA was UV cross-linked to the membrane and prehybridized for 1 hour at 42oC in 20 ml DIG Easy Hyb (Roche Diagnostics Corporation, Mannheim, Germany). The denatured probe was added directly to the DIG Easy Hyb buffer and an overnight hybridization at 42oC was done. Following the post hybridization washes (twice in 2×SSC, room temperature, 5 min and twice in 0.1×SSC, 68o C, 15 min. each), chemiluminescent detection using the DIG detection system and CPD-Star (Roche) was done followed by manufacture's protocol. The DIG-labeled DNA Molecular Weight Marker II (Roche) was used for the standard marker.

### Glucoamylase Activity

Glucoamylase activity is measured in AmyloGlucosidase Units (AGU). The AGU is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes. An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

### Amyloglycosidase Incubation:

Substrate: maltose 23.2 mM

Buffer: acetate 0.1 M

pH: 4.30±0.05

Incubation temperature: 37° C.±1

Reaction time: 5 minutes

Enzyme working range: 0.5-4.0 AGU/mL

### Color Reaction:

GlucDH: 430 U/L

Mutarotase: 9 U/L

NAD: 0.21 mM

Buffer: phosphate 0.12 M; 0.15 M NaCl

pH: 7.60±0.05

Incubation temperature: 37° C.±1

Reaction time: 5 minutes

Wavelength: 340 nm

### Determination of Acid Alpha-Amylase Activity

When used according to the present invention the activity of any acid alpha-amylase may be measured in AFAU (Acid Fungal Alpha-amylase Units), which are determined relative to an enzyme standard. 1 FAU is defined as the amount of enzyme which degrades 5.260 mg starch dry matter per hour under the below mentioned standard conditions.

Acid alpha-amylase, i.e., acid stable alpha-amylase, an endo-alpha-amylase (1,4-alpha-D-glucan-glucano-hydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

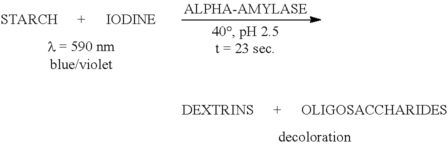

### Standard Conditions/Reaction Conditions:

Substrate: Soluble starch, approx. 0.17 g/L

Buffer: Citrate, approx. 0.03 M

Iodine (I2): 0.03 g/L

CaCl2: 1.85 mM

pH: 2.50±0.05

Incubation temperature: 40° C.

Reaction time: 23 seconds

Wavelength: 590 nm

Enzyme concentration: 0.025 AFAU/mL

Enzyme working range: 0.01-0.04 AFAU/mL

### Example 1

### Introduction of FRT Sites at the Neutral Amylase I (NAI) Locus in Aspergillus Niger NN059095

Construction of Hygromycin B Resistance Gene Expression Plasmid pHUda966

The following primers Tef-F and Tef-R which introduce EcoRI/SpeI and a BamHI site, respectively, were designed to isolate a promoter region of A. oryzae tef1 (translation elongation factor 1/Ptef1) based on the nucleotide sequences information in GENBANK (ID#AB007770):

Tef-F:
(SEQ ID NO: 1)
gaattcactagtggggttcaaatgcaaacaa
Tef-R:
(SEQ ID NO: 2)
ggatcctggtgcgaactttgtagtt

A PCR reaction with the genome DNA of the Aspergillus oryzae strain BECh2 as template was performed using a primer pair of Tef-F and Tef-R. The reaction products were isolated on a 1.0% agarose gel and 0.7 kb product band was excised from the gel. The 0.7 kb amplified DNA fragment was digested with BamHI and EcoRI, and ligated into the Aspergillus expression cassette pHUda440 digested with BamH I and EcoRI to create pHUda440-Ptef.

The following primers nia-F and nia-R which introduce an XhoI and an XbaI site, respectively, were designed to isolate a terminator region of A. oryzae nitrate reductase (niaD) (Tniad) based on the nucleotide sequences information in EMBL:D49701:

nia-F:
(SEQ ID NO: 3)
ctcgagattatccaagggaatgac
nia-R:
(SEQ ID NO: 4)
tctagaaagtattttcggtacgatt

A PCR reaction with the genome DNA of the Aspergillus oryzae strain BECh2 as template was performed using a primer pair of nia-F and nia-R. The reaction products were isolated on a 1.0% agarose gel and 0.5 kb product band was excised from the gel. The 0.5 kb amplified DNA fragment was digested with XhoI and XbaI, and ligated into the Aspergillus expression cassette pHUda440-Ptef digested with XhoI and XbaI to create pHUda440-Ptef-Tnia.

The following primers hph-F and hph-R which introduce a BamH and an XhoI site, respectively, were designed to isolate a coding region of hygromycin B resistance gene based on the nucleotide sequences information in EMBL:AR109978:

hph-F:
(SEQ ID NO: 5)
ggatcctacacctcagcaatgtcgcctgaa
hph-R:
(SEQ ID NO: 6)
ctcgagctattcctttgccctcggacgagtgct

A PCR reaction with pJaL154 harboring the hygromycin B resistance gene (hph) as template was performed using a primer pair of hph-F and hph-R. The reaction products were isolated on a 1.0% agarose gel and 1.0 kb product band was excised from the gel. The 1.0 kb amplified DNA fragment was digested with BamHI and XhoI, and ligated into the Aspergillus expression cassette pHUda440-Ptef-Tnia digested with BamHI and XhoI to create pHUda966. The nucleotide sequences of hygromycin B resistance gene (hph) expression parts in pHUda966 are shown in SEQ ID NO:7, with indications of the features positions of the primers used for the construction, the encoded hygromycin B resistance factor is shown in SEQ ID NO:8.

Construction of pHUda981 for Introduction of FRT Sites at the NA1 Loci

The 2.5 kb DNA fragment containing herpes simplex virus (HSV) thymidine kinase gene (TK) was recovered from pJaL574 by XhoI and EcoRI digestion. The recovered 2.5 kb fragment was ligated to XhoI and EcoRI digested pBluescript II SK-. The ligation mixture was transformed into E. coli DH5α to create the expression plasmid pTK.

The nucleotide sequences of the FRT-F and FRT-F3 sites are:

FRT-F:
(SEQ ID NO: 9)
ttgaagttcctattccgagttcctattctctagaaagtataggaacttc
FRT-F3:
(SEQ ID NO: 10)
ttgaagttcctattccgagttcctattcttcaaatagtataggaacttca

The following primers 3NA1-F and 3NA1-R which introduce an EcoRI and a SpeI site, respectively, were designed to isolate 3′ flanking region of Aspergillus niger neutral amylase I (NAI) fused with FRT-F3 recognition site based on the nucleotide sequences information in EMBL:AM270106 and EMBL: DJ052242, respectively:

3NA1-F:
(SEQ ID NO: 11)
actagtttgaagttcctattccgagttcctattcttcaaatagtataggaacttcaactag
agtatatgatggtact
3NA1-R:
(SEQ ID NO: 12)
gaattcgcattctcctagttactgatgacttt

A PCR reaction with the genome DNA of Aspergillus niger NN059095 as template was performed using a primer pair of 3NA1-F and 3NA1-R. The reaction products were isolated on a 1.0% agarose gel and 1.0 kb product band was excised from the gel. The 1.5 kb amplified DNA fragment was digested with SpeI and EcoRI, and ligated into the Aspergillus expression cassette pTK digested with EcoRI and SpeI to create pHUdaTK-3NA1.

The following primers 5NA1-F and 5NA1-R which introduce a NotI and a SpeI site, respectively, were designed to isolate 5′ flanking region of Aspergillus niger neutral amylase I (NAI) fused with FRT-F recognition site based on the nucleotide sequences information in EMBL:AM270106 and EMBL: DJ052242, respectively:

5NA1-F:
(SEQ ID NO: 13)
gcggccgcgtttaaacctatctgttccc
5NA1-R:
(SEQ ID NO: 14)
actagtgctagcgaagttcctatactttctagagaataggaactcggaataggaacttcaag
atgaattcgcggcctacatg

A PCR reaction with the genome DNA of Aspergillus niger NN059095 as template was performed using a primer pair of 5NA1-F and 5NA1-R. The reaction products were isolated on a 1.0% agarose gel and 1.8 kb product band was excised from the gel. The 1.8 kb amplified DNA fragment was digested with NotI and SpeI, and ligated into the Aspergillus expression cassette pTK-3NA1 digested with NotI and SpeI to create pHUdaTK-3NA1-5NA1.

The 2.2 kb DNA fragment containing hybromycin B resistance gene driven by Aspergillus oryzae tef1 promoter (Ptef) and niaD terminator (Tniad) was recovered from pHUda966 by XbaI and NheI digestion. The recovered 2.2 kb fragment was ligated to SpeI digested pHUdaTK-3NA1-5NA1. The ligation mixture was transformed into E. coli DH5α to create the expression plasmid pHUda981.

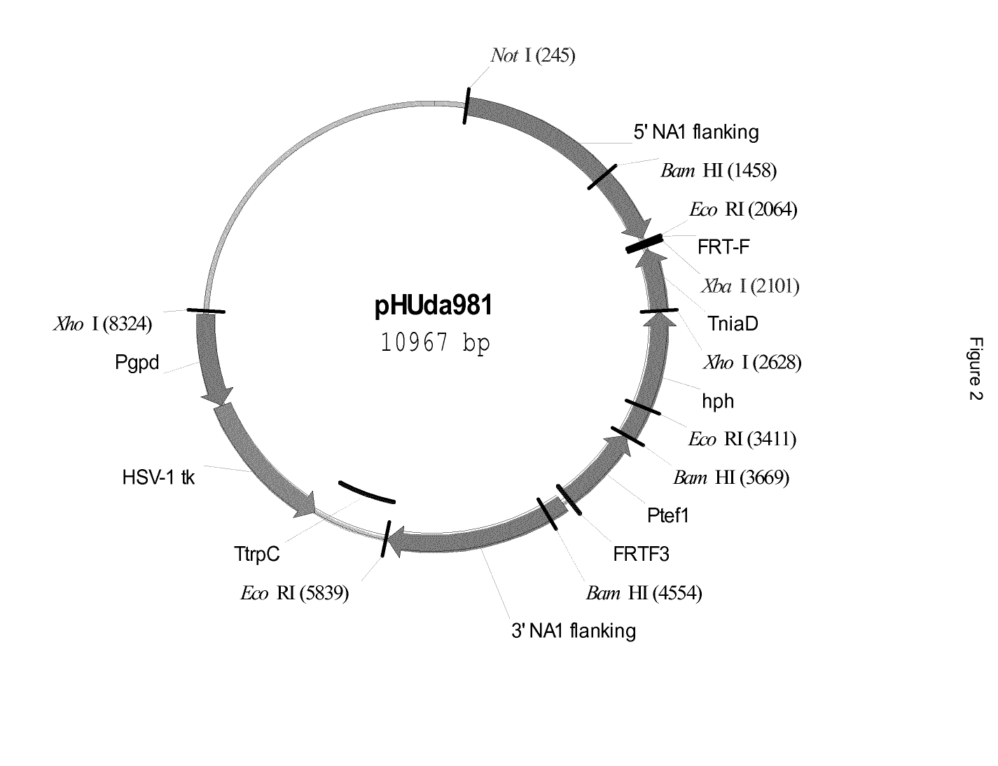

The nucleotide sequence of the NA1-encoding part and flanking regions of pHUda981 is shown in SEQ ID NO:15, the NA1 is shown in SEQ ID NO: 16 and a plasmid map is shown in FIG. 2.

### Introduction of FRT Sites at the NA1 Locus in A. Niger NN059095

The pHUda981 was introduced into Aspergillus niger strain NN059095. Transformants were selected from the Cove-N (tf) supplemented with 10 mM uridine and 1 mM hygromycin B. Randomly selected transformants were inoculated onto Cove-N plates with 10 mM uridine, 1 mM hygromycin B and 2.5 μM 5-Fluoro-2-deoxyuridine (FdU), an agent which kills cells expressing the herpes simplex virus (HSV) thymidine kinase gene (TK) harbouring in pHUda981. Strains which grew well on Cove-N plates supplemented with 2.5 μM FdU were purified and subjected to Southern blotting analysis to confirm whether the FRT sites in pHUda981 was introduced correctly or not.

The following set of primers to make a non-radioactive probe was used to analyze the selected transformants. For the 5′ NA1 flanking region:

Forward primer:
(SEQ ID NO: 17)
aatccggatcctttcctata
Reverse primer:
(SEQ ID NO: 18)
gatggagcgcgcctagaagc

Genomic DNA extracted from the selected transformants was digested by NcoI and Southern blotting analysis was preformed using the above probe. Strains of interest were identified by the disappearance of a 2.8 kb NcoI band and the appearance of a 3.1 kb NcoI band. Among the strains given the right integration events, a strain denoted NN059180 was selected.

### Example 2

### Introduction of FRT Sites at the Acid Stable Amylase Locus in A. Niger NN059095

Construction of A. Nidulans Acetoamidase Gene (amdS) Expression Plasmid pHUda976.

The following primers amdS-F and amdS-R which introduce a BamHI and an XhoI site, respectively, were designed to isolate a coding region of amdS gene based on the nucleotide sequences information in EMBL:AF348620:

amdS-F:
(SEQ ID NO: 19)
ggatccaccatgcctcaatcctgg
amdS-R:
(SEQ ID NO: 20)
ctcgagctatggagtcaccacatttcccag

A PCR reaction with genome DNA of Aspergillus nidulans strain NRRL 1092 as template was performed using a primer pair of amdS-F and amdS-R. The reaction products were isolated on a 1.0% agarose gel and 1.0 kb product band was excised from the gel. The 1.9 kb amplified DNA fragment was digested with BamHI and XhoI, and ligated into the Aspergillus expression cassette pHUda440-Ptef-Tnia digested with BamHI and XhoI to create pHUda976.

The nucleotide sequence of the Aspergillus nidulans acetoamidase gene (amdS) expression parts in pHUda976 is shown in SEQ ID NO:21 with gene features positions of the primers used, the encoded acetoamidase amino acid sequence is shown in SEQ ID NO:22.

Construction of pHUda1019 for Introduction of FRT Sites at the Acid Stable Amylase Locus

The following primers 3SP-F and 3SP-R which introduce an EcoRI and a SpeI site, respectively, were designed to isolate 3′ flanking region of Aspergillus niger acid stable amylase fused with FRT-F3 recognition site based on the nucleotide sequences information in EMBL:AM270232 and EMBL: DJ052242, respectively:

3SP-F:
(SEQ ID NO: 23)
actagtttgaagttcctattccgagttcctattcttcaaatagtataggaacttcaactagagaa
tgcaatcataacagaaagta
3SP-R:
(SEQ ID NO: 24)
gaattcttaattaaatcacggcaagggtttac

A PCR reaction with the genome DNA of Aspergillus niger NN059095 as template was performed using a primer pair of 3SP-F and 3SP-R. The reaction products were isolated on a 1.0% agarose gel and 1.8 kb product band was excised from the gel. The 1.8 kb amplified DNA fragment was digested with SpeI and EcoRI, and ligated into the Aspergillus expression cassette pTK digested with EcoRI and SpeI to create pHUdaTK-3SP.

The following primers 5SP-F and 5SP-R which introduce a SacII and a SpeI site, respectively, were designed to isolate 5′ flanking region of Aspergillus niger acid stable amylase fused with FRT-F recognition site based on the nucleotide sequences information in EMBL:AM270232 and EMBL: DJ052242, respectively:

5SP-F:
(SEQ ID NO: 25)
ccgcggcaacaggcagaatatcttcc
5SP-R:
(SEQ ID NO: 26)
actagtgaagttcctatactttctagagaataggaactcggaataggaacttcaaacggg
atcttggacgcattcca

A PCR reaction with the genome DNA of Aspergillus niger NN059095 as template was performed using a primer pair of 5SP-F and 5SP-R. The reaction products were isolated on a 1.0% agarose gel and 2.0 kb product band was excised from the gel. The 2.0 kb amplified DNA fragment was digested with SacII and SpeI, and ligated into the Aspergillus expression cassette pTK-3SP digested with SacII and SpeI to create pHUdaTK-3SP-5SP.

The 3.1 kb DNA fragment containing the amdS gene driven by Aspergillus oryzae tef1 promoter and niaD terminator was recovered from pHUda976 by XbaI and NheI digestion. The recovered 3.1 kb fragment was ligated to SpeI digested pHUdaTK-3SP-5SP. The ligation mixture was transformed into E. coli DH5α to create the expression plasmid pHUda1019.

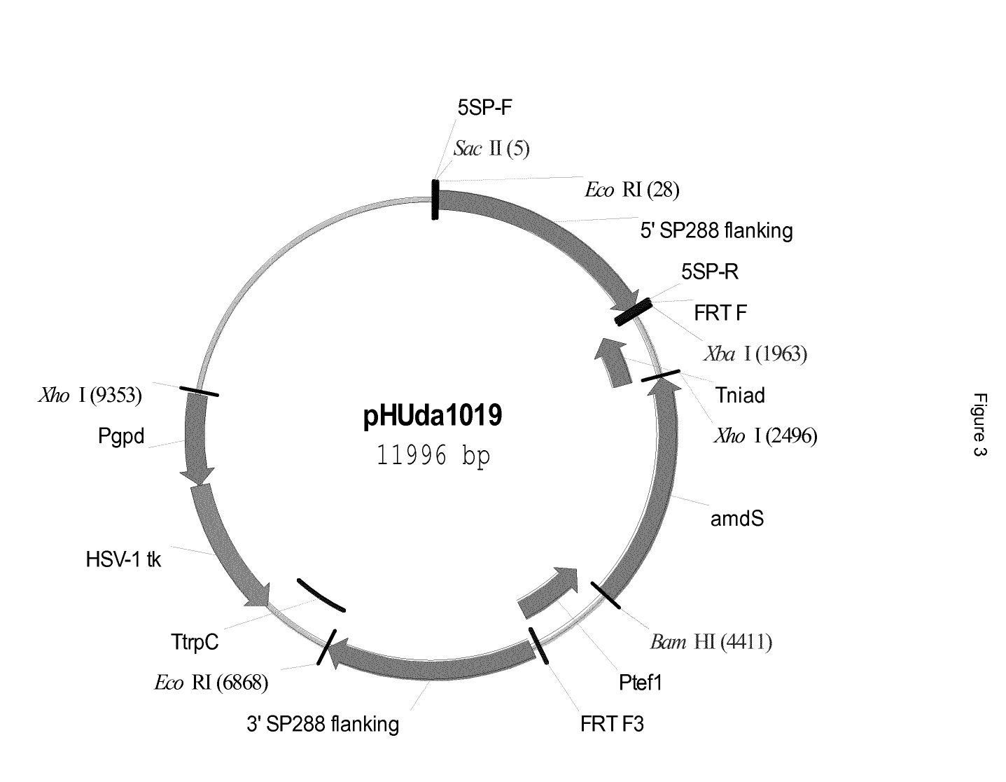

The nucleotide sequence of the A. niger acid stable amylase gene with the flanking sequences of pHUda1019 are shown in SEQ ID NO:27 and the encoded amylase amino acid sequence is shown in SEQ ID NO:28; a plasmid map is shown in FIG. 3.

### Introduction of FRT Sites at the Locus in A. Niger NN059180

The pHUda1019 was introduced into Aspergillus niger strain NN059180. Transformants were selected from the Cove (tf) supplemented with 10 mM uridine. Randomly selected transformants were inoculated onto Cove-2 plates with 10 mM uridine and 2.5 μM 5-Fluoro-2-deoxyuridine (FdU), an agent which kills cells expressing the herpes simplex virus (HSV) thymidine kinase gene (TK) harbouring in pHUda1019. Strains which grew well on Cove-2 plates with 2.5 μM FdU were purified and subjected to Southern blotting analysis to confirm whether the FRT sites in pHUda1019 was introduced correctly or not.

The following set of primers to make non-radioactive probe was used to analyze the selected transformants. For 5′ acid stable amylase flanking region:

Forward primer:
(SEQ ID NO: 29)
cgtacaccttgggattatgcgctg
Reverse primer:
(SEQ ID NO: 30)
cacaaaggcgcaaagcataccatc

Genomic DNA extracted from the selected transformants was digested by XhoI. The right integration event were identified by the disappearance of a 6.2 kb XhoI band and the appearance of a 4.1 XhoI band band. Among the strains given the right integration events, a strain denoted NN059183 was selected.

### Example 3

### Simultaneous Site Specific-Integration by FLP in the Two Loci

Construction of A. Nidulans pyrG Gene Expression Plasmid pHUda794

The following primers pyr-F introducing a PacI site and pyr-R were designed to isolate a promoter and coding region of A. nidulans pyrG gene based on the nucleotide sequences information in EMBL:m19132:

pyr-F:
(SEQ ID NO: 31)
ttaattaaactaaatgacgtttgtgaaca
pyr-R:
(SEQ ID NO: 32)
ctaccgccaggtgtcagtcaccctcaaagtccaactcttttc

The following primers Tamg-F and Tamg-R introducing a SphI site were designed to isolate a terminator region of A. niger amyloglucosidase (Tamg) gene fused with FRT-F3 recognition site based on the nucleotide sequences information in EMBL:am270061 and DJ052242:

Tamg-F:
(SEQ ID NO: 33)
agagttggactttgagggtgactgacacctggcggtag
Tamg-R:
(SEQ ID NO: 34)
gcatgcactagctagttgaagttcctatactatttgaagaatagg
aactcggaataggaacttcaacctagaggagagagttg

A PCR reaction with genome DNA of Aspergillus nidulans strain NRRL 1092 as template was performed using a primer pair of pyr-F and pyr-R. The reaction products were isolated on a 1.0% agarose gel and 1.4 kb product band was excised from the gel.

A PCR reaction with the genome DNA of Aspergillus niger NN059095 as template was performed using a primer pair of Tamg-F and Tamg-R. The reaction products were isolated on a 1.0% agarose gel and 0.8 kb product band was excised from the gel.

A PCR reaction with the 1.4 kb and 0.8 kb amplified DNA fragment was performed using a primer pair of pyr-F and Tamg-R. The reaction products were isolated on a 1.0% agarose gel and 2.2 kb product band was excised from the gel.

The 2.2 kb amplified DNA fragment was packed into the TOPO cloning vector (pCR2.1 TOPO) provided by Invitrogen followed by the protocol with the kit to create pHUda794.

The nucleotide sequence of the A. nidulans pyrG gene with flanking sequences in pHUda794 is shown in SEQ ID NO:35 along with features and positions of primers used; the amino acid sequence of the encoded PyrG is shown in SEQ ID NO:36.

Construction of Synthetic Version of FLP Gene Expression Plasmid pHUda996

The following primers xIn-F and xIn-R introducing a SphI site and a BamHI, respectively, were designed to isolate a promoter region of A. nidulans xInA gene (PxInA) based on the nucleotide sequences information in EMBL:z49892:

xln-F:
(SEQ ID NO: 37)
gcatgcttaattaatggaagtgcgttgatcatt
xln-R:
(SEQ ID NO: 38)
ggatcccctgtcagttggg

A PCR reaction with genome DNA of Aspergillus nidulans strain NRRL 1092 as template was performed using a primer pair of xIn-F and xIn-R. The reaction products were isolated on a 1.0% agarose gel and 0.7 kb product band was excised from the gel. The 0.7 kb amplified DNA fragment was digested with BamHI and SphI, and ligated into the Aspergillus expression cassette pHUda966 digested with BamHI and SphI to create pHUda966-PxInA.

The 1.3 kb DNA fragment containing synthetic version of FLP gene (sFLP) was recovered from pJaL1008 by BamHI and XhoI digestion. The recovered 1.3 kb fragment was ligated to BamHI and XhoI digested pHUda966-PxInA. The ligation mixture was transformed into E. coli DH5α to create the expression plasmid pHUda996.

The nucleotide sequences of the synthetic version of FLP expression parts in pHUda996 is shown in SEQ ID NO:39 together with features and positions of the primers used; the amino acid sequence of the encoded sFLP is shown in SEQ ID NO:40.

Construction of pHUda1000 for Simultaneous Site Specific-Integration at the Neutral Amylase 1 (NA1) and the Acid Stable Amylase Loci in NN059183

The following primers Pna-F and Pna-R introducing an EcoRI site and a BamHI site, respectively, were designed to isolate a promoter region of A. niger neutral amylase II (NA2) gene (Pna2) put triple in tandem fused with FRT-F recognition site based on the nucleotide sequences information in pJaL790 and EMBL:DJ052242:

Pna-F:
(SEQ ID NO: 41)
gaattcatcttgaagttcctattccgagttcctattctctagaaagtataggaacttcgcta
gccgagagcagcttgaaga
Pna-R:
(SEQ ID NO: 42)
ggatcccccagttgtgtatatagaggatt

A PCR reaction with pJaL790 as template was performed using a primer pair of Pna-F and Pna-R. The reaction products were isolated on a 1.0% agarose gel and 1.7 kb product band was excised from the gel. The 1.7 kb amplified DNA fragment was digested with EcoRI and BamHI, and ligated into the Aspergillus expression cassette pHUda440 harboring amyloglucosidase gene from Trametes cingulata (T.c. GA) digested with EcoRI and BamHI to create pHUda440-FRT.

The 2.2 kb DNA fragment containing A. nidulans pyrG gene was recovered from pHUda794 by PacI and SphI digestion. The recovered 2.2 kb fragment was ligated to PacI and SphI digested pHUda440-FRT. The ligation mixture was transformed into E. coli DH5α to create the expression plasmid pHUda440-FRT-pyrG.

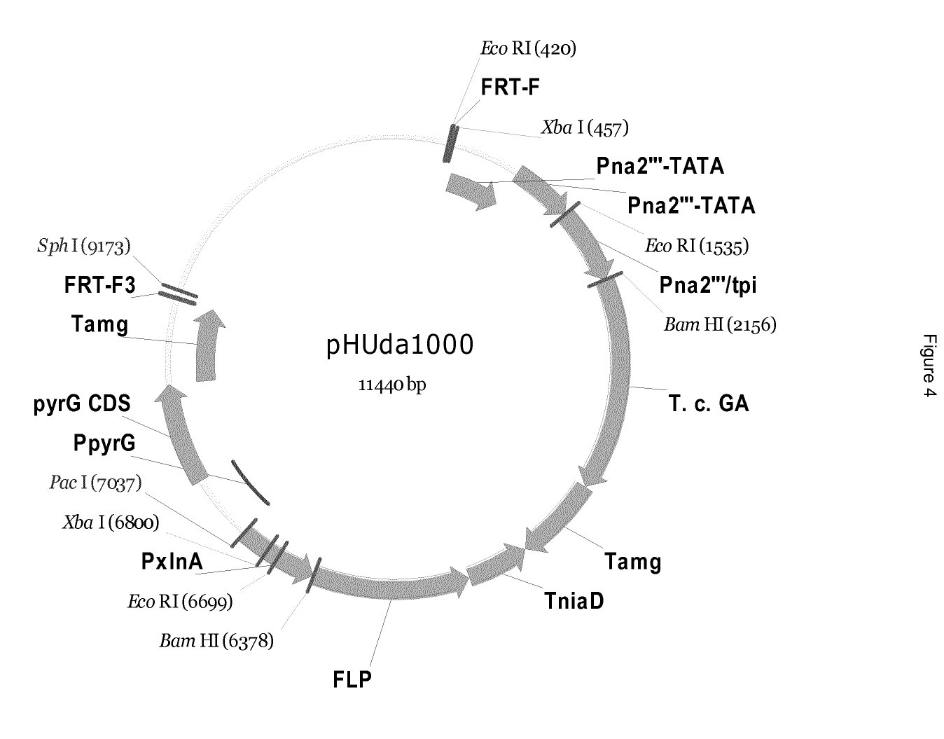

The 2.4 kb DNA fragment containing FLP gene driven by xInA promoter and niaD terminator was recovered from pHUda996 by PacI and XbaI digestion. The recovered 2.4 kb fragment was ligated to PacI and XbaI digested pHUda440-FRT-pyrG. The ligation mixture was transformed into E. coli DH5α to create the expression plasmid pHUda1000. A plasmid map is shown in FIG. 4.

### Simultaneous Site Specific-Integration by FLP

The pHUda1000 was introduced into Aspergillus niger strain NN059183. Transformants were selected from the Cove-N (tf) supplemented with 1% D-xylose. Randomly selected transformants were inoculated onto Cove-N plates. Strains which grew well on Cove-N plates were purified and subjected to Southern blotting analysis to confirm whether the expression part in pHUda1000 was introduced correctly or not.

The following set of primers to make a non-radioactive probe was used to analyze the selected transformants. For T.c.GA coding region:

Forward primer:
(SEQ ID NO: 43)
tcgagtgcggccgacgcgtacgtc
Reverse primer:
(SEQ ID NO: 44)
cagagagtgttggtcacgta

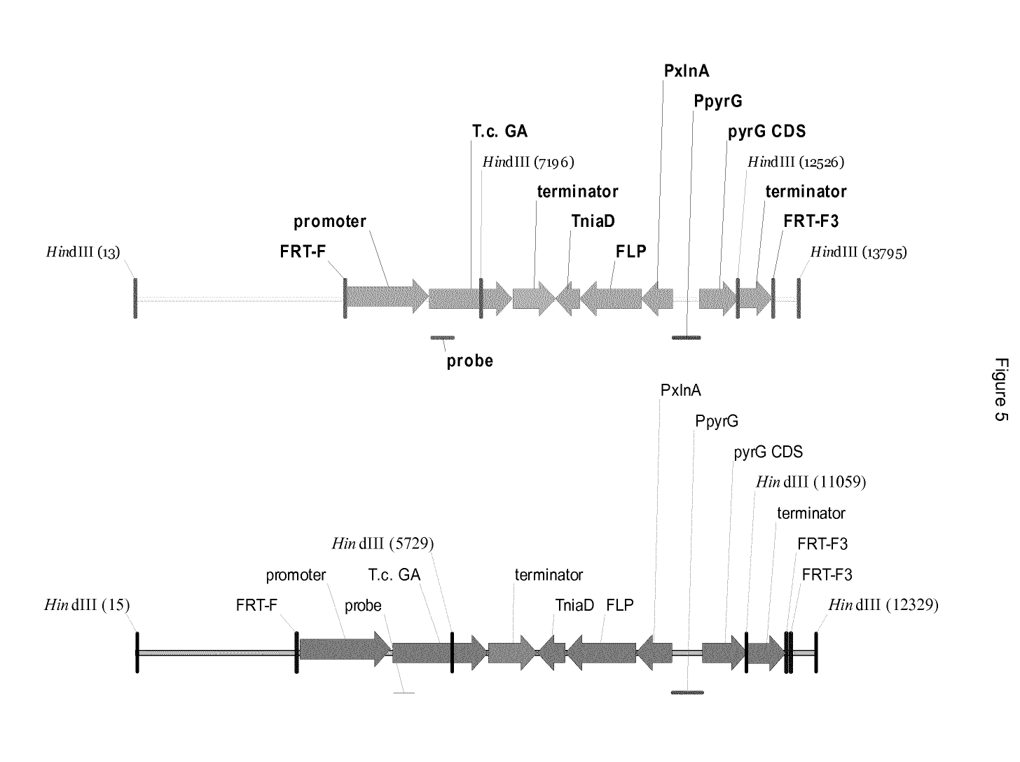

Genomic DNA extracted from the selected transformants was digested by HindIII and Southern blotting analysis was preformed using the above probe. Strains of interest were identified by the disappearance of a 2.8 kb NcoI band and the appearance of a 3.1 kb NcoI band. By the right integration event, two hybridized signals of the size 7.2 kb and 5.7 kb introduced at NA1 and acid stable amylase loci, respectively, were seen. FIG. 5 shows the schematic NA1 (upper panel) and acid stable amylase loci (lower panel) when the pHUda1000 was introduced correctly in NN059183.

### Example 4

### A. niger ku70 Gene Disruption in NN059183

Construction of the A. Niger ku70 Gene Disruption Vector pHUda801

The following primers 3ku-F and 3ku-R introducing an EcoRI site and a SpeI site, respectively, were designed to isolate a 3′ flanking region of A. niger ku70 gene based on the nucleotide sequences information in EMBL:am270339:

3ku-F:
(SEQ ID NO: 45)
actagttctagaagccgtgggtatttttatgaa
3ku-R:
(SEQ ID NO: 46)
gaattcgtttaaacttggcggctgccaagcttcc

A PCR reaction with genome DNA of Aspergillus niger strain NN059183 as template was performed using a primer pair of 3ku-F and 3ku-R. The reaction products were isolated on a 1.0% agarose gel and 2.0 kb product band was excised from the gel. The 2.0 kb amplified DNA fragment was digested with EcoRI and SpeI, and ligated into the pTK digested with EcoRI and SpeI to create pTK-3ku.

The following primers 5ku-F and 5ku-R introducing a NotI site and a SpeI site, respectively, were designed to isolate a 5′ flanking region of A. niger ku70 gene based on the nucleotide sequences information in EMBL:am270339:

5ku-F:
(SEQ ID NO: 47)
gcggccgctcattcagagagctacccgt
5ku-R:
(SEQ ID NO: 48)
actagttaattaagaggaccgcatctttga

A PCR reaction with genome DNA of Aspergillus niger strain NN059183 as template was performed using a primer pair of 5ku-F and 5ku-R. The reaction products were isolated on a 1.0% agarose gel and 1.3 kb product band was excised from the gel. The 1.3 kb amplified DNA fragment was digested with NotI and SpeI, and ligated into the pTK-3ku digested with NotI and SpeI to create pTK-3ku-5ku.

The 2.2 kb DNA fragment containing A. nidulans pyrG gene was recovered from pHUda794 by SpeI and XbaI digestion. The recovered 2.2 kb fragment was ligated to SpeI and XbaI digested pTK-3ku-5ku. The ligation mixture was transformed into E. coli DH5α to create the expression plasmid pHUda801.

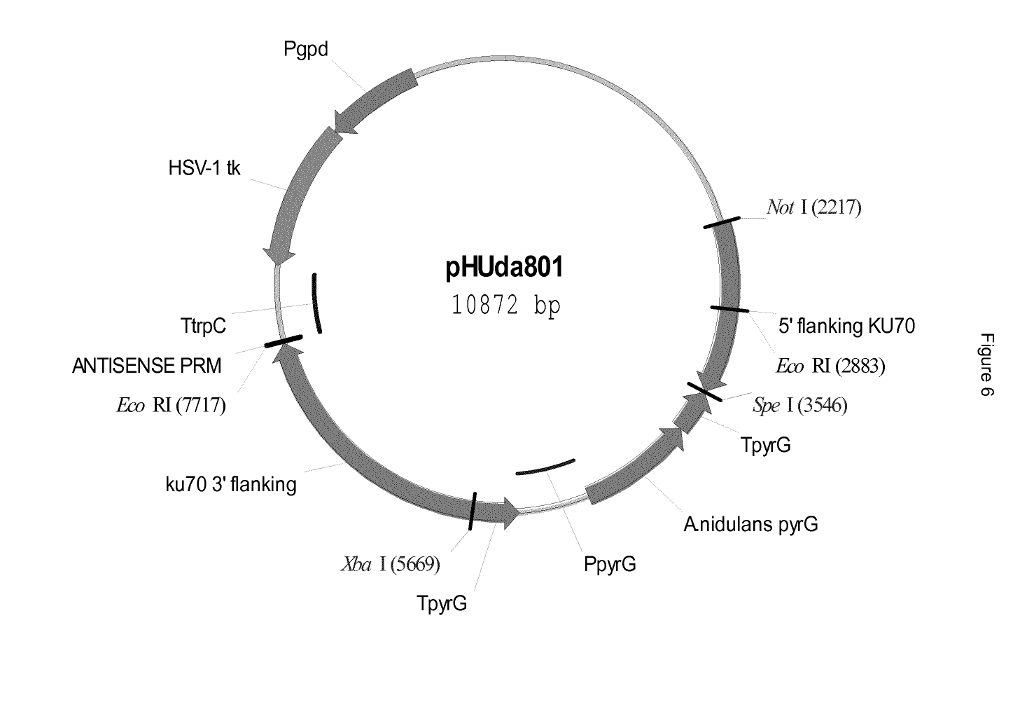

The nucleotide sequence of the A. niger ku70 gene and flanking sequences of pHUda801 are shown in SEQ ID NO:49; the amino acid sequence of the ku70-encoded polypeptide is shown in SEQ ID NO:50. A plasmid map is shown in FIG. 6.

The ku70 Gene Disruption in NN059183

The pHUda801 was introduced into Aspergillus niger strain NN059183. Transformants were selected from the Cove-N (tf). Randomly selected transformants were inoculated onto Cove-N plates with 2.5 μM 5-Fluoro-2-deoxyuridine (FdU), an agent which kills cells expressing the herpes simplex virus (HSV) thymidine kinase gene (TK) harboured in pHUda801. Strains which grew well on Cove-N plates with 2.5 μM FdU were purified and subjected to Southern blotting analysis to confirm whether the ku70 gene was disrupted correctly or not.

The following set of primers to make a non-radioactive probe was used to analyze the selected transformants. For the 3′ ku70 flanking region:

Forward primer:
(SEQ ID NO: 51)
acggtatgcgtacaatgatca
Reverse primer:
(SEQ ID NO: 52)
atttgagggcaccagcacccc

Genomic DNA extracted from the selected transformants was digested by SpeI. By the right gene disruption event, a hybridized signal of the size of 8.3 kb by SpeI digestion was shifted to 5.1 kb probed described above. Among the strains given the right integration events, a strain denoted C1997 was selected.

### Example 5

### Simultaneous Site Specific-Integration by FLP in the Two Loci in C1997

### PyrG Gene Rescue in C1997

At first, the introduced pyrG gene at the ku70 loci in C1997 was rescued as follows. The strain C1997 was inoculated once on Cove-N media containing 10 mM uridine and 1 g/L 5-fluoro-orotic acid (5-FOA). Strains in which the pyrG gene has been deleted will grow in the presence of 5-FOA; those that retain the gene will convert 5-FOA to 5-fluoro-UMP, a toxic intermediate. The colonies that grew more quickly were isolated. The isolated strain was named M1117.

### Simultaneous Site Specific-Integration by FLP in M1117

The pHUda1000 was introduced into Aspergillus niger strain M1117. Transformants were selected from the Cove-N (tf) supplemented with 1 g/L D-xylose. Randomly selected transformants were inoculated onto Cove-N plates. Strains which grew well on Cove-N plates were purified and subjected to Southern blotting analysis to confirm whether the expression part in pHUda1000 was introduced correctly or not.

The following set of primers to make a non-radioactive probe was used to analyze the selected transformants. For the T.c.GA coding region:

Forward primer:
(SEQ ID NO: 53)
tcgagtgcggccgacgcgtacgtc
Reverse primer:
(SEQ ID NO: 54)
cagagagtgttggtcacgta

Genomic DNA extracted from the selected transformants was digested by HindIII. By the right integration event, two hybridized signals at the size of 7.2 kb and 5.7 kb introduced at NA1 and acid stable amylase loci, respectively, were seen.

The frequency of the simultaneous integration with the ku70 gene disruption (M1117) was approx. 20% whereas that without ku70 gene disruption (NN059183) was around 4-5%. It suggested that the ku70 gene disruption played a great role in improving the locus specific integration frequency by FLP.

### Example 6

### A. Niger fcy1 Gene Disruption in NN059183

Construction of the A. Niger (Cytosine Deaminase) fcy1 Gene Disruption Vector pHUda1043

The following primers 3fcy-F and 3fcy-R introducing a XbaI site and a PmeI site, respectively, were designed to isolate a 3′ flanking region of A. niger fcy1 gene based on the nucleotide sequences information in EMBL:am269962:

3fcy-F:
(SEQ ID NO: 55)
tctagaattgaaagctagttctggtcgcat
3fcy-R:
(SEQ ID NO: 56)
gtttaaactccttgcttcgcatacatgcccac

A PCR reaction with genome DNA of Aspergillus niger strain NN059183 as template was performed using a primer pair of 3fcy-F and 3fcy-R. The reaction products were isolated on a 1.0% agarose gel and 2.0 kb product band was excised from the gel. The 2.0 kb amplified DNA fragment was digested with XbaI and PmeI, and ligated into the pHUda801 digested with XbaI and PmeI to create pHUda801-3fcy.

The following primers 5fcy-F and 5fcy-R introducing a NotI site and a SpeI site, respectively, were designed to isolate a 5′ flanking region of A. niger fcy1 gene based on the nucleotide sequences information in EMBL:am269962:

5fcy-F:
(SEQ ID NO: 57)
gcggccgccgccgccgaagaactgagcaaa
5fcy-R:
(SEQ ID NO: 58)
actagtatatcttcttatcgcagagattg

A PCR reaction with genome DNA of Aspergillus niger strain NN059183 as template was performed using a primer pair of 5fcy-F and 5fcy-R. The reaction products were isolated on a 1.0% agarose gel and 2.1 kb product band was excised from the gel. The 2.1 kb amplified DNA fragment was digested with NotI and SpeI, and ligated into the pHUda801-3fcy digested with NotI and SpeI to create pHUda1043.

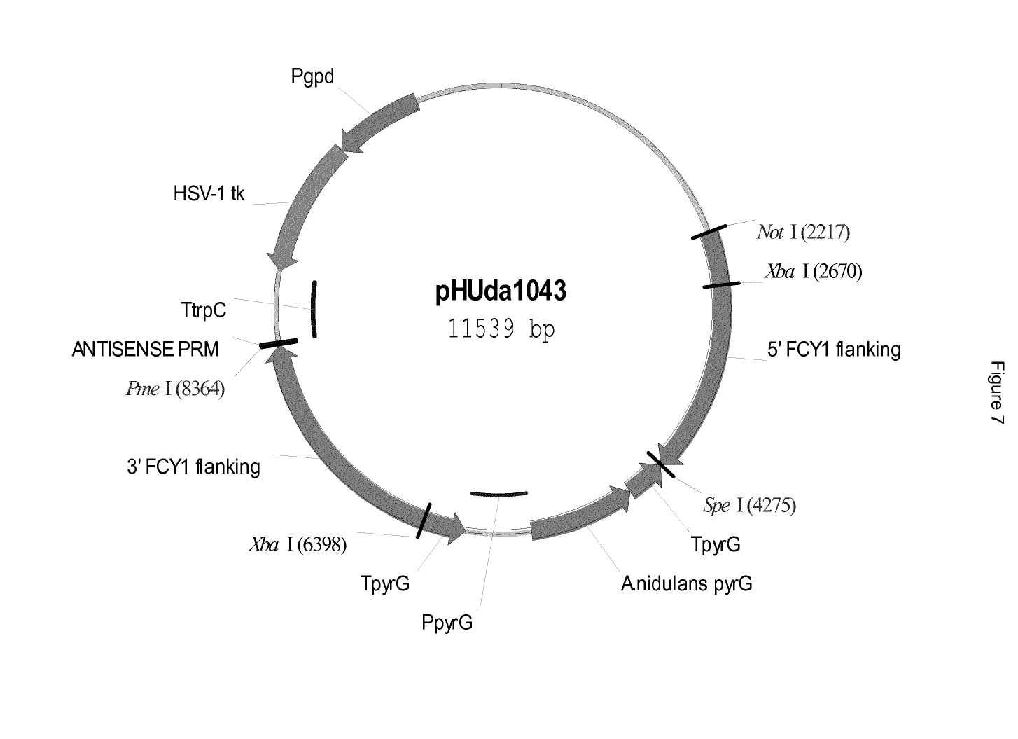

The nucleotide sequence of the A. niger fcy1 gene and flanking sequences in pHUda1043 is shown in SEQ ID NO:59; the amino acid sequence of the fcy1-encoded polypeptide is shown in SEQ ID NO:60. A plasmid map is shown in FIG. 7.

The fcy1 Gene Disruption in NN059183

The pHUda1043 was introduced into Aspergillus niger strain NN059183. Transformants were selected from the Cove-N (tf). Randomly selected transformants were inoculated onto Cove-N plates with 2.5 μM FdU, an agent which kills cells expressing the herpes simplex virus (HSV) thymidine kinase gene (TK) harbouring in pHUda1043. Strains which grew well on Cove-N plates with 2.5 μM FdU and Cove-N plates with 10 μg/ml 5-fluorocytosine (5FC) were purified and subjected to Southern blotting analysis to confirm whether the fcy1 gene was disrupted correctly or not.

The following set of primers to make a non-radioactive probe was used to analyze the selected transformants. For the 3′ fcy1 flanking region:

Forward primer:
(SEQ ID NO: 61)
gaaagctagttctggtcgcattgagc
Reverse primer:
(SEQ ID NO: 62)
gaagttgaaggagatgggtctgga

Genomic DNA extracted from the selected transformants was digested by NheI and XhoI and Southern blotting analysis was preformed using the above probe. Strains of interest were identified by the disappearance of a 3.1 kb NheI-XhoI band and the appearance of a 2.0 kb NheI-XhoI band. Among the strains given the right integration events, a strain NN059186 was selected.

### Example 7

### Introduction of FRT Sites and A. Niger fcy1 Gene at the Neutral Amylase II (NA2) Locus in A. Niger NN059186

The pyrG Gene Rescue in NN059186

At first, the introduced pyrG gene at the fcy1 loci in NN059186 was rescued as follows. The strain NN059186 was inoculated once on Cove-N media containing 10 mM uridine and 1 g/L 5-fluoro-orotic acid (5-FOA). Strains in which the pyrG gene has been deleted will grow in the presence of 5-FOA; those that retain the gene will convert 5-FOA to 5-fluoro-UMP, a toxic intermediate. The colonies that grew more quickly were isolated. The isolated strain was named NN059200.

Construction of pHUda1078 for Introduction of FRT Sites and A. Niger fcy1 at the NA2 Loci

The following primers 3na2-F and 3na2-R introducing a XbaI site and a PmeI site, respectively, were designed to isolate a 3′ flanking region of A. niger NA2 gene fused with FRT-F3 site based on the nucleotide sequences information in EMBL:am270278 and DJ052242:

3na2-F:
(SEQ ID NO: 63)
tctagattgaagttcctattccgagttcctattcttcaaatagta
taggaacttcatgtctccatgtttcttgagcggaagtact
3na2-R:
(SEQ ID NO: 64)
gtttaaacgaagactgatattatggcggaa

A PCR reaction with genome DNA of Aspergillus niger strain NN059183 as template was performed using a primer pair of 3na2-F and 3na2-R. The reaction products were isolated on a 1.0% agarose gel and 2.1 kb product band was excised from the gel. The 2.1 kb amplified DNA fragment was digested with XbaI and PmeI, and ligated into the pHUda801 digested with XbaI and PmeI to create pHUda801-3na2.

The following primers 5na2-F and 5na2-R introducing a NotI site and a SpeI site, respectively, were designed to isolate a 5′ flanking region of A. niger NA2 gene fused with FRT-F site based on the nucleotide sequences information in EMBL:am270278 and DJ052242:

5na2-F:
(SEQ ID NO: 65)
gcggccgcaagagtcaaaagatagcagagc
5na2-R:
(SEQ ID NO: 66)
actagtgctagcgaagttcctatacttgaataggaactcggaatagg
aacttcaagatgaattcgcggccggccgcatg

A PCR reaction with genome DNA of Aspergillus niger strain NN059183 as template was performed using a primer pair of 5na2-F and 5na2-R. The reaction products were isolated on a 1.0% agarose gel and 2.0 kb product band was excised from the gel. The 2.0 kb amplified DNA fragment was digested with NotI and SpeI, and ligated into the pHUda801-3na2 digested with NotI and SpeI to create pHUda801-3na2-5na2.

The 4.3 kb DNA fragment containing T.c.GA gene driven by triple tandem NA2 promoter (Pna2) and AMG terminator (Tamg) was recovered from pHUda440-FRT by NheI and XbaI digestion. The recovered 4.3 kb fragment was ligated to NheI and XbaI digested pHUda801-3na2-5na2. The ligation mixture was transformed into E. coli DH5α to create the expression plasmid pHUda801-3na2-5na2-TC.

The 2.1 kb DNA fragment containing A. nidulans pyrG gene was recovered from pHUda794 by Spell and XbaI digestion. The recovered 2.1 kb fragment was ligated to XbaI partially digested pHUda801-3na2-5na2-TC. The ligation mixture was transformed into E. coli DH5α to create the expression plasmid pHUda801-3na2-5na2-TC-pyrG.

The following primers fcy-F and fcy-R introducing a NheI site at both sites were designed to isolate an entire region of A. niger fcy1 gene based on the nucleotide sequences information in EMBL:am269962:

fcy-F:
(SEQ ID NO: 67)
gctagcgcgaggctatcacggaggctgtgg
fcy-R:
(SEQ ID NO: 68)
gctagcttctgtggttcttgccatgatcgt

A PCR reaction with genome DNA of Aspergillus niger strain NN059183 as template was performed using a primer pair of fcy-F and fcy-R. The reaction products were isolated on a 1.0% agarose gel and 1.5 kb product band was excised from the gel. The 1.5 kb amplified DNA fragment was digested with NheI, and ligated into the pHUda801-3na2-5na2-TC-pyrG digested with NheI to create pHUda1078.

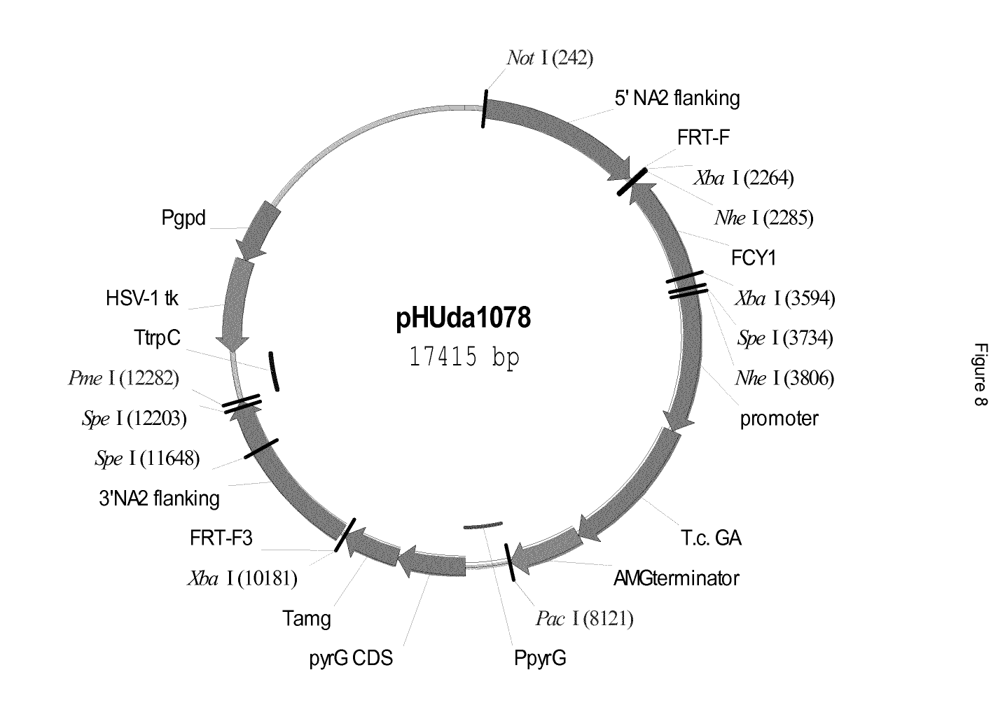

The nucleotide sequence of the A. niger NA2 gene with flanking sequences in pHUda1078 is shown in SEQ ID NO:69; the amino acid sequence of the NA2-encoded polypeptide is shown in SEQ ID NO:70. The nucleotide sequence of A. niger fcy1 in pHUda1078 & 1067 (see below) is shown in SEQ ID NO:71 and the fcy1-encoded amino acid sequence in SEQ ID NO:72. A plasmid map of pHUda1078 is shown in FIG. 8.

Introduction of FRT Sites and A. Niger fcy1 Gene Plus T.c. GA at the NA2 Locus in A. Niger NN059200

The pHUda1078 was introduced into Aspergillus niger strain NN059200. Transformants were selected from the Cove-N (tf). Randomly selected transformants were inoculated onto Cove-N plates with 2.5 μM 5-Fluoro-2-deoxyuridine (FdU). Strains which grew well on Cove-N plates with 2.5 μM FdU and hardly grew on Cove-N plates with 10 μg/ml 5-fluorocytosine (5FC) were purified and subjected to Southern blotting analysis to confirm whether the FRT sites and fcy1/T.c.GA genes were introduced correctly at the NA2 locus or not.

The following set of primers to make a non-radioactive probe was used to analyze the selected transformants. For the T.c.GA coding region:

Forward primer:
(SEQ ID NO: 73)
tcgagtgcggccgacgcgtacgtc
Reverse primer:
(SEQ ID NO: 74)
cagagagtgttggtcacgta

Genomic DNA extracted from the selected transformants was digested by SpeI. By the right gene introduction event, a hybridized signal of the size of 4.4 kb by SpeI digestion was observed probed described above. Among the strains given the right integration events, a strain NN059203 was selected.

### Example 8

### Introduction of FRT Sites and the A. Niger fcy1 Gene as Well as the T.c.GA Gene at the Neutral Amylase I (NA1) and Acid Stable Amylase Locus in A. Niger NN059203

The pyrG Gene Rescue in NN059203

The introduced pyrG gene at the NA2 loci in NN059203 was rescued as follows. The strain NN059203 was inoculated once on Cove-N media containing 10 mM uridine and 1 g/L 5-fluoro-orotic acid (5-FOA). Strains in which the pyrG gene has been deleted will grow in the presence of 5-FOA; those that retain the gene will convert 5-FOA to 5-fluoro-UMP, a toxic intermediate. The colonies that grew more quickly were isolated. The isolates strain was named NN059207.

Construction of pHUda1067 for Introduction of FRT Sites and A. Niger fcy1 at the NA1 and Acid Stable Amylase Loci

The following primers bac-F and bac-R introducing a XbaI site at both sites were designed to isolate a vector sequence of pBluescript II SK-fused with FRT-F and FRT-F3 sites:

bac-F:
(SEQ ID NO: 75)
tctagagaataggaactcggaataggaacttcaagatgaattcgcggccgcg
bac-R:
(SEQ ID NO: 76)
tctagattgaagttcctattccgagttcctattcttcaaatagtataggaacttcagcatgca
agcttggcctccgc

A PCR reaction with pBluescript II SK- as template was performed using a primer pair of bac-F and bac-R. The reaction products were isolated on a 1.0% agarose gel and 2.7 kb product band was excised from the gel. The 2.7 kb amplified DNA fragment was digested with XbaI, and ligated into the pHUda1078 digested with XbaI to create pHUda1078-NA2.

The following primers FLP-F and FLP-R introducing a PacI site at both sites were designed to isolate a FLP expression cassette driven by A. nidulans xylanase promoter (PxInA) and A. oryzae niaD terminator (TniaD):

FLP-F:
(SEQ ID NO: 77)
ttaattaatggaagtgcgttgatcattatt
FLP-R:
(SEQ ID NO: 78)
ttaattaaactagtggagcgaaccaagtga

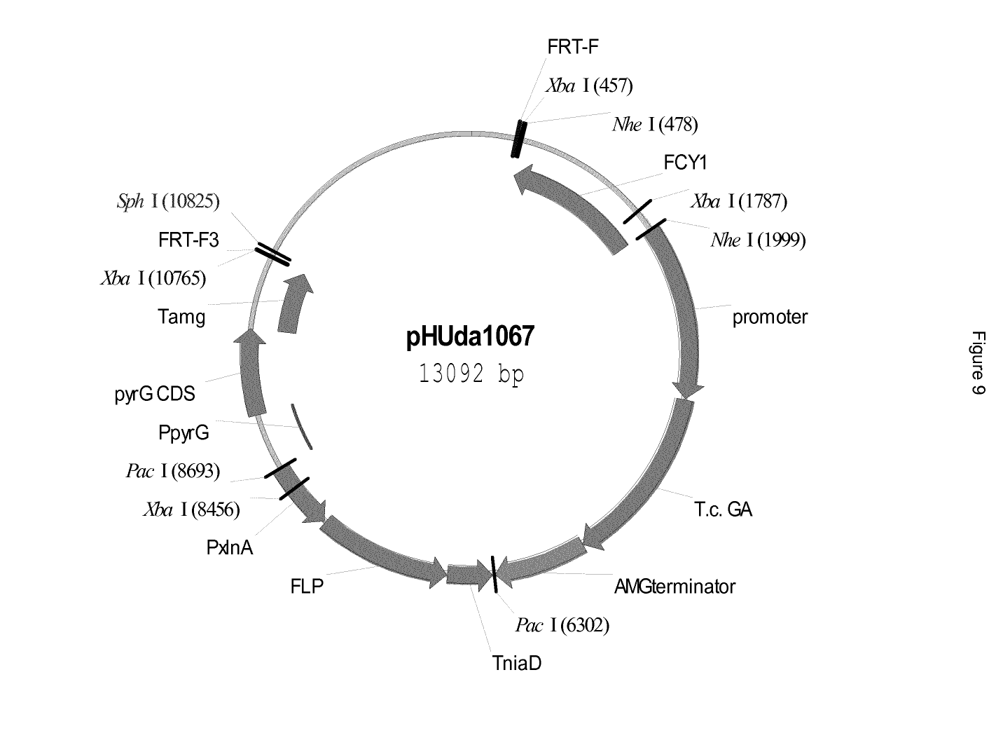

A PCR reaction with pHUda996 as template was performed using a primer pair of FLP-F and FLP-R. The reaction products were isolated on a 1.0% agarose gel and 2.4 kb product band was excised from the gel. The 2.4 kb amplified DNA fragment was digested with PacI, and ligated into the pHUda1078-NA2 digested with PacI to create pHUda1067. A plasmid map is shown in FIG. 9.

Introduction of FRT Sites and A. Niger fcy1 Gene and T.c.GA Gene at the NA1 and Acid Stable Amylase Loci in A. Niger NN059207

The pHUda1067 was introduced into Aspergillus niger strain NN059207. Transformants were selected from the Cove-N (tf) supplemented with 1% D-xylose. Randomly selected transformants were inoculated onto Cove-N plates. Strains which grew well on Cove-N plates were purified and subjected to Southern blotting analysis to confirm whether the FRT sites and fcy1 gene in pHUda1067 was introduced at NA1 and acid stable amylase loci correctly or not.

The following set of primers to make a non-radioactive probe was used to analyze the selected transformants. For the T.c.GA coding region:

Forward primer:
(SEQ ID NO: 79)
tcgagtgcggccgacgcgtacgtc
Reverse primer:
(SEQ ID NO: 80)
cagagagtgttggtcacgta

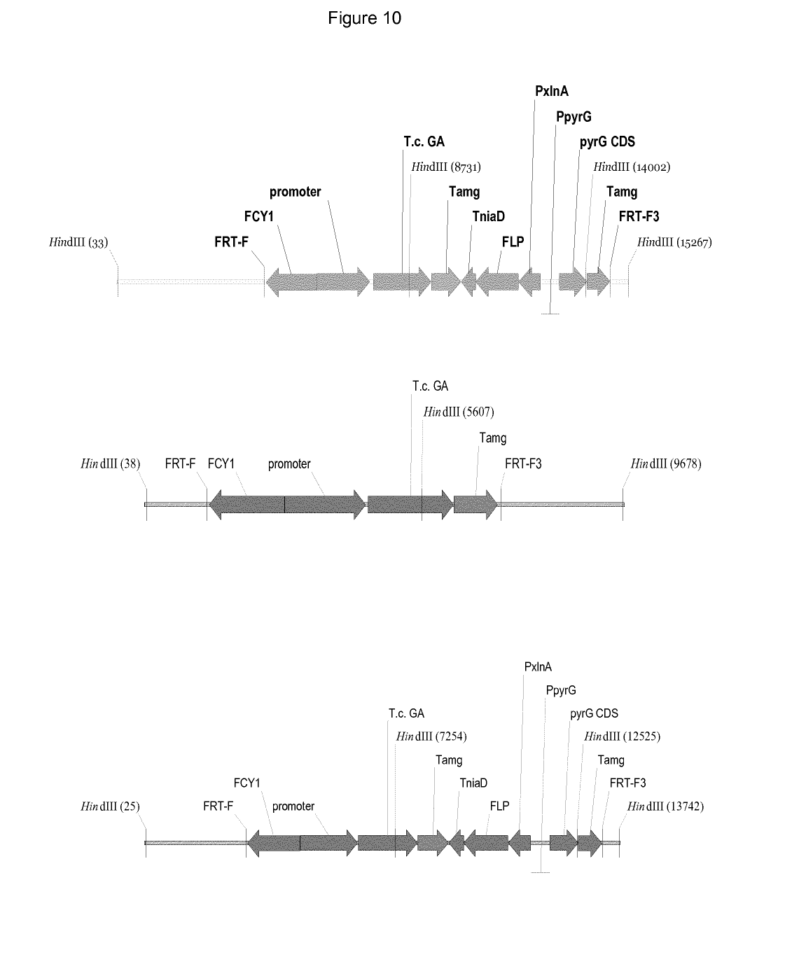

Genomic DNA extracted from the selected transformants was digested by HindIII. By the right gene introduction event, hybridized signals of the size of 8.7 kb (NA1), 7.2 kb (acid stable amylase) and 5.6 kb (NA2) by HindIII digestion was observed when probed as described above. Among the strains with the right 3-copy integration events, a strain denoted NN059208 was selected. FIG. 10 shows the schematic NA1 locus (upper), NA2 locus (middle) and acid stable amylase locus (lower) in NN059208.

NN059203 and NN059208 having 1-copy and 3-copy-T.c.GA genes, respectively, were fermented in shake flasks and their enzyme activities (AGU activities) were measured followed by the materials and methods described above; results are shown in table 1 below. Two-copy T.c. GA strains (1000-7, 18) generated by transformation of either NN059183 or C1997 with pHUda1000 were also fermented.

TABLE 1
The AGU activity of 1-, 2- and 3-copy strains,
wherein NN059203 is normalized to 1.00.

| | | | T.c. GA | AGU |
|---|---|---|---|---|
| Strain | Host | plasmid | copies | relative activity |
| NN059203 | NN059183 | pHUda1078 | 1 | 1.00 |
| 1000-7 | NN059183 | pHUda1000 | 2 | 1.98-2.08 |
| 1000-18 | C1997 | pHUda1000 | 2 | 1.96-2.10 |
| NN059208 | NN059203 | pHUda1067 | 3 | 2.87-3.00 |

### Example 9

### Simultaneous Gene Swapping T.c. GA Gene for JA126 Amylase Gene in the 3 Loci (NA1, NA2 and Acid Stable Amylase) in NN059208 by FLP

The pyrG Gene Rescue in NN059208

At first, the introduced pyrG genes at the NA1 and acid stable amylase loci in NN059208 were rescued as follows. The strain NN059208 was inoculated once on Cove-N media containing 10 mM uridine and 1 g/L 5-fluoro-orotic acid (5-FOA). Strains in which the pyrG gene has been deleted will grow in the presence of 5-FOA; those that retain the gene will convert 5-FOA to 5-fluoro-UMP, a toxic intermediate. The colonies that grew more quickly were isolated. The isolated strain was named NN059209.

Construction of pRika147 for Introduction of JA126 Amylase Gene at Three Loci

The 1.5 kb DNA fragment containing A. niger fcy1 gene was removed from pHUda1067 by NheI digestion. The recovered 1.5 kb fragment was re-ligated. The ligation mixture was transformed into E. coli DH5α to create the expression plasmid pHUda1067-fcy.

The following primers 126-F and 126-R introducing a BamHI site and a PmII site, respectively, were designed to isolate an encoding region of JA126 amylase comprising the secretion signal sequences of A. niger acid stable amylase, catalytic domain of amylase from Rhizomucor pusillus and linker and starch binding domain from glucoamylase of Aspergillus niger:

126-F:
(SEQ ID NO: 81)
ggatccaccatgcggctctccacatcc
126-R:
(SEQ ID NO: 82)
cacgtgtgattacggacacaatccgttatt

The nucleotide sequence of the JA126 amylase gene is shown in SEQ ID NO:83 and the encoded amino acid sequence is shown in SEQ ID NO:84.

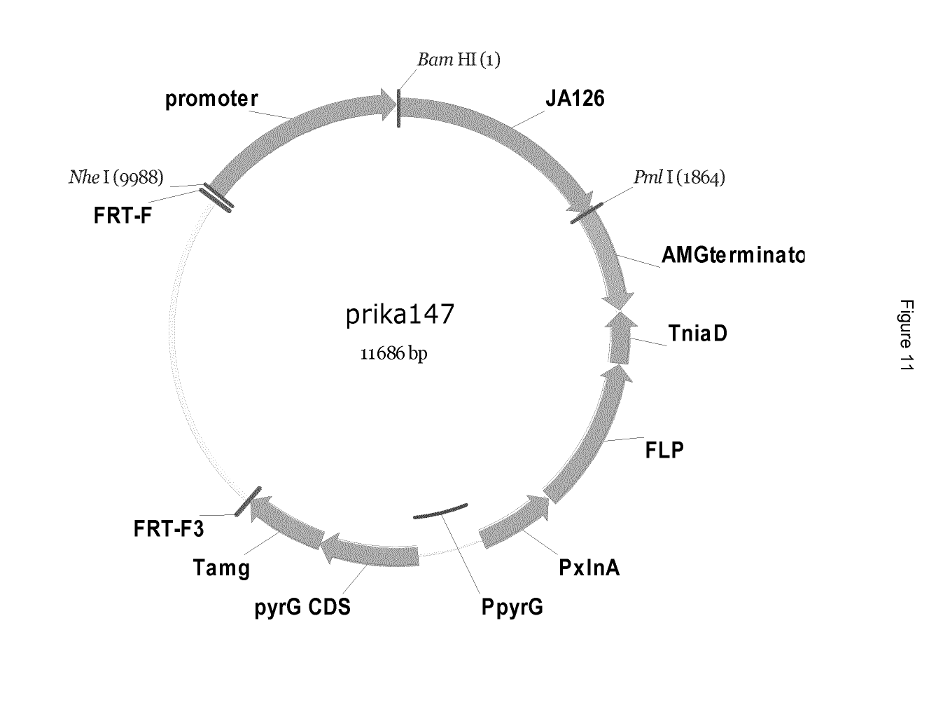

A PCR reaction with pJA126AN as template was performed using a primer pair of 126-F and 126-R. The reaction products were isolated on a 1.0% agarose gel and 1.9 kb product band was excised from the gel. The 1.9 kb amplified DNA fragment was digested with BamHI and PmII, and ligated into the pHUda1067-fcy digested with BamHI and PmII to create pRika147. A plasmid map is shown in FIG. 11.

### Simultaneous Introduction of JA126 Amylase Gene in the 3 Loci (NA1, NA2 and Acid Stable Amylase) in NN059209

The pRika147 was introduced into Aspergillus niger strain NN059209. Transformants were selected from the Cove-N (tf) supplemented with 1% D-xylose and 10 μg/ml 5-fluorocytosine (5FC). Randomly selected transformants were inoculated onto Cove-N plates supplemented with 10 μg/ml 5-fluorocytosine (5FC). Strains which grew well on Cove-N plates supplemented with 10 μg/ml 5-fluorocytosine (5FC) were purified and subjected to Southern blotting analysis to confirm whether the JA126 gene in pRika147 was introduced at NA1, NA2 and acid stable amylase loci correctly or not.

The following set of primers to make a non-radioactive probe was used to analyze the selected transformants. For the JA126 coding region:

Forward primer:
(SEQ ID NO: 85)
tcgaacttcggcgacgagtcgcagttgaa
Reverse primer:
(SEQ ID NO: 86)
cccaacatctcggaaatcctggagaaaccc

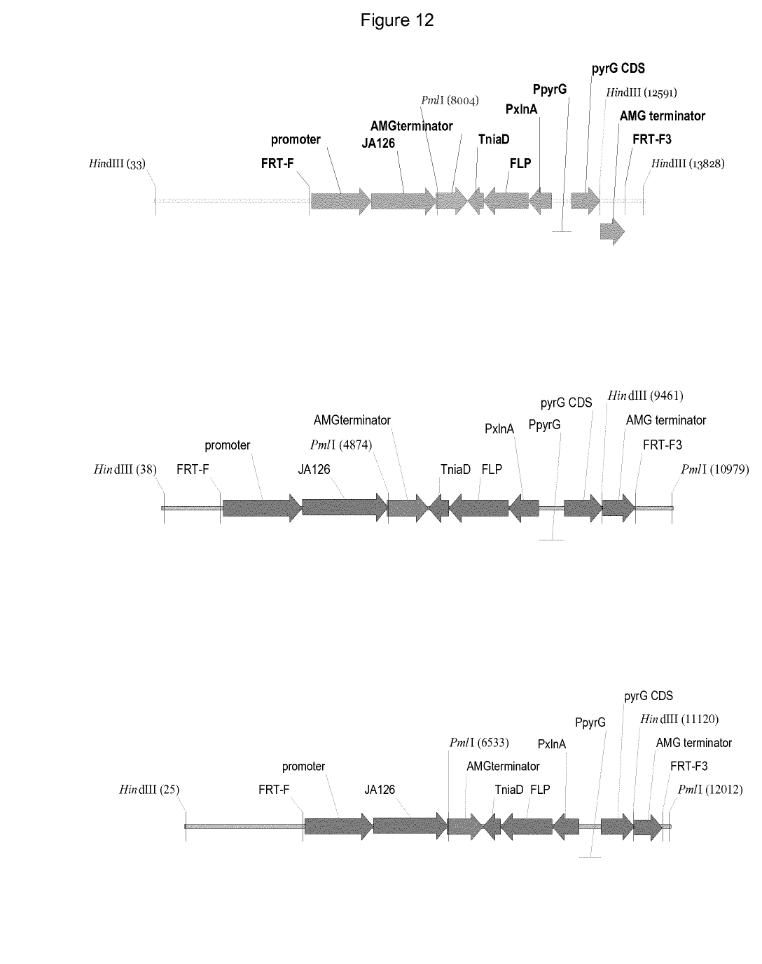

Genomic DNA extracted from the selected transformants was digested by HindIII and PmII. By the right gene introduction event, hybridized signals of the size of 8.0 kb (NA1), 6.5 kb (acid stable amylase) and 4.8 kb (NA2) by HindIII and PmII digestion was observed when probed as described above. FIG. 12 shows the schematic NA1 (upper), NA2 (middle) and acid stable amylase loci (lower) after the correct integration of pRika147 in NN059208.

The frequencies of generations of transformants by Cove-N plates supplemented with 10 μg/ml 5-fluorocytosine (5FC) was approx. 1/10,000 of those by Cove-N plates without 5FC. However, 50% of the generated strains by Cove-N plates supplemented with 10 μg/ml 5-fluorocytosine (5FC) gave right integration at 3 loci, whereas all strains selected randomly by Cove-N plates without 5FC gave right integration mostly at 1 loci, whereas no strains generated without 5FC showed the right integration events. It indicated that the counter-selection using the fcy1 gene worked very well.

Three strains (R147-17, 26, 34) introducing JA126 amylase gene at 3 loci were fermented in shake flasks and their enzyme activities (AFAU activities) were measured followed by the materials and methods described above; results are shown in table 2 below. As a reference, C2325, a single copy JA126 amylase strain generated by ordinary homologous recombination (not shown) was also fermented.

TABLE 2
The AFAU activity of 1- and 3-copy strains,
wherein C2325 is normalized to 1.00.

| | JA126 | |
|---|---|---|
| Strain | copies | AFAU relative activity |
| C2325 | 1 | 1.00 |
| R147-17 | 3 | 2.75-2.96 |
| R147-26 | 3 | 2.82-3.00 |
| R147-34 | 3 | 3.15-3.18 |

### Example 10

### Introduction of FRT Sites and TK Gene at the Amylase B (amyB) Locus in A. Oryzae JaL1196

Construction of a ligD Disruption Plasmid, pJaL1123

Two restriction recognition sites for BamHI and BgIII, respectively, were destroyed in pDV8. First pDV8 was digested with BamHI and then the ends were completely filled in by treatment with Klenow enzyme and the four dNTPs. The resulting 6030 bp fragment was re-ligated providing plasmid pJaL504. Secondly pJaL504 was digested with BgIII and then the ends were completely filled in by treatment with Klenow enzyme and the 4 dNTPs. The resulting 6034 bp fragment was re-ligated providing plasmid pJaL504-delta-BgIII.

By PCR with primers 172450 and 172449 a 2522 bp fragment was amplified containing the HSV-TK gene flank by the A. nidulans gpd promoter and TrpC terminator. The PCR fragment was then cloned into the plasmid pCR®4Blunt TOPO® vector resulting in pJaL574.

Primer 172449:
(SEQ ID NO: 87)
gacgaattccgatgaatgtgtgtcctg
Primer 172450:
(SEQ ID NO: 88)
gacgaattctctagaagatctctcgaggagctcaagcttctgtacagtg
accggtgactc

The A. oryzae pyrG gene from pJaL554 was isolated as 2403 bp StuI-EcoRI fragment, wherein the EcoRI site was completely filled in by treatment with Klenow enzyme and the 4 dNTPs. The fragment was cloned into the unique PmeI site in pJaL574 resulting in plasmid pJaL1022. Plasmid pJaL1022 was digested with SspB1 and the 8574 bp fragment was isolated and re-ligated, resulting in plasmid pJaL1025. Plasmid pJaL1025 was digested with EcoRI and the 8559 bp fragment was isolated and re-ligated, resulting in plasmid pJaL1027. One of two BamHI sites was destroyed by partial digestion with BamHI following treatment with Klenow enzyme and the four dNTPs, whereby the ends were completely filled in. The 8563 bp fragment was re-ligated resulting in plasmid pJaL1029.

From the publicly available A. oryzae RIB40 genome sequence (NITE database (http://www.bio.nite.go.jp/dogan/project/view/AO) primers were designed to PCR amplify the 5′ flanking and the 3′ flanking sequences of the ligD gene (AO090120000322). The primers for the 5′ flanking part, X440700 and X4407007, were tailed with BamHI and EcoRI sites, respectively:

Primer X440700:
(SEQ ID NO: 89)
cagggatccgtctaggctgcaataggc
Primer X4407007:
(SEQ ID NO: 90)
ggagaattcggtcacatc

The primers for the 3′ flanking part, X7164D09 and X7164D10, were tailed with HindIII and SpeI sites, respectively:

Primer X7164D09:
(SEQ ID NO: 91)
gacactagtcgtcggcagcaccggtg
Primer X7164D10:
(SEQ ID NO: 92)
cagaagcttcagagtgaaatagacgcgg

Genomic DNA from ToC1512 was used as template for the PCR reaction. The amplified 5′ and 3′ fragments on 1114 bp and 914 bp were digested with BamHI-EcoRI and HindIII-SpeI, resulting in an 1102 bp fragment and a 902 bp fragment, respectively. The 3′ flanking fragment was cloned into the corresponding sites in pJaL1029 giving pJaL1120. The 5′ flanking fragment was then cloned into the corresponding sites in pJaL1120, resulting in pJaL1123.

Construction of a ligD Minus A. Oryzae Strain, JaL1194.

Plasmid pJaL1123 was linearized with SpeI and used to transform A. oryzae ToC1512 and transformants were selected on minimal medium supplemented 0.6 mM 5-fluoro-2′-deoxyuridine (FdU) as described in WO 0168864. A number of transformants were re-isolated twice and genomic DNA was prepared. The chromosomal DNA from each of the transformants was digested with Asp718 and analyzed by Southern blotting, using the 1102 bp 32P-labelled DNA EcoRI-BamHI fragment from pJaL1123 containing the 5′ flanks of the A. oryzae ligD gene as the probe. Strains of interest were identified by the disappearance of a 3828 bp Asp718 band and the appearance of a 2899 bp Asp718 band. One transformant having the above characteristics was named JaL1194.

Isolation of a pyrG Minus A. Oryzae Strain, JaL1196

The A. oryzae strain JaL1194 was screened for resistance to 5-fluoro-orotic acid (FOA) to identify spontaneous pyrG mutants on minimal plates (Cove D. J. 1966. Biochem. Biophys. Acta. 113:51-56) supplemented with 1.0 M sucrose as carbon source, 10 mM sodium nitrate as nitrogen source, and 0.5 mg/ml FOA. One strain, JaL1196, was identifying as being pyrG minus. JaL1196 is uridine dependent, therefore it can be transformed with the wild type pyrG gene and transformants selected by the ability to grow in the absence of uridine.

Construction of a Aflatrem Gene Cluster (Atm) Deletion Plasmid, pJaL1202

A. oryzae telomere sequences were introduced around the TK expression cassette by PCR with primers T5483H12 and T5483G10 on pJaL574:

Primer T5483H12:
(SEQ ID NO: 93)
gcacatatgatttaaatccctaatgttgaccctaatgttgaccc
taatgttgagcggccgcgtttaaacgaattcgccc
Primer T5483G10:
(SEQ ID NO: 94)
cgtaagcttatttaaatccctaatgttgaccctaatgttgacc
ctaatgttgagaccggtgactctttctg

The amplified fragment of 2595 bp was digested with NdeI and HindIII and the resulting 2582 bp fragment was cloned into the corresponding sites in pU19 giving pJaL835. Plasmid pJaL835 was digested with HindIII, the ends were filled out by treatment with Klenow enzyme and the four dNTPs and then re-ligated to give pJaL955.

Plasmid pJaL554 was digested with Hind and Asp718 and the resulting 1994 bp fragment encoding the A. oryzae pyrG gene was cloned into the corresponding sites in pToC65 giving pJaL1183. A 1535 bp fragment 5′ for the atm was amplified from ToC1512 genomic DNA by primers D5831F08 and D5831F09:

Primer D5831F08:
(SEQ ID NO: 95)
gacgaattcggcgtgggaaattcctgg
Primer D5831F09:
(SEQ ID NO: 96)
ccctacacctggggtacc

The amplified fragment was digested with EcoRI and Asp718 and the resulting 1514 bp fragment was cloned into the corresponding sites in pJaL1183 giving pJaL1194. The 3529 bp EcoRI-NotI fragment from pJaL1194 containing the atm 5′ flank and the pyrG gene was ligated together with the 3529 bp fragment from pJaL955 containing the TK gene, giving pJaL1202. Plasmid pJaL1202 is a plasmid for deletion of the chromosomal atm gene cluster.

### Construction of a Atm Minus A. Oryzae Strain, JaL1268.

Plasmid pJaL1202 was linearized with SpeI and used to transform A. oryzae JaL1196. Transformants were selected on minimal medium supplemented 0.6 mM 5-fluoro-2′-deoxyuridine (FdU) as described in WO 0168864. A number of transformants were re-isolated twice and genomic DNA was prepared. The chromosomal DNA from each of the transformants was digested with SacI and analyzed by Southern blotting, using the 1514 bp 32P-labelled DNA EcoRI-Asp718 fragment from pJaL1194 containing the 5′ flanks of the A. oryzae atm gene cluster as the probe. Strains of interest were identified by the disappearance of a 3230 bp SacI band and the appearance of a 4436 bp SacI band. One transformant having the above characteristics was named JaL1268.

Isolation of a pyrG Minus A. Oryzae Strain, JaL1338

The A. oryzae strain JaL1268 was screened for resistance to 5-fluoro-orotic acid (FOA) to identify spontaneous pyrG mutants on minimal plates (Cove D. J. 1966. Biochem. Biophys. Acta. 113:51-56) supplemented with 1.0 M sucrose as carbon source, 10 mM sodium nitrate as nitrogen source, and 0.5 mg/ml FOA. One strain, JaL1338, was identifying as being pyrG minus. JaL1338 is uridine dependent, therefore it can be transformed with the wild type pyrG gene and transformants selected by the ability to grow in the absence of uridine.

Construction of a Plasmid Containing the TK Gene Flanked by FRT Sites for Integration at the Amylase B Locus, pJaL1258

From the publicly available A. oryzae RIB40 genome sequence (NITE database (http://www.bio.nite.go.jp/dogan/project/view/AO) primers were designed to amplify the 5′ flanking and the 3′ flanks sequences of the amylase B (amyB) gene (A0090023000944). The primers for the 5′ flanking part, D5775F04 and D5775D07, were tailed with NotI and HindIII sites, respectively:

Primer D5775F04:
(SEQ ID NO: 97)
gacgcggccgcgctttgctaaaactttgg
Primer D5775D07:
(SEQ ID NO: 98)
gacaagcttatgctcgatggaaacgtgcac

The primers for the 3′ flanking part, D5775D08 and D5775F05, were tailed with HindIII and NotI sites, respectively:

Primer D5775D08:
(SEQ ID NO: 99)
gacaagcttacagtagttggactactttac
Primer D5775F05:
(SEQ ID NO: 100)
gacgcggccgcgacgagcaactgacggc

Genomic DNA from ToC1512 was used as template for the PCR reaction. The amplified 5′ and 3′ fragments on 1307 bp and 511 bp were digested with NotI and HindIII, resulting in a 1294 bp fragment and a 498 bp fragment, respectively. The 5′ and 3′ flanking fragments were then cloned into the NotI sites in pToC65, resulting in pJaL1196.

The yeast 2μ plasmid FRT sites F and F3 (Schlake T. and Bode J. Use of mutated FLP recognition target (FRT) sites for the exchange of expression cassettes at defined chromosomal loci. Biochemistry 33: 12746-12751) were cloned into pUC19 by annealing of primers F3-1 and F3-2 to form an adaptor having overhang for cloning into the restriction sites BamHI and PstI of pUC19 giving pJaL952:

Primer F3-1:
(SEQ ID NO: 101)
gatccttgaagttcctattccgagttcctattcttcaaatagtataggaacttcactgca
Primer F3-2:
(SEQ ID NO: 102)
tgaagttcctatactatttgaagaataggaactcggaataggaacttcaa

The insertion of the FRT F3 site into pUC19 was verified by sequencing. Then the primers F-1 and F-2 were annealed together to form an adaptor having overhang for cloning into the restriction site Asp718 of pJaL952:

Primer F-1
(SEQ ID NO: 103)
gtaccttgaagttcctattccgagttcctattctctagaaag
tataggaacttca
Primer F-2
(SEQ ID NO: 104)
gtactgaagttcctatactttctagagaataggaagtcgga
ataggaacttcaa

The insertion of the FRT F site in the same orientation as F3 into pJaL952 was verified by sequencing and a correct clone was name pJaL953.

The FRT F-F3 sites were inserted between the amyB flanks by taking a 142 bp Sad-HindIII fragment from pJaL963 containing the FRT sites F and F3 and cloning that into pJaL1196 digested with SacI-HindIII, resulting in pJaL1249 which contains the 5′ amyB flank followed by the FRT F-F3 sites and the 3′ amyB flank.

The pyrG and TK genes were then inserted between the FRT F and FRT F3 sites as follows. A 4838 bp HindIII-SspBI fragment of pJaL1029, where the ends were filled in by treatment with Klenow enzyme and the four dNTP's, was cloned into the SmaI site of pJaL1249, providing a plasmid with the following arrangement of different elements: 5′ amyB flank-FRT F-pyrG-TK-FTRT F3-3′ amyB flank, which was named pJaL1258.

Construction of a A. Oryzae Strain Having the FRT, pyrG, and TK Integrated at the amyB Locus, JaL1386.

Plasmid pJaL1258 was linearized with NotI and used to transform A. oryzae JaL1338; transformants were selected on minimal medium. A number of transformants were re-isolated twice and genomic DNA was prepared. The chromosomal DNA from each of the transformants was digested with XhoI and analyzed by Southern blotting, using the 1294 bp 32P-labelled DNA NotI-HindIII fragment from pJaL1196 containing the 5′ flanks of the A. oryzae amyB gene as probe.

Strains of interest were identified by the disappearance of a 4164 bp XhoI band and the appearance of an 8971 bp XhoI band. One transformant having the above characteristics was named JaL1386.

Isolation of a pyrG Minus A. Oryzae Strain, JaL1394

The A. oryzae strain JaL1386 was screened for resistance to 5-fluoro-orotic acid (FOA) to identify spontaneous pyrG mutants on minimal plates (Cove D. J. 1966. Biochem. Biophys. Acta. 113:51-56) supplemented with 1.0 M sucrose as carbon source, 10 mM sodium nitrate as nitrogen source, and 0.5 mg/ml FOA. One strain, JaL1394, was identifying as being pyrG minus. JaL1394 is uridine dependent, therefore it can be transformed with the wild type pyrG gene and transformants selected by the ability to grow in the absence of uridine.

### Example 11

### Site Specific-Integration by FLP into the amyB Locus in JaL1394

Construction of a the Talaromyce Emersonii AMG Expression Cassette pRIKA99

A Talaromyces emersonii AMG gene containing introns was optimized to provide a synthetic gene (SEQ ID NO:105) for expression in Aspergillus. For cloning purposes, BamHI and XhoI restriction sites were added to the 5′ end and 3′ end, respectively. The synthesized gene was obtained based on the sequence of plasmid pJ241:13509-Huda2. The 2085 bp BamHI-XhoI fragment encoding the Talaromyce emersonii AMG gene and the 9510 bp BamHI-XhoI fragment were isolated from plasmid pJ241:13509-Huda2 and pHUda1000, respectively. The two fragments were ligated together to created pRIKA99.

Site Specific-Integration of pRIKA99 in JaL1394 by FLP

The pRIKA99 was introduced into Aspergillus oryzae strain JaL1394. Transformants were selected on KCl-plates supplemented with 1% D-xylose and 0.6 mM 5-fluoro-2′-deoxyuridine (FdU). Four transformants were re-isolated twice and genomic DNA was prepared. The chromosomal DNA from each of the four transformants was digested with BgIII-DraIII and BgIII-KspI and analyzed by Southern blotting, first by using a 2095 bp 32P-labelled DNA BamHI-XhoI fragment from pRIKA99 containing the AMG gene and secondly after stripping of the filter by using a 731 bp 32P-labelled DNA AfeI-PacI fragment from pRIKA99 containing the A. nidulans xInA promoter as the probes.

The right integration event was identified by giving with: 1) the AMG probe: 7145 bp and 3739 bp bands in the BgIII-DraIII digestion and a 6845 bp band in the BgIII-KspI digestion; 2) the A. nidulans xInA promoter probe a 6845 bp band in the BgIII-DraIII digestion and a 4039 bp band in the BgIII-KspI digestion.

### Example 12

### Aspergillus Oryzae Growth Inhibition by 5-Fluorocytosine (5FC) and Disruption of the Cytosine Aminase

To test that A. oryzae is growth inhibited by 5-fluorocytosine (5FC), spores of BECh2 were streaked on Cove-N(tf) supplemented with different concentration of 5FC (2.5, 1.5 and 0.625 μg/ml). No growth was detected at the lowest 5FC concentration (0.625 μg/ml) indicating that A. oryzae also has a cytosine deaminase. In A. oryzae there is only one orthologous gene (AO090003000802 of the public genome sequence) to the A. niger fcy1 gene (EMBL:am269962), therefore this has been disrupted to verify that this gene is the cytosine deaminase that causes cell death when growing on 5FC.

The AO090003000802 was disrupted by using the bipartite gene-targeting substrate as described in Nielsen et al (2005)Efficient PCR-based gene targeting with a recyclable marker for Aspergillus nidulans, Fungal Gent Biol 43:54-64. Generation of a fragment on 2145 bp containing the 5′ flank of the A. oryzae AO090003000802 gene and a partial pyrG gene (promoter and 2/3 of the encoding region of the pyrG gene) was amplified by PCR. First, a 1036 bp fragment containing the 5′ flank of AO090003000802 was amplified by PCR with primers oJaL132: cagatactggttccttacgg (SEQ ID NO:106) and oJaL133: cgtccacgcggggattatgcgtagaatgcagagatagctg (SEQ ID NO:107) with BECh2 genomic DNA as template. Then second, a 1129 bp fragment containing the 5′ part of the pyrG was amplified by PCR with primers X1111C07: gcataatccccgcgtggacg (SEQ ID NO:108) and oJaL114: ccaacagccgactcaggag (SEQ ID NO:109) with pJaL554 as template DNA. The amplified products were isolated on a 1.0% agarose gel and mixed together and PCR was done with primers oJaL132 and oJaL114 resulting in an amplification product on 2145 bp, which was purified on a 1.0 agarose gel.

Generation of a fragment on 2436 bp containing the 3′ flank of the A. oryzae AO090003000802 gene and a partial pyrG gene (2/3 of the encoding region of the pyrG gene and the terminator) was amplified by PCR. First, a 1011 bp fragment containing the 5′ flank of AO090003000802 was amplified by PCR with primers oJaL 134: cgataagctccttgacggggttgagcactgcttttggatc (SEQ ID NO:110) and oJaL135: gctcacccggcataagttgc (SEQ ID NO:111) with BECh2 genomic DNA as template. Then second, a 1445 bp fragment containing the 5′ part of the pyrG was amplified by PCR with primers X1111C08: ccccgtcaaggagcttatcg (SEQ ID NO:112) and oJaL113: gagctgctggatttggctg (SEQ ID NO:113) with pJaL554 as template DNA. The amplified products were isolated on a 1.0% agarose gel and mixed together and PCR was done with primers oJaL1135 and oJaL135 resulting in an amplification product on 2436 bp, which was purified on a 1.0 agarose gel.

For disruption of the AO090003000802 gene the above two amplified fragments on 2145 bp and 2436 bp was mixed, transformed into A. oryzae JaL1398 strain and transformants was selected from the COVE-N plates. Southern blot analysis was used for verification of the disruption of the AO090003000802 gene. Genomic DNA extracted from 20 transformants was digested with PvuI-SpeI and Southern blotting analysis was performed using the above amplified PCR 1036 bp fragment was 32P-labeled and used as probe. Strains of interest were identified by the disappearance of a 5.5 kb PvuI-SpeI band and the appearance of a 6.9 kb PvuI-SpeI band. At the same time strains were tested for growth on COVE-N plates containing 0.625 μg/ml 5FC and only strains having the expected band on 6.9 kb show growth, which shows that the AO090003000802 gene is a cytosine deaminase. Among these strains one was selected and named JaL1500.

### Example 13

### Introduction of FRT Sites and TK Genes at the Loci amyB and #13 in A. Oryzae

### Construction of A. Oryzae Strain JaL1398

Isolation of a niaD Minus A. Oryzae Strain, JaL828

First the A. oryzae strain 5-58 (WO20099106488) was screened for resistance to chlorate to identify spontaneous niaD mutants on minimal plates (Cove D. J. 1966. Biochem. Biophys. Acta. 113:51-56) supplemented with 1.0 M sucrose as carbon source, 10 mM Na-glutamate as nitrogen source, and 5% Chlorate. One strain, JaL828, was identifying as being niaD minus. Second, the A. oryzae strain JaL828 was screened for resistance to 5-fluoro-orotic acid (FOA) to identify spontaneous pyrG mutants on minimal plates (Cove D. J. 1966. Biochem. Biophys. Acta. 113:51-56) supplemented with 1.0 M sucrose as carbon source, 10 mM sodium nitrate as nitrogen source, and 0.5 mg/ml FOA. One strain, COIs454, was identifying as being pyrG minus. COIs454 is uridine dependent, therefore it can be transformed with the wild type pyrG gene and transformants selected by the ability to grow in the absence of uridine. Third the A. oryzae COIs454 strain was made ligD minus as described in example 10 resulting in A. oryzae strain JaL1390. Fourth the A. oryzae strain JaL1390 was made pyrG minus as described above resulting in strain JaL1398.

Construction of A. Oryzae Strain JaL1523 Having the FRT::TK Integrated at the Loci amyB and #13

For integration of the TK flanked by FRT sites plasmid pJaL1258 was linearized with NotI and used to transform A. oryzae JaL1398; transformants were selected on minimal medium. A number of transformants were re-isolated twice and genomic DNA was prepared. The chromosomal DNA from each of the transformants was digested with XhoI and analyzed by Southern blotting, using the 1294 bp 32P-labelled DNA NotI-Hind fragment from pJaL1196 containing the 5′ flanks of the A. oryzae amyB gene as probe. Strains of interest were identified by the disappearance of a 4164 bp XhoI band and the appearance of an 8971 bp XhoI band. One transformant having the above characteristics was named JaL1450.

Isolation of a pyrG Minus A. Oryzae Strain, JaL1467

The A. oryzae strain JaL1450 was screened for resistance to 5-fluoro-orotic acid (FOA) to identify spontaneous pyrG mutants on minimal plates (Cove D. J. 1966. Biochem. Biophys. Acta. 113:51-56) supplemented with 1.0 M sucrose as carbon source, 10 mM sodium nitrate as nitrogen source, and 0.5 mg/ml FOA. One strain, JaL1467, was identifying as being pyrG minus. JaL1467 is uridine dependent, therefore it can be transformed with the wild type pyrG gene and transformants selected by the ability to grow in the absence of uridine.

Construction of a Plasmid Containing the TK Gene Flank by FRT Site for Integration at the #13 Locus, pJaL1313

In plasmid pJaL835 (US2010062491) the single HindIII was destroyed by opening of the plasmid with HindIII and then the ends was fill out by treatment with 4dNTP's and Klenow following re-ligation resulting in plasmid pJaL955.

Out from the A. oryzae RIB40 genome sequence (www.bio.nite.go.jp/dogan/project/view/AO) primers were designed to amplify the 5′ flanking and the 3′ flanking sequences of the locus #13. The primers for the 5′ flanking part, K6763E12: gacgcggccgccgcgtggaggtctaggac (SEQ ID NO:114) and K6763F01: gacaagcttacaaacccgtgacactcc (SEQ ID NO:115) were tailed with NotI and HindIII sites, respectively. The primers for the 3′ flanking part K6763F02: gacaagcttacgcatgtatgtatgtgtc (SEQ ID NO:116) and K6763F03: gacgtttaaacggatgggtttgccatac (SEQ ID NO:117) were tailed with Hind and PmeI sites, respectively. Genomic DNA from ToC1512 was used as template for the PCR reaction. The amplified 5′ and 3′ fragments on 1065 bp and 1032 bp were digested with NotI-HindIII and HindIII-PmeI, respectively, resulting in a 1052 bp fragment and a 1021 bp fragment, respectively. The 5′ and 3′ flanking fragments were then clone into the NotI-PmeI sites in pJaL955, resulting in pJaL968. The plasmid pJaL968 was digested with NheI-PmeI and ends were completely filled out by treatment with dNTP's and Klenow. The 4548 bp fragment was purified and self-ligated resulting in plasmid pJaL1285.

The yeast 2μ plasmid FRT sites F and F3 (Schlake T. and Bode J. Use of mutated FLP recognition target (FRT) sites for the exchange of expression cassettes at defined chromosomal loci. Biochemistry 33: 12746-12751) were clone into pUC19 by first annealing of primers F3-1 (SEQ ID NO: 15) and F3-2 (SEQ ID NO: 16) to form an adaptor having overhang for cloning into the restriction sites BamHI and PstI of pUC19 giving pJaL952. The insertion of the FRT F3 site into pUC19 was verified by sequencing. Second the primers F-1 and F-2 was annealed together to form an adaptor having overhang for cloning into the restriction site Asp718 of pJaL952. The insertion of the FRT F site in the right orientation same as F3 into pJaL952 was verified by sequencing and a right clone was name pJaL953. Plasmid pJaL953 was digested with SacI-ScaI and the resulting 1866 bp fragment was ligated to an 920 bp ScaI-SacI fragment from pIC19H, resulting in plasmid pJaL1289.

For insertion of the HSV-TK gene between the FRT sites the 4839 bp HindIII-BsrGI, where the ends are completely fill-out bu treatment with dNTP's and Klenow, where cloned into pJaL1289 digested with SmaI. A plasmid having the different elements in the following way: FRT F_pyrG_HSV-TK_FRT F3 was named pJaL1293.

The 4984 bp HindIII fragment harboring the FRT F_pyrG_HSV-TK_FRT F3 part of pJaL1293 was ligated to the 4548 bp HindIII fragment from pJaL1285. A plasmid having the different elements in the following way: 5′ #13 flank_FRT F_pyrG_HSV-TK_FRT F3_{—}3′ #13 flank was named pJaL1313.

Construction of an A. Oryzae Strain Having the FRT, pyrG, and TK Integrated at the #13 Locus, JaL1523.

Plasmid pJaL1313 was linearized with NotI and used to transform A. oryzae JaL1467 and transformants were selected on minimal medium. A number of transformants were re-isolated twice and genomic DNA was prepared. The chromosomal DNA from each of the transformants was digested with NheI-NdeI and analyzed by Southern blotting, using the 893 bp 32P-labelled DNA NcoI-HindIII fragment from pJaL1313 containing the 3′ flanks of the A. oryzae #13 locus as the probe. Strains of interest were identified by the disappearance of a 3896 kb NheI-NdeI band and the appearance of an 5607 kb NheI-NdeI band. One transformant having the above characteristics was named JaL1523.

Isolation of a pyrG Minus A. Oryzae Strain, JaL1540

The A. oryzae strain JaL1523 was screened for resistance to 5-fluoro-orotic acid (FOA) to identify spontaneous pyrG mutants on minimal plates (Cove D. J. 1966. Biochem. Biophys. Acta. 113:51-56) supplemented with 1.0 M sucrose as carbon source, 10 mM sodium nitrate as 4-nitrogen source, and 0.5 mg/ml FOA. One strain, JaL1540, was identifying as being pyrG minus. JaL1540 is uridine dependent, therefore it can be transformed with the wild type pyrG gene and transformants selected by the ability to grow in the absence of uridine.

1. An isolated polynucleotide encoding a polypeptide having cytosine deaminase activity, said polypeptide selected from the group consisting of:
(a) a polypeptide having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO:60;
(b) a polypeptide encoded by a polynucleotide that hybridizes under medium stringency conditions, medium-high stringency conditions, high stringency conditions, or very high stringency conditions with (i) the polypeptide coding sequence of SEQ ID NO:59, (ii) the cDNA sequence thereof, or (iii) the full-length complement of (i) or (ii);
(c) a polypeptide encoded by a polynucleotide having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide coding sequence of SEQ ID NO:59 or the cDNA sequence thereof;
(d) a variant of the polypeptide of SEQ ID NO:60 comprising a substitution, deletion, and/or insertion at one or more positions; and
(e) a fragment of the polypeptide of (a), (b), (c) or (d) that has cytosine deaminase activity.

2. The polynucleotide of claim 1, which encodes a polypeptide having an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO:60.

3. The polynucleotide of claim 1, which hybridizes under medium stringency conditions, medium-high stringency conditions, high stringency conditions, or very high stringency conditions with (i) the polypeptide coding sequence of SEQ ID NO:59, (ii) the cDNA sequence thereof, or (iii) the full-length complement of (i) or (ii).

4. The polynucleotide of claim 1, which has at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO:59 or the cDNA sequence thereof.

5. The polynucleotide of claim 1, which encodes a fragment of a polypeptide having the amino acid sequence of SEQ ID NO:60, wherein the fragment has cytosine deaminase activity.

6. The polynucleotide of claim 1, which encodes a cytosine deaminase polypeptide having the amino acid sequence of SEQ ID NO:60.

7. A nucleic acid construct or expression vector comprising the polynucleotide of claim 1 operably linked to one or more control sequences that directs the production of the cytosine deaminase polypeptide in a suitable expression host cell.

8. A method of using the polynucleotide of claim 1 as a negative selection marker, comprising the steps of:
(a) providing a host cell comprising one or more cytosine deaminase-encoding polynucleotide of any of claims 1-6;
(b) transforming the host cell with an integrative nucleic acid construct which, when site-specifically integrated in the host genome, inactivates at least one cytosine deaminase-encoding polynucleotide, so the resulting host cell produces less or no cytosine deaminase compared with the host cell of step (a);
(c) cultivating the transformed host cell in a selective medium comprising a sufficient amount 5-fluorocytosin, which is converted to an inhibitory concentration of toxic 5-fluorouracil by cytosine deaminase; and
(d) selecting a resulting host cell with reduced or no measurable cytosine deaminase activity which can grow in the selective medium.

9. A method of using the polynucleotide of claim 1 as a positive selection marker, comprising the steps of:
(a) providing a host cell without measurable cytosine deaminase activity;
(b) transforming the host cell with a nucleic acid construct comprising at least one expressible cytosine deaminase-encoding polynucleotide of any of claims 1-6,
(c) cultivating the transformed host cell in a medium comprising a de novo pyrimidine synthesis inhibitor under conditions conducive for the expression of the cytosine deaminase; and
(d) selecting a growing host cell, which comprises at least one cytosine deaminase-encoding polynucleotide.

10. The method of claim 8, wherein the nucleic acid construct further comprises a polynucleotide encoding a protein of interest.

11. The method of claim 10, wherein the protein of interest is an enzyme, preferably a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

12. A method of producing a mutant of a parent host cell, comprising inactivating a polynucleotide of claim 1, which results in the mutant producing less of the encoded cytosine deaminase polypeptide than the parent cell.

13. The method of claim 8, wherein the host cell is a fungal host cell; preferably a filamentous fungal host cell; more preferably an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell or most preferably an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

14. A recombinant host cell comprising at least one chromosomally integrated polynucleotide according to claim 1 operably linked to one or more control sequences that direct the production of the encoded cytosine deaminase.

15. The recombinant host cell of claim 14, which is a fungal host cell; preferably a filamentous fungal host cell; more preferably an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell or most preferably an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

16. (canceled)
